SEQUENCE LISTING (179 sequences)

SEQ ID NO: 1 - DNA, 229 bp, Homo sapiens
tgagctcatttcctggggcgcgcgcgccgggctatttcagcctggcgctgtgcaaacaggacaatttactgcggccaaaagggacccaaattacaatcgtatcacagacaaatatccgccacgccaggtctccaggggccaggaggggcctctctcccggcgcggggggcgggcgcggggtcaggcaggtccgcggggctcggctcggcctcgccgtgccctgatcggc

SEQ ID NO: 2 - DNA, 483 bp, Homo sapiens
cctgggcgtcttgtcccaagtacagagacctggatcctttcccactcatgtgcaacagcccaaaattaaaaacaaaagccatattaaaaaacaaaaccaactttctgccttaaaatattgtgagccagggggcaattagcaattatgctgtattttattatgagaagatagaattctaattggactgatttgaattccacacacctccacagattgttttgggaattaaggtatcagttgtatcggtaattatggtttaccattcaattacccccccacagaaaactgttaaattgtctgtgacggggcttaaatttagctcagacctatgtcctatgaagactgcgcgagtcaatacaagccatccggaaaccaccgggtgccctgtgccaggcggtaattaggggttgaggtttccaaagttttacctgagacagcagggacaagtgcctgggctgggcgtgctcacgtgggggggcttgg

SEQ ID NO: 3 - DNA, 360 bp, Homo sapiens
cttgagcagtagggccaagccctgttcagcctgggaccaagttcccatcaacaaggtggtctgggcagtggccagccagaaagcagtaattactgtcgaggtgcagggaccccaggtagggcccccacctcccacctctgtgtgggcagtgaatgggcctgcccctgggtaaggctgtgtcagcaggcgcctgcccaccccttgctgggttcccaggcccctagagccctctcgtaataggagccatttgcgctgtaaccagtgggtgaccagatttttaatcttggagaccccttggatcccaggcgggaagtgggatttgtcaaatggggagaggcggggctgtctgggaatgccaga

SEQ ID NO: 4 - DNA, 239 bp, Homo sapiens
gtccccttgtttactctcaagcaccccctcccccacccaaggaccaggtctttgtttactgagcatctcagcgatgagctctcaccccctgatttcatcaattataaatgtgctcgctactcaccacacggcaatttgtgacggactgtggtttgtggtgagagtagcaccatccaagttcaccgcagccgcgagtagagatgagggttggggccagacacagggctgtgggggcggca

SEQ ID NO: 5 - DNA, 268 bp, Homo sapiens
ctgaaatggatacgccggttgctccccctcatggctggtctcatttgccttcttcatttttagacacattccaaacttttcagcaaattatagtgtttgccaactggccgtctggggcccaggagagatgctatttatagcgatgctgggatgctgccatcccagagcagcctggtaagaaacggagccagagtgcctgggagtggcgtcctgcaccctggggagaggccagggccctggagcagggtggcaaagctggtggcccgtg

SEQ ID NO: 6 - DNA, 468 bp, Homo sapiens
attccatttcagggcctctcagagtcctgccgtggtgtgcactgtgtgtgtgtttaattttctacatttggatgtgatcctaatccaataaatgcttaggagacttctatagaatagattaatttttactagaaaaaaatataattggctgatgttaaggctactgccctgacaaatctgccttggccatatatctgagaaggtaaaagacccgctacgcttgcacataaatatgccatcttccccacaggccctggagaagcaccccggggaggtttcccttggtgatttattcttcattaataagctctatgctatattaggatcagatttatgactctgcctttctaatatttctgacatttcatctgaaaagaattacaaatgaaatcttgaaactttgccacttctccctgctagtgctctggcactctgtgtccaaggggagatggtgggctggggagaccc

SEQ ID NO: 7 - DNA, 581 bp, Homo sapiens
tttctctgcctttctagcttgggcccagggctcagaaatgtgtggactccctcacagcccctcccagcatccctgccccctcccaactgccttgggcaggtgacacctgtattattgctaagggttaaaaagcccccaaatcaataaaacccattaatgagtgttggtacctcgaaggctacagataaatcccttctactcagtgagttcaatcccataaaacagctctcccctttcaatcctagcattcatttgatagaaaatgtggagaaattttaaaaaggtgacttactaattgcctgtaaaataaaaggcagatggaagctttattacagttgaaggaagtcgggaatattaaggtaaaatgtcaaataacaattgattttccttagacataaaggggcgatttatggcttcctagttactacaaacgagaaattatttgaagttctgaaaagtatgaggagaaataaagattaaatagaagatgaaatcatagggatttctctgggaggtgacttcagtgcccctggggactagaattcatgtggccagtggcctagccagctggggcttggcag

SEQ ID NO: 8 - DNA, 276 bp, Homo sapiens
tggtgaaagacactgcagagaaaagaaagcacagcctgctgccctgggaattaacatgatttaggagacctgcaggtcaccccctcatgactaaaagccatcctggaatgaaggtctgtggctatttctaggcaaaactgtctgataagataaaatagctcaactcctgaccattaagtcgtgaaggccatggccatcgtaaatctcatctttccggccctctggcctgcatgcagtgcagcccagccagtcggtggcagccaccttggtaggaag

SEQ ID NO: 9 - DNA, 259 bp, Homo sapiens
ctgtgaggtttatagtttcatgactgtcagagctttttaaaatgtggtaattttaagtgtgcagcctcccagggtctttcttcttttaattgaagaaataaaccatctcccctaaggcatgcttggcgaaggagaaaggcaggtgcaaggctcacagaggagagcagcagcctagaagggctctgtgtcatggggaagtaaaacatcccagaaacagagagcagaaggccttgactgagccccaggagaggcaggacac

SEQ ID NO: 10 - DNA, 287 bp, Homo sapiens
tggagccagcctgggagactcccagccgcccacttctcggggcctcccttttccagccccttgctttcgaggcagcagtgccattatttggggaaaccagctaaccagataggacagcaaaccggggatttatgtggtgtgggaacagctcaggtttccctccctgtttacccagcagtattttttaaaacagaaatcagcgtgtgggtaaccgcagctgtgagttactagctctggctgtgagggctggggtggggggagtctcttcagagccctctgtccactgg

SEQ ID NO: 11 - DNA, 666 bp, Homo sapiens
gaacaggtgacctggaagatgttccttggtccagtttctgcaaactgccccgcagcatttttagaaaatgttcccttttagattcgatttatcttagccaaattgaccagggaaaataggtgcctacaaatagcgatcactggcaaacaaggagagttattatcttaaattaaggctggtttctaacaacaaaaaaaaacccaccaaatgccactggccaccccccaccaaccccgatccccagcgcatacgtgaggacgatggctccaccaccctccctgtaagtaccaggctcaatgccgggctctgtgcaaagggaaggaaacagacagaggaaggaaggcaagagattagagaagctgacagatgtaaatagcctcagaggagccacactgtcccggcatttctccccagggagccttttgtaccaaggaatctggttgcctgaaagaaaaatgttatcatttatttctttagtcagagctgagctgttttcttcagacatagaaataacctaacatcacacaccaaattgttggccaaatgacagagatacctgtaatgtgggtgtttaataatgtccagggtaaacaatcatggacttggtttcttgggaagggccccgttccccgccacggttgcaaagctacagagggtcttgaatga

SEQ ID NO: 12 - DNA, 320 bp, Homo sapiens
ggctttaaggagcttgtggtcactgagaggattttgcactggaagtacatgcattcaaaatggatacctaagtgtatattttctggtgtaaactatatgtcgactctaactttagcccgggggagctttattatttgtctccctttcatgaaagctataatagaggaagagaaaaccctgcctcgcacattccgattcctaaatacataatttataattttctgggatattatttaagtttattttagttctggatacacaccatccccgtggggtgcttatttaagtatcgggtgggctctggaaaggcctggaatgcc

SEQ ID NO: 13 - DNA, 246 bp, Homo sapiens
tctgggttctggctgataaatggaaatcacctccatgggccacacagtaattaaactcctggcattcttttgacaaaaaaaaagttcctcatgggacattcccacaagctgctgaaggtctgggccctgcaagctcccagctgattaccaaggagttttcgaagttggccttgactgaggatcaaaggaggatgggagttcagggaatgagggtgggggtgggaaatgccttagaattaagttgat

SEQ ID NO: 14 - DNA, 348 bp, Homo sapiens
tttccttctatccctctgtctgtcttactctcagactattaatacaagccctgagtctggctgtacccccagaacatgtgccccgccccctacaacaaaatgctgcccctcccagctaggtctgttgtttgttccttttctgattggcgccaggcttatagaccccatgtaggtagaatataactttccataaataacctctaacccgacctacaatttagccttcaggtttttttccccctcgtggtaatgggattgcagcctgggctgatccatcctgtatcttcaggtcccagaaagcagaccctaggtttggacattgcttggaattcctggtacccccatgtt

SEQ ID NO: 15 - DNA, 600 bp, Homo sapiens
tgggggttctggaaggaatccgtgggaggctgggaggaagatctggcttgtcagcttccctaggaaaaccttcccctgggctggccgcaggctgtaaccggattcctgctccacctctgcatctggcccagggacctcatggcagggaggcccagcgcctggccctttgcccctggacggggtgggccctgggtcatggtggggtgggtggggaggtcaggagggccatggggagggggcgcggtggggtgctttgccctgagaacacaggcctctggcaccccggagcccccggcagctgctggcgtctgtcagccaccttgcggggcgcggccgggggctgctggcccctacatcttcctgacaggcccctcttctgaggccaggaaaaaacaacaacagttcctcccctcacggcaacccatttgttagatgaaggccgggcaccagcacctttaacctcctcaaagtcagcgtttccctgtcaaggccccacagggccagagacagagatggatggaaggagctgtgtgtcgaaaaagccctgtggcctcatgaggagagctctgttttcaggaagggaggggaccccggtttctg

SEQ ID NO: 16 - DNA, 2322 bp, Homo sapiens
agagcctgtgctactggaaggtggcgtgccctcctctggctggtaccatgcagctcccactggccctgtgtctcgtctgcctgctggtacacacagccttccgtgtagtggagggccaggggtggcaggcgttcaagaatgatgccacggaaatcatccccgagctcggagagtaccccgagcctccaccggagctggagaacaacaagaccatgaaccgggcggagaacggagggcggcctccccaccacccctttgagaccaaagacgtgtccgagtacagctgccgcgagctgcacttcacccgctacgtgaccgatgggccgtgccgcagcgccaagccggtcaccgagctggtgtgctccggccagtgcggcccggcgcgcctgctgcccaacgccatcggccgcggcaagtggtggcgacctagtgggcccgacttccgctgcatccccgaccgctaccgcgcgcagcgcgtgcagctgctgtgtcccggtggtgaggcgccgcgcgcgcgcaaggtgcgcctggtggcctcgtgcaagtgcaagcgcctcacccgcttccacaaccagtcggagctcaaggacttcgggaccgaggccgctcggccgcagaagggccggaagccgcggccccgcgcccggagcgccaaagccaaccaggccgagctggagaacgcctactagagcccgcccgcgcccctccccaccggcgggcgccccggccctgaacccgcgccccacatttctgtcctctgcgcgtggtttgattgtttatatttcattgtaaatgcctgcaacccagggcagggggctgagaccttccaggccctgaggaatcccgggcgccggcaaggcccccctcagcccgccagctgaggggtcccacggggcaggggagggaattgagagtcacagacactgagccacgcagccccgcctctggggccgcctacctttgctggtcccacttcagaggaggcagaaatggaagcattttcaccgccctggggttttaagggagcggtgtgggagtgggaaagtccagggactggttaagaaagttggataagattcccccttgcacctcgctgcccatcagaaagcctgaggcgtgcccagagcacaagactgggggcaactgtagatgtggtttctagtcctggctctgccactaacttgctgtgtaaccttgaactacacaattctccttcgggacctcaatttccactttgtaaaatgagggtggaggtgggaataggatctcgaggagactattggcatatgattccaaggactccagtgccttttgaatgggcagaggtgagagagagagagagaaagagagagaatgaatgcagttgcattgattcagtgccaaggtcacttccagaattcagagttgtgatgctctcttctgacagccaaagatgaaaaacaaacagaaaaaaaaaagtaaagagtctatttatggctgacatatttacggctgacaaactcctggaagaagctatgctgcttcccagcctggcttccccggatgtttggctacctccacccctccatctcaaagaaataacatcatccattggggtagaaaaggagagggtccgagggtggtgggagggatagaaatcacatccgccccaacttcccaaagagcagcatccctcccccgacccatagccatgttttaaagtcaccttccgaagagaagtgaaaggttcaaggacactggccttgcaggcccgagggagcagccatcacaaactcacagaccagcacatcccttttgagacaccgccttctgcccaccactcacggacacatttctgcctagaaaacagcttcttactgctcttacatgtgatggcatatcttacactaaaagaatattattgggggaaaaactacaagtgctgtacatatgctgagaaactgcagagcataatactgccacccaaaaatctttttgaaaatcatttccagacaacctcttactttctgtgtagtttttaattgttaaaaaaaaaaagttttaaacagaagcacatgacatatgaaagcctgcaggactggtcgtttttttggcaattcttccacgtgggacttgtccacaagaatgaaagtagtggtttttaaagagttaagttacatatttattttctcacttaagttatttatgcaaaagtttttcttgtagagaatgacaatgttaatattgctttatgaattaacagtctgttcttccagagtccagagacattgttaataaagacaatgaatcatgaccgaaagaaaaaaaaaaaaaaaaaaaaaa

SEQ ID NO: 17 - DNA, 242 bp, Canis lupus
tgagctcatttcctggggcgcgcgcgccgggctatttcagcctggcgctgtgcaaacaggacaatttactgcggccaaaaggcgcccaaattacaatcgtatcacacacaaatatccgccacgccaggcctccggaccggcccctccccgcggggagggtcgcgccccccggcgcgcccgtcaggcgggtccgcggggctcagcggcccggcctcgcccggtcctgctgcgcccctgccggc

SEQ ID NO: 18 - DNA, 448 bp, Opossum
aaaggaaatgctccagagaatgacctcctcctttccccgtggtccagctcttcctcctgctcgtctctgaattttctgtcttaaggaatccaagtcgcagacagcgcagaatagcactttttcattatcataattcaaattacctgtttaagtcggtcaaatgaaaaaatagccgttcagcccccgcctgtggcctggcagagggaaccttgcgaagcgtgtcctgattcggagctgaagcggtccggcccttccctcccgcctgggctcacagacttcaggctccacgtgccatgagctcatttcctgggggggtgtgagtgtgtgtgtatgtgaatgtgagcttctatttcagcctggctccgtgcaaacagggcaatttactgtgaccaaaaggggcccaaattacattcgtaacatagaaaaatattctccttgccaggccacc

SEQ ID NO: 19 - DNA, 126 bp, Mus musculus
gagcctctcgggactcatgccagcgactcccactctctccgagcctttggtggcagacagagcccgaggcacgttttcatgctcataattcaaattgcctgttgaagtcggtcaaatgaaaaaata

SEQ ID NO: 20 - DNA, 165 bp, Rattus sp.
gaggagcctcttggggacccatgccagcaactcccactctctccgaacctttggtggcagacagagcccgaggcacgttttcatgctcataattcaaattgcctgttgaagtcggtcaaatgaaaaaatagccgcgccgggcggtcgcggccgggcgccgggcag

SEQ ID NO: 21 - DNA, 317 bp, Gallus sp.
gcagacagcacggaattggatcatggtcattcaaaccacccgtttaaggcagagacgtgaaaaaaatagccctcagcccacacattgtggcctggcacacagcagcgtccgaagatgaatgcagttgatgagattaatttgttgggcccttacttatcacctgcagacgaggctccgtgcatcatgagctcatttcctggtgctatttcagcctggctccagccaaacagggcaattagctgcggccagcagggctgtatggttaaaatgctctcctggttcagccacacgacccccaggctccagcatggccttct

SEQ ID NO: 22 - DNA, 502 bp, Mus musculus
ccctgggatctcacagaatcccccagtccagagacccagatctttttcctctcaggggatgtgatctcaaatttcaagcaaacacctgaaaataaaagccaaattaaaaaacaaaaccaacctttcctgccttaaaatattgtgagccagggaagcaatctgccttgacgctgcgttttattgtgaggtagaattctaactggactggcttgggttcggtacacctccacacattgtttggggaattgagggatcagtagtattggtaattatggtttagcattcaattacccccacagttgaaaaccgttaaattgtctgtgacagggcttaaatttagcccagtctcatgtcctgtgaagacttcacgagtcaatacaagccatccggaaaccacaaggtccctgtgccaggtggtaatgaccagttgggttgaggttttttatctgggacatcgggacaagggacaagctgacctacagggcacttgtgttgggggtgg

SEQ ID NO: 23 - DNA, 172 bp, Gallus sp.
attacgaacaagtggaattcaaattaggctgattcgagcctcagcaaccccctcagattggattggtaattaagggactcgtactggtaattatggtttaccatgaaattaccctcccttctcccctctccccccaaaaacaccaaattagccacgagtggaattatattta

SEQ ID NO: 24 - DNA, 361 bp, Mus musculus
cctgagctgtctgccaagccctgctcccctgagaccaggttcccatcactggggtggcctgggcagctgccagccaacagcgttaattactacggacttgcagggagctcagcgagcccccaccccctacattcccatttctaagtgggaactgaacgggcccaccatgggtaaaaggggtgtgagcaggcacctgttcatcccttgctgggttcccaggcccctagagctcctggtaatagtgaccggttgccctgtaagcagtgggtgagcaggtttttaatcttggagacccctcagataccagctgggaagtgggatttgtcaaaacgggagggaggtcctgtctgtgaatgtcaga

SEQ ID NO: 25 - DNA, 232 bp, Mus musculus
ggccttctgcatcctatcccctatcctaacccaagggccagggctttgttcattgagacatcttgggatcagttctcaacccctgatttcatcgattataaatgtgcttgtttctcacggcaaggcaatttgtgttgaccgtggtctgtggtgacactaacaccttccgggctcaccacagcctcggcagagaggagggaggagggccagatacagggctgtggaggtggca

SEQ ID NO: 26 - DNA, 293 bp, Mus musculus
ctgaaacagatctgccacttgcaccctgccccaccccgccacccccaccccgagcctggtctcatttgttgtcttcatttttagacacattccaaacttttcagcaaattacaatgtttgccaactggccgtctggggctcaggagagactatttatagagcattgggatgctgccatcctagagaggcctggtaagaaactgggcgagtgctgtgcccagaagtggtatcatgtggcctgggggagaatgaggctccaggagcagggagccaagctggatctacggtccggg

SEQ ID NO: 27 - DNA, 125 bp, Gallus sp.
ctccccatggctgcagcctcctttgtttttatttgacctttatagcgtcgacgcttcaggcccctgtggcaaattataactgtgtttgccagattgtcgtctgggaccgagggaaggagctattt

SEQ ID NO: 28 - DNA, 452 bp, Mus musculus
atcctatcttagccaagtggcgcgggttccctgtgtatgtggagtgtgtatttaatcttctatattttggatgagttcctaatccaataaatgcttaggaaatttctatagaacagattaatttttactagaaaaaaatataattggctgatgttaaggctactgccctgacagatcctgccttggctatctatctgagaaagcgagaggcctcataaatatgccatcttcccctgggccctgagagcactcctcaggggctcctgccgtgatttattcttcattaataagctctatgctgtattaggatcagatttacgactctgcctcactaatatttctgacatttcatctgaaaagaattccaaacgaaatcgtgagacattaccacgtcttccctgctagcgttccaatactgcacacctaaggttgctgctgggatgaggtggccc

SEQ ID NO: 29 - DNA, 619 bp, Mus musculus
tccctcttgttccttcacttgggcccacagagctcagagttgagtggatccctccacctcgcccagcccctgccccctcccaactgccacaggcaggtgacacctgtattattgctaagggttaaaaagcctccaaatcaataaaacccgttaatgagtgttggtacctcgaaggctacagataaatcccctctatcctgagagttcagtcccataaaacagggctcccctctccatcccagcatccatctgacagaaaatgtgaagaaatttttaaaaggtgacttactaattgcctgtaaaataaaaggcagatggaagctttattacagtcgaaggaagtcaggaatattaaggtaaaatgtcaaataacaattgattttccttagacataaagggacgatttatggcttcctagttagtacaaatgagaaattatttgaagttctaaaaagtatgaggagaaataaagattaaatagaagatgaaatcatagagttttttttttttttctgggaggtgacttcagtgtccccagagactagaattcatgtgactgaggggtgggggaggttgtggggagaggtaagttaagacagatggggcttggcttggccag

SEQ ID NO: 30 - DNA, 305 bp, Gallus sp.
aatcaactccacgccacaatgcatctcttttcttcttttttcccctttaattatggagttggtttaacagcaaatgagaagaaatgtttaaaaggtgacttattaattgcctgtaaattaaaaagcagacggatgctttattgcagttgagagaagttagtaatagtaaggtataatgtcaaatggcaattgacttctttagggttctcttttcttttctaacacataaaggagtgatttatcgcttcctgcagactgagggataaatcatcctggaagtctgaaaactggggctaaataaagat

SEQ ID NO: 31 - DNA, 273 bp, Mus musculus
tggtagaagatcccgcatggaaaacacgccatgggcttgtcctgagtgttaacatgatttaggagatacacaggtcagcccctcatgactgagagccatcctggagtgaatctcagtggccatttccaggcaaaagcacccgatgagatgaaatagctcaattgctgaccattaagtcatgaaggccatggccgttgtaaatctccaactcagcacagctttggcctgcaaggagtgctcggcactgccaggaccagccacctctgtgggaag

SEQ ID NO: 32 - DNA, 260 bp, Canis lupus
ccatgaggtttatggtttcatgaatgtcgaagcattttaaaatgtggtaatttcgagtgtgcagcctcccagggcctctctccttttaattgaagaaataaacaatcccccctaaggcatgctcggccacggagaaaggcaggtgcagtgttcaggaggggagcacggcagcctggcaggactctgttgcggagaagtaacatgtccgacgagatggagagctagtcggcttcgcctgagccccgggccagacaggtcgc

SEQ ID NO: 33 - DNA, 251 bp, Rattus sp.
ggagtgggaggcaggagggagcccttaagttcagggcagagaagagtcagggcagctgagaccccacgtggggtttatggtttcatgagtgtcggagtgataaaaatgtggttatttccagcctcccagagtctgtcttgctctaattgaagaaataaacaatctaccctaaggcatggttagcctaggacagaagtgtgtgggagatctgctggggggagaacagcagcctggaaagactttgcgctggg

SEQ ID NO: 34 - DNA, 294 bp, Mus musculus
cagagaacaggtcagagctcccagcctggggagtggggaggcaggagggagccctttagttcagggcagggaagagtcagggcagctgagaccccacgtggggtttatggtttcatgagtgtcagagtgataaaaaatgttgttatttccagcctgtagcctcccagagtctgtcttgttctaattgaagaaataaacaatctaccctaaggcacggttagcctaggacagaagcgtgtgcgatatctgctggggagaacagcagcctaggactttgtgctgagcagattgtcc

SEQ ID NO: 35 - DNA, 270 bp, Mus musculus
tgggagcctgggacaccagccactcccttcggcccccctttccaactctttcattccaagcagcagggccattatgtggaggaaacagctaagcagataggacagcaagcccgcgatttatgtggtgtgggaacggctccgctttccctcactgtttacccagcagtatattttaaaacagaaatcagcatgtggagaaccgcagctgtgaattatgggctctggctgggagggtcatgagggcacctctttcagagtgctacccgcaag

SEQ ID NO: 36 - DNA, 218 bp, Rattus sp.
ccactctcttcaggccccccctctttctttccaggtagcaggaccattctgtgggggaaacagctaagcagataggacagcaagctggcgatttatgtggtgtgggaacggctccggtttccctcactgtttacccagcagtattttttttaaaacagaaatcagcatgcagagaaccgcagctgtgaattatgggctctggctgggagggtcacaag

SEQ ID NO: 37 - DNA, 484 bp, Canis lupus
agtccggggcggcctctgggtgagggaaggaaacaggcagaggaaggaaggccagagattagagaaactgacagatgtaaatagcctcagaggagccacactgtcccggcgctcctccccagggagccttttgtaccaaggaatctggttgcctgaatgaaaaatgatatcatttatttctttagtcagagctgagctgttttcttcagacatagaaataacttaacaccgctacaccaaattgttggccaagtgatagagatatctgtaatttgggcgcttggtaacatcctaggtaaacagccagggacttggtttcttgggaggggccctgttcccggtcaacaatgctacaaagctcagagggacacagatggaagaaaagcttttgaaaaagttgttccagaactctcgcctgctgtcgagctccaaggaagctggatttttcatccctgctgctcaaaccatgctttgccaaccaaac

SEQ ID NO: 38 - DNA, 204 bp, Opossum
accctcctttctctccaacttgtccaggtttgctagcaacatttttagcaaactttcacttgtagatgcaactactcccagccaaattgactagggggaaaaataagtccctacaaatagcaatcactttggaatgaggagagtaattatcttaaattgaggctggtttctgaggggaaaaaaacaccaccaccaacacccagc

SEQ ID NO: 39 - DNA, 674 bp, Mus musculus
gagcatgtaacttagaccgccatccctggtttcgtgccggcacacggtccagccacatttttagaaaaacgttcccttttaaatttgacttgtcttacttagccagattgacagggcgggggtggataagtccctatgaatagtgagcacacgtgaatgaggagactcattatcttaaattgagactggtttctaagaaaaagagaaaggggaaaaagtcactgggccgacagcttccccccttcccccagcccgtagaggaccagggctttgccaccctcctccctgtgagtacacagactccactgggccccagcaagaggaaggaaacagacagagggaggaaatcaagagattagagaagttgacagacggaaatagcgactcaggagccgcgctgtcccggcctcgttccccagggagccttttgtaccacggaatctagttgcctgaatgaaaaatgatatcatttatttcttttgtccaggttgcactgttttctgcagacacagaaataacctaacaccgcaacacccgactgttggccaagtgatagagttatctgtcatcttggccctcaataatgtcctgaataaacaaccacaggcttggtgttctagggaggggccctgctcccccatccccgtgatgactctgcaaagaggcttgaatga

SEQ ID NO: 40 - DNA, 448 bp, Rattus sp.
cccccgcccccatcccatagaggaccagggccctgccaccctcctccctgtgagcactggctccactgggccccagcaagaggaaggaaacagacagagggaggaaatcaagagattagagaagctgacagacggaaatagcttctcaggagccgtgctgtcccagcctggttccccagggagcctttcgtaccacgggatctagttgcctgaatgaaaaatgatatcacttatttcttttgtctgagtggcactgttttctgcagacacagaaataacctaacaccgcaatacccaactgttggccgagcgatagagttatctgtcatcttggccctcaatagtgtcctgagtaaacaaccacaggctgtggtgttctagggtggggccctgctcccccccatccctccacccccgtgatgatgttgcaaaaggggcttgaatgaga

SEQ ID NO: 41 - DNA, 485 bp, Canis lupus
cctctcttcctgagcatttggaccacacagaggcctcccgtgctcttggctttggctttaaggagcttgtggtcaccgagaggatttcacactggaggtacatgcattccaaattgatacctaactgtatattttttggtgtaaactatatgtcggctctaactttagcctgggggagctttgttatttgtttccctttcatgaaagctataatagaggaagtgaaaaccttgccttgtacattccaattcctaaatacacaatttacaattttcggggatattatttaagtttattttgattctggagtctcaccatccccatgggtagcttatttaggttccgggtggactctagaaaggcctggaatgtctcaaagggagggaaggacgatcttcatgttcattcaatagcagaacctaggggctttgcaggatcttggaagcttccccaacaccagctgaggggtctcagtgtgggggctg

SEQ ID NO: 42 - DNA, 301 bp, Opossum
ttaggaccacacagaaggcatactttgcttctggttttggttttaaagggccctgtggtcatcccagaggatatcacactgaaagttcatgcattttaaattgatgtgaaacttcatattttctagtgcaaactatatgttgagtctaattttaacctcagagagctttattattcttttccttttagtgaaagctataatataagtaggaaaaaaaacttgacttgttctctccatttcctgaatatacaattttacaattttctgagatattatttagacttatttttacttttcagcc

SEQ ID NO: 43 - DNA, 313 bp, Mus musculus
ggctgtgagcaccttgtgggtcccagagagatttcccgctggaagtaggtatgctccaaattgatgcctgactggatgctttctggtgtaaactatatgtcgactctaactttagcccaagcgagcgctcttatttgtttcatgaaagctataatacaggacgagacaactcacctcatacattccgatttctaaatacacaatttacaattttctgggatattatttaagtttattttggtgatggagtctcgccatgcccgttgagcgtttatttaagttctgggtggtcactggaaaagcctgggttgcc

SEQ ID NO: 44 - DNA, 292 bp, Rattus sp.
ctggagagatctcccaccggaagtaggcttgctccaaattgatgcctgactggatgccttctgctgtaaactatatgtcgactctaactttagcccgggggagcattcttacttgtttcatgaaagctataatacaggacgagacaactcgcctcatacattccgatttctaaatacacaatttacaattttctgggatattatttaagtttatttttgtgatggagtctcgccatgcccgttgagcgcttatttaagttctgggtggccactggaaaagcctgggatgcct

SEQ ID NO: 45 - DNA, 802 bp, Canis lupus
aaccttagactggatctaccctagagttctggtttgcagctccaatgggaaccaatccccaggacacatttcactctttctctttcccaccacgtacacacaacacagccttttgctgggatttgacagggaaaatcacaatggttgatggttcccctacaagctgctgcaaagccctgtcagaaaaactagattggccactgaacaggaggaaacaacgtggggctgcccaacagcggccaccccacgagattaatgggtaaaactttaatttatccaggcaccttgctttcagagagcctcccgtgccctcttggctgcccctaacccaaacccctcctgttttgctaatgataaatgacaagtagtggacatgattaattaaaccgcccccagtagccagtggtaaaacccatggccacaagcagaggagacattcaaagcattcccactgcccctgcccagggcacaggccccaggctccatcctgctctggctagaacaggccccagtggccacggcctcgcacaaagggccttcctaggaactgtctgggctcaggtctgtcaaatggaaatcaccaatacgggccacacagtaattaaactcctggcattcttttgacaaaaaatgttcctcttgggacattcccacaggctgttgaagctgagtcggggccctataagctctcagctgattaccaagggatttttgaagttagccttgactgtggatcaaaaggggatgggaattcacggggtgctggtggagatgggaagtgccttgtaacagaatttatcat

SEQ ID NO: 46 - DNA, 167 bp, Opossum
tgatgggtaaaattttaatttctttgggcggtttgtatttggagggtctccctgactccggcccctatcccttacccccaccagaccccaacctctgattttgctaatgataaatgacaagatgtggacagttattaattaaactgtccatagcgataaggtctgag

SEQ ID NO: 47 - DNA, 245 bp, Mus musculus
tctgaattctgacaaatgaaaaccacctctccagccatgggatgggataattaaactccctgaaaaaaaaaaaaatcaaagatcgttcctctagggacattcccacgagctgcagaaggactggctgggatctttgagctctcagctaggactgagggattttgaagttggccttgactggaggagattggagggggaggagagttgggatgggtgggtaggaaatgccttgtaattaaatttat

SEQ ID NO: 48 - DNA, 145 bp, Rattus sp.
cctctccaacccctcctggtttgctaatgacaagcaatggacatttattaattaaaccgcccacagaggccagtggtaaaaccgaaggccacatgcagcggaggctttcaaagcattcctactgatcatgcctggggcccagccc

SEQ ID NO: 49 - DNA, 130 bp, Gallus sp.
tggcaggtccccacctcctgtttcggtaatgataaatgacaagatgtcgacagttattaattaaagcacccggtgtagcaacgccaaccagaagggctggggaagcaaaatgtgcagcattccttcctcc

SEQ ID NO: 50 - DNA, 442 bp, Canis lupus
tgtttccctctgcccctctgtctgactcccaagactattaatacaaaccccagtctggctgtgccaccagaacatgtgctccccacagcagaatgccacccctcccagctgagcctgttgtctgttccgtttctgatcggcgccaggctcatagaccccatgtaggtagaatataactttccataaataacccctagcctgacctacaatttaggctttgggtttttttcccctcgtggtaatgggattatagcctgggctgatccctcctgggtcttggggtcccagaaagcagaccctaggtcatagcccctgggcattgcttggaattgccggttcccccatgcctcctggcacctggtgaggacttggaacgtgtcgaggaaactggcggatctctctcgtgcctccacgaacagccgcgcagcttcagccctggggc

SEQ ID NO: 51 - DNA, 478 bp, Mus musculus
tgagaagtgagtggtgtgtgcatgtgtgtgtgtgtgtgtgtgtgtgtgtgtgtgtgtgtgtgtttccttctgtctctctgtctgcttgactctcagcctattaatacaagccctgtgattaatacaagccccgtgtctggctgtaccccaagcacacgtgctccctgccatagagtaccacccctcccagatcagcctgttgtttgttctttctgatgggcgccagccttgtagaccccatggaggtagaatataactttccataaataaactctaacctgacctacaatttggaccttgggtttttttttgttttgttttgtttttttttgtttgtttgtttgttttttgttttttccctctttgtggtaatgggatcacagcccaacctagtctctcctgcatctgtctgtcccagaaagcaagtcctaggtgtgtttgggtttgggcatcacttggactctctgatgcccctcat

SEQ ID NO: 52 - DNA, 440 bp, Rattus sp.
tgagaaacgagtagggtgtgtgtgtgtgtgtgtgtgtgtgtgtgtgtgtgtgagagagagagagagagagagagagagagagagagagagagagagagtttccttctgtctctgtctgactctcagactattaatacaagcctcatgtctggctgcgccccaagcacacgtgctccctgcaatggagtgccacccctcccagctcagtcctttgtttgttctttctgatgggcgccagcctcatagaccccatagaggtagaatataactttccataaataaactctaacctgacctacaatttagaccttgggctcccccccccctttgtggtaatgggatcatagcccaagctagtctctcctgcatctgcccatcccagaaagcaagccctaggtgtgcttgggtttaggcatcacttggactctctgatcccacat

SEQ ID NO: 53 - DNA, 116 bp, Opossum
ccgattagcttcaggctcacaaatcccatatggttagtatatgacattccataaatagcccaaaacacgccctacaatttagcctgtgggttttccccctcatgctaacaggacag

SEQ ID NO: 54 - DNA, 587 bp, Mus musculus
tgagatggaaggagcccacaggaggggtgggctgaagggaagatggggctgtcagctccttaggaaacccttggggcctgacctggcaaactgtaaccagatccctgttgggctgtcctcctcacatctggctcagggtccccattgcagggaggcccccttggccctttgccctgggaaggggtgggcaccgggtcacggtggggtgggtggggaggtcagaaagggcatgaggcgggggcacagcggggtgctatgtgctgggaacataggcctggcaccccggtgccccagcagctgctggcgtctgtcagccccccctaggggcaccacaaggccgccacctctacatcttcctgacaggtccctcttctgaggccaggaaaaaacaacagccccctcccacagcaacccatttgtcagctgaaggccggccctgcacctttaaccttctcaaagtcagcagttccctgtcaaggccctgtggggccaagagacaaagatggatggaggtggctgtttgtagaggaaagctcttggctgcagccaaggggagtggtccggaagggaggggctcctggcttctg

SEQ ID NO: 55 - DNA, 562 bp, Rattus sp.
  misc_feature (189)..(189): n is a, c, g, or t
ggaggggtgggctaaggggaagatggggctgtcagctccttaggaagccctggcagtaggcaagctgtaacaagatccctgttgggccatccttctcacatctggctcagggtccccatcccagggaggccccgtggccctttgccctgggaaggggtgggcactgggtcacggtgggggggggtgggngaggtcanaaagggcatggggcggggcacagcagggtgctatgtgctgggaacacagggcctggcaccccagtgccccagcagctgctggcgtctgtcaacccccctgggggcaccacaaggccaccacctctacatcttcctgacaggcccctcttctgaggccaggaaaaaacaacagccccctcctacagcaacccatttgtcagctgaaggctgtccctgcacctttaacctcctcaaagtcagcagttccctgtcaaggccctgtggggccaggagacaaagatggatggaggtgactgtttgcaggggaaagctctgggcagcagccaaggggagtggtccggaagggaggggccctggcttctggt

SEQ ID NO: 56 - DNA, 282 bp, Opossum
tgagaacatgctggccccgcagtcccccccgacaccctcagcagctgcctgtgtctgtcagccgcccctcgggggggttgtttgccctctacatcttcctgacagttggctcttctgaaaccagaaaaaaacaacaattcttccttgtcactgaaacccatttgttagatgctgctggggactggcacctttaaccttcttaaagtcaacatttcccttgtcactatgcaggtctggataagaggccaagatggatggaaaacaattgtttgcaaaagaaac

SEQ ID NO: 57 - DNA, 113 bp, Opossum
gtctctggggctgtcttttcatttgcttttttcatttttagacacattccaaactttcagcaaattacagtgtttgccaactggctgtctggggccaaggagtggagttattt

SEQ ID NO: 58 - DNA, 124 bp, Rattus sp.
cctgcccccctacctcaccccaagcttggtctcatttgttgtcttcatttttagacacattccaaacttttcagcaaattacaatgttgccaactggccgtctggggcccaggagagactattt

SEQ ID NO: 59 - DNA, 112 bp, Canis lupus
cccctcaaggctggtctcatttgccttcttcatttttagacacattccaaacttttcagcaaattacagtgtttgccaactggccgtctggggcccaagagagacactattt

SEQ ID NO: 60 - DNA, 612 bp, Homo sapiens
ggtctccagccgagacacggtcgcctttttaaagcccctctctgcttcatgccagccaatgaggacaggctggggcagggctggtcccatgtttccctcagccccggagggagggaaaggggtgtgctcagagcaaaccctccccaaagacttctcctctagctcagcaaacttccaaattgctgctggcactcccaggtgacccagagagagggggcgtgtgaggcaaggcccaagcctgctctccggcacctcccacgtgctggcggctgtggttttcagatatcaaaatgagctccggcttttaattgtctgtctccctgggccctcgggcattctcaaaacaaactgtggacccgctcaacaaagaaaatgtgcagttctgagtgcttcgcggggctgagcccggcctattccatttctcaggccaagttcatttgtcaccccaccaacaaatggcccaaggcgtttctgtttgacccccatagacccagccagaggcaaaaagatgcttttaaagaaatcccattggtcccaactccacaagtctcagcattcattgtcctaaccaagaaagcctctcttggtattctctggaaggcaagagagaga

SEQ ID NO: 61 - DNA, 1086 bp, Mus musculus
aaacctctgaagaagatgctaaaaccaccccagtcacctcccaggacccccccccccactaatgtgcttcttcatcccgtacctttggcgtcatagggatggtggggaggtctgcctccattctccgcccggttcatggtctggttgttctcaggaggaggctcggggtactctccaagccctgggatgacctctgtggcatcattcctgaaggcttgccacccctggccctccacagcacagaaggcagcgtgcacaagtaggcagatgaggcacggggctagtgagggctgcatgctgccaggtggaggagggcactcggtagcacaggctccagtgtccagcctagatacggttgcctttttaaagccccctctgcctcatgccagccaataaggacgggctggagcagggctggtcccatgtttcctcaccctcctcctcacgggggtggggtggggggggtggagaaggggtgtgcttagcgtcagccctcccaagacttctcaagctcagtaaacttccaaattgctgctggcacctcccaggtgacccgaggagtgtgtgttgggggggggggcgttggtgaggcagggacacagccagctctcccagtacctcccacgggcagggcagtcaggctgtggttttcagatatcaaaatgagcttggcttttaattgtctggctccctgggcccctcaggcattctcaaaacaaactgtgggccagcttaacaaaggaaacaagtggggcttctacagaaggcggaatccagcttgaccaaattcctctagccaaggtcatttgttacccctgccagctaatggtcttagttaaaggtgtttgctccccccacccacccagaggcagaaggatgcctttttggggggttgggggcagattctggggactctaacctagggtctcacgcttgctgtgcaagcaccctactattgaagtatgacccagccctgggaaggatgcctttaaaggaatcccattgaccccagctccacattgctcagcacctacaattctgaccaaggagatctctcttgaatttctcttcaaaaccagtgggaga

SEQ ID NO: 62 - DNA, 717 bp, Rattus sp.
agggaggaggagggagggttcacacctgaggtgcaaaaaaaggaggggctggctggcggaagcttcactgagaccgagtgttggaggacgccccagccctggaggaaggctgggaagaaggctgtggtccaccttgccttggtatgctaactgggcaatgaatcagggtcatccacctgcagagcttcaacaaggctccgccacctgccttgacttgtaagtggcctgtcctccttttcatcttctgtcacccacacatcagtgcagctgctctgacagctttacaaggcagaccgaagtcccctcaggaagggggctctgatacctgcttcctaagtcagtttccttcggggcagacaaaaacagctgtggccattgttgggggtgatctatctctcctaccagagctgtggtgttgttagaagctggcccttttgtctgtgacctccagtctcctttccccctgctctggaatgtggactgttaaccctgtctgggccagaaagctgtcctgttgctccagacttgggaagagggcagctcttggacccggattctgagagcatgcagaatggttctcacagcaccacctgctgatgggagttgacgctcgccacctaaagcggagatgccgcctgtccttggaacacttctgccacctgctggacatggaagggaaaaataaggaggccagagagtggccaggctccagatttg

SEQ ID NO: 63 - DNA, 526 bp, Canis lupus
agatgaaggaggagacccacccccgaggaccggggcttcctccctgggaagagccgagcagcccaggtccccatggtattttcactgggcaatgaatcagcacggccccacctgcagagcctctaggaggcctctgccacctgccttgacttgtaagcggccgtgtccatctgcagccgtctgtgcctcctatccaccatccatcactccacacatgaatgcagctgctctgacagttttacaagccagacagaagcccctgcaggaagggccctctctgatacctgtttcctaagtcagtttccttcggggcagacgaaaacagctgtggccattgttggaggtgatctatctctcccaacagagcccctcggtgttgtgagaagctggccctctgtgccccctcccccagtccctgaccctccagcctccttttgcccttgctctggaatgtaggccgttaacccgatcgtggccggacaattgccttctcgccctggatttgggagaaggcaattccgaggct

SEQ ID NO: 64 - DNA, 223 bp, Opossum
tcctttgccgcctgcctcgacttgcaaagtcaacctgtccatggtgcccctccgcctcggtccctctttctacagatcaatcattgaaggtgcttgaacgctgccgccctgacagttttacaagacaggcagaagccccctcaggaaggagtttctctcatacctgtttcccgacttagttacctgcagggaaggtgaaaacagctgttgcttttattggggg

SEQ ID NO: 65 - DNA, 24 bp, Artificial
  synthetic primer sequence
agcaacgcagggcaggagccaaga

SEQ ID NO: 66 - DNA, 24 bp, Artificial
  synthetic primer sequence
tagctggcctctcctgggcgtctt

SEQ ID NO: 67 - DNA, 24 bp, Artificial
  synthetic primer sequence
gggggctgtatggaaaggagacat

SEQ ID NO: 68 - DNA, 23 bp, Artificial
  synthetic primer sequence
cttgagcagtagggccaagccct

SEQ ID NO: 69 - DNA, 24 bp, Artificial
  synthetic primer sequence
tgacaaacaggaaggtggcagggc

SEQ ID NO: 70 - DNA, 23 bp, Artificial
  synthetic primer sequence
cccccaacattcctgtccccttg

SEQ ID NO: 71 - DNA, 24 bp, Artificial
  synthetic primer sequence
tccttgccacgggccaccagcttt

SEQ ID NO: 72 - DNA, 24 bp, Artificial
  synthetic primer sequence
ccccctcatggctggtctcatttg

SEQ ID NO: 73 - DNA, 24 bp, Artificial
  synthetic primer sequence
ccctgagaaacatgcctctgtccc

SEQ ID NO: 74 - DNA, 24 bp, Artificial
  synthetic primer sequence
cttagcaatctgggtgaccctggg

SEQ ID NO: 75 - DNA, 24 bp, Artificial
  synthetic primer sequence
aaactgccaagccccagctggcta

SEQ ID NO: 76 - DNA, 24 bp, Artificial
  synthetic primer sequence
gcccagggctcagaaatgtgtgga

SEQ ID NO: 77 - DNA, 23 bp, Artificial
  synthetic primer sequence
ttcctaccaaggtggctgccacc

SEQ ID NO: 78 - DNA, 25 bp, Artificial
  synthetic primer sequence
ccttcagagaagcaaatggctgggg

SEQ ID NO: 79 - DNA, 22 bp, Artificial
  synthetic primer sequence
cagcagaagatgtcacagcagg

SEQ ID NO: 80 - DNA, 22 bp, Artificial
  synthetic primer sequence
gagctgcatggtaccagccaga

SEQ ID NO: 81 - DNA, 32 bp, Artificial
  synthetic primer sequence
ttggtaccggattgaagtgatccccagctgga

SEQ ID NO: 82 - DNA, 32 bp, Artificial
  synthetic primer sequence
ttgagctccaatctcctgaccttgtgatccgc

SEQ ID NO: 83 - DNA, 31 bp, Artificial
  synthetic primer sequence
ttcccgggcgcttgaacccagtaggtggagg

SEQ ID NO: 84 - DNA, 36 bp, Artificial
  synthetic primer sequence
ttcccgggtaccaagggatggacagaagacaggcag

SEQ ID NO: 85 - DNA, 20 bp, Artificial
  synthetic primer sequence
atgtccaccttgctggactc

SEQ ID NO: 86 - DNA, 20 bp, Artificial
  synthetic primer sequence
gtctgtgggctggtttgcat

SEQ ID NO: 87 - DNA, 24 bp, Artificial
  synthetic primer sequence
agagcctgtgctactggaaggtgg

SEQ ID NO: 88 - DNA, 21 bp, Artificial
  synthetic primer sequence
taggcgttctccagctcggcc

SEQ ID NO: 89 - DNA, 21 bp, Artificial
  synthetic primer sequence
gactggagcctgtgctaccga

SEQ ID NO: 90 - DNA, 24 bp, Artificial
  synthetic primer sequence
cttgagctccgactggttgtggaa

SEQ ID NO: 91 - DNA, 20 bp, Artificial
  synthetic primer sequence
cctctatgccaacacagtgc

SEQ ID NO: 92 - DNA, 20 bp, Artificial
  synthetic primer sequence
ctggaaggtggacagtgagg

SEQ ID NO: 93 - DNA, 19 bp, Artificial
  synthetic primer sequence
tttccatgttgccactcgc

SEQ ID NO: 94 - DNA, 19 bp, Artificial
  synthetic primer sequence
aacggcttgccgttcagca

SEQ ID NO: 95 - DNA, 328 bp, Gallus sp.
aagagctgaggacgcagacagcacggaattggatcatggtcattcaaaccacccgtttaaggcagagacgtgaaaaaaatagccctcagcccacacattgtggcctggcacacagcagcgtccgaagatgaatgcagttgatgagattaatttgttgggcccttacttatcacctgcagacgaggctccgtgcatcatgagctcatttcctggtgctatttcagcctggctccagccaaacagggcaattagctgcggccagcagggggttaaaatgctctcctggttcagccacacgacccccaggctccagcatggccttctgctc

SEQ ID NO: 96 - DNA, 454 bp, Opossum
aaaggaaatgctccagagaatgacctcctcctttccccgtggtccagctctcctgctcgtctctgaattttctgtcttaaggaatccaagtcgcagacagcgcagaatagcactttttcattatcataattcaaattacctgtttaagtcggtcaaatgaaaaaatagccgttcagcccccgcctgtggcctggcagagggaaccttgcgaagcgtgtcctgattcggagctgaagcggtccggcccttccctcccgcctgggctcacagacttcaggctccacgtgccatgagctcatttcctgggggggtgtgagtgtgtgtgtatgtgaatgtgagcttctatttcagcctggctccgtgcaaacagggcaatttactgtgaccaaaaggggcccaaattacattcgtaacatagaaaaatattctccttgccaggccacccatgtgaccg

SEQ ID NO: 97 - DNA, 301 bp, Rattus sp.
  misc_feature (130)..(131): n = a, t, c or g
aggagcctcttggggacccatgccagcaactcccactctctccgaacctttggtggcagacagagcccgaggcacgttttcatgctcataattcaaattgcctgttgaagtcggtcaaatgaaaaaatannaggggagccggagcccagcgctggccgcggcctgggtcctgagctcatttcctgggccgcgcgccgggctatttcagcctggcgctgggcaaacgcgacaattgactgcggcccacgggggcccaaattacaatcggatcccacacaaatagccgccaggcttggcctgc

SEQ ID NO: 98 - DNA, 261 bp, Mus musculus
aggagcctctcgggactcatgccagcgactcccactctctccgagcctttggtggcagacagagcccgaggcacgttttcatgctcataattcaaattgcctgttgaagtcggtcaaatgaaaaaatatgagctcatttcctgggccgcgcgccgggctatttcagcctggcgctgtgcaaacgcgacaattgactgcggcccacaggggcccaaattacaatcggagcccacacaaatagccgccgggcttggcctgccg

SEQ ID NO: 99 - DNA, 260 bp, Canis lupus
aaggtgccaccgcgccccctccgacgtccccacggccgcggctcccacttcgctaggagctctgtcggtggcgcttttccacgatcataattcaaatgagctgttgaagcgggtgcatgaaaaaatatgagctcatttcctggggcgcgcgcgccgggctatttcagcctggcgctgtgcaaacaggacaatttactgcggccaaaaggcgcccaaattacaatcgtatcacacacaaatatccgccacgccaggcctcc

SEQ ID NO: 100 - DNA, 354 bp, Homo sapiens
aagaaagatgctgcagcctccccagtccccatgccagcgctcccacttttctctgagctttcggtggcagacagcgccttgggcactttttcatgctcataattcgaattacctgtttaagtcggtcaaatgaaaaaataccagctccgcccccacgcgggctggccggggcgcctggagcgccagggcggctgcagcgcgctctccgcggccgtcggccctgagctcatttcctggggcgcgcgcgccgggctatttcagcctggcgctgtgcaaacaggacaatttactgcggccaaaagggacccaaattacaatcgtatcacagacaaatatccgccacgccaggtctcc

SEQ ID NO: 101 - DNA, 383 bp, Gallus sp.
aaaccaaacacaaacagccacacaatttcgcacttcacaatggttttccattttagaacaatgtaggctagatccagtgagccattactatttatttattacgaacaagtggaattcaaattaggctgattcgagcctcagcaaccccctcagattggattggtaattaagggactcgtactggtaattatggtttaccatgaaattaccctcccttctcccctctccccccaaaaacaccaaattagccacgagtggaattatatttatcacagcagcccgtgcaggagggactttgtgagtaaatacaaacaaggaggaaaccactgcgccgcgccagcgtgataattagggtttgaaggcaggaattcttgcaggagcca

SEQ ID NO: 102 - DNA, 449 bp, Opossum
aaaggcccagctccctttactctcatgagcactggtcagaaatttaaaataaacagttaaaaaactctgatattaaaaaagttccttccttaaaatattgcaaagggaatagggataattagcaattatcctgcactttattatgagaagacagaattctaattagactgatttgaattccacacacctccacagattgttttgggaattaaggtatcagttgtatgggtaattatggtctaccgttaaattgccgcccccccaaaaaaactgttaaattgtctgtgacgggcttaaatttagcccagactcatgtccaatgaaggctgggcaagtcaatctaaacagccaggaaaccactctagaacctggtgccaagcagtaattagggcctgaggtgaagaaagttttacctgaaacagcagtaatcagctcactggggagggagt

SEQ ID NO: 103 - DNA, 497 bp, Rattus sp.
ggggtctcacagaatccttcaggccagagacccagatcttttccctctcaggggatgtgacctcaaacttcaagcaaatacttgaaaataaaaaccaaattaaaaaaacaaaaccaacctttcctctcttaaaaatattgtgagccagggaagcaatctgcctcaatagtgcattttattatgagatagaatccaaacaggactggtttgggctctgaacacctccacacattgtttggggaattaagggatcagtcgtattggtaattatggtttagcattcaattacccctccagttaaaaaccgttaaattgtctgtgacagggcttaaatttagcccagtctcatgtcctatgaagacttcacgagtcaatacaagccatccggaaaccacaaggtccctgtgccaggtggtaatgaccagttgggatgaggttttttatctgggacatcgggacaagggacaagctgacctacagggcatttatgttggggg

SEQ ID NO: 104 - DNA, 495 bp, Mus musculus
gggatctcacagaatcccccagtccagagacccagatctttttcctctcaggggatgtgatctcaaatttcaagcaaacacctgaaaataaaagccaaattaaaaaacaaaaccaacctttcctgccttaaaatattgtgagccagggaagcaatctgccttgacgctgcgttttattgtgaggtagaattctaactggactggcttgggttcggtacacctccacacattgtttggggaattgagggatcagtagtattggtaattatggtttagcattcaattacccccacagttgaaaaccgttaaattgtctgtgacagggcttaaatttagcccagtctcatgtcctgtgaagacttcacgagtcaatacaagccatccggaaaccacaaggtccctgtgccaggtggtaatgaccagttgggttgaggttttttatctgggacatcgggacaagggacaagctgacctacagggcacttgtgttggggg

SEQ ID NO: 105 - DNA, 528 bp, Canis lupus
ggcctcttgctcttgccaaggggtgaagcactgaggctccagagaaacccaaagtccggagccctgggtcctttccctctcgtggacagtggtccaaaattgaaaacaaacactttaaaataaaagccatattaaaaaacaaacccaactttctgccttaaaatatggtgagccagggggcaattagcaattatgctgtattttattatgagaagatggaattctaattggactgatttgaattccacacacctccacagattgttttgggaattaaggtatcagttgtatcggtaattatggtttaccattcaattactccccccacagaaaactggtaaattgtctgtgacggtgcttaaatttagcccagacccatgtcctatgaagactgcgcgcgaatcaacacgagccgcccggaaaccactgggtgccctgtgccaggcagtaattaggggttgaggtttccaaaattttacctgagacagcagggacaagcccctgggctgcaggatgctcacgtggagg

SEQ ID NO: 106 - DNA, 473 bp, Homo sapiens
ggcgtcttgtcccaagtacagagacctggatcctttcccactcatgtgcaacagcccaaaattaaaaacaaaagccatattaaaaaacaaaaccaactttctgccttaaaatattgtgagccagggggcaattagcaattatgctgtattttattatgagaagatagaattctaattggactgatttgaattccacacacctccacagattgttttgggaattaaggtatcagttgtatcggtaattatggtttaccattcaattacccccccacagaaaactgttaaattgtctgtgacggggcttaaatttagctcagacctatgtcctatgaagactgcgcgagtcaatacaagccatccggaaaccaccgggtgccctgtgccaggcggtaattaggggttgaggtttccaaagttttacctgagacagcagggacaagtgcctgggctgggcgtgctcacgtgggggg

SEQ ID NO: 107 - DNA, 333 bp, Opossum
gcaggtcatgagtcatttcccagcattctggtggtcccagcaattacagagcaagtgagtaattaagcccaaagatcgaggcaaggggaaccccgcatgaggcatgaatgaaaactccgagctggtaaactggtgtgtcagcagccacccctcaccccttactgtgttcccagggcccttgagctgtctacttcaatgatgcacttgccctgtaaccagtgggtgatcagatttttaatctcatagatctctaataacccagtgaaggggggagaggcaggcaggttttgtcaaataggagaagaggaaggagtcctggttgtgaatgtggga

SEQ ID NO: 108 - DNA, 365 bp, Rattus sp.
gcatgctcagccccgctcccctgagaccaggttcccatcactggggtggctggggcagctgccagccaagagtcttaattactataggcttgcagggatctcggctagttccccaccccctacattcccatttctaagtgggaactggatgggcccaccctgggtaaaagggtgtcagcaggcacctgttcacctcttgctgggttcccaggcccctagagccccttggaatagtgaccgtttgccctgcaagcagtgggtgaccaggtttttaatcttggagacccctcagataccagctgggaagtgggatatgtcaaaatgggagggagggagggagagagagcctgtctgtgaatgtcaga

SEQ ID NO: 109 - DNA, 353 bp, Mus musculus
gtctgccaagccctgctcccctgagaccaggttcccatcactggggtggcctgggcagctgccagccaacagcgttaattactacggacttgcagggagctcagcgagcccccaccccctacattcccatttctaagtgggaactgaacgggcccaccatgggtaaaaggggtgtgagcaggcacctgttcatcccttgctgggttcccaggcccctagagctcctggtaatagtgaccggttgccctgtaagcagtgggtgagcaggtttttaatcttggagacccctcagataccagctgggaagtgggatttgtcaaaacgggagggaggtcctgtctgtgaatgtcaga

SEQ ID NO: 110 - DNA, 345 bp, Canis lupus
gcaagccaagtcctgttggacctggacccagattcccatctgaggggtggtctgggcagtggccagccaaaaagtggtaattacttctcgaggtgtggggagctcaggtggcccccaggccctcacctccatgtgggcagtgaatggccctggaagagatggtgtcagcaggcgcctgcccaccccttgctgggttcccaggcccctggagccctctcataataggagccatttgccccataaccagtgggtgaccaagtttttaatcttagaggccccttggatctcagctgggaagtgggatttgtcaaatggggaggggagggcctgtctgggaacgccaga

SEQ ID NO: 111 - DNA, 352 bp, Homo sapiens
gtagggccaagccctgttcagcctgggaccaagttcccatcaacaaggtggtctgggcagtggccagccagaaagcagtaattactgtcgaggtgcagggaccccaggtagggcccccacctcccacctctgtgtgggcagtgaatgggcctgcccctgggtaaggctgtgtcagcaggcgcctgcccaccccttgctgggttcccaggcccctagagccctctcgtaataggagccatttgcgctgtaaccagtgggtgaccagatttttaatcttggagaccccttggatcccaggcgggaagtgggatttgtcaaatggggagaggcggggctgtctgggaatgccaga

SEQ ID NO: 112 - DNA, 223 bp, Opossum
ctgtcctaagctgtcttctctcctggagaggtccaagtttttgtttaccaaactcaaagatgcactttctcttcctgatttcatcaattataaatgtgcttgctactcaccactgtgcaatttgtgaagacctgtggcttgtggtaagagtggctacatccagccttaccacagcggtgaagagatggggtgttggggccagacaaatggaggtggtggtggc

SEQ ID NO: 113 - DNA, 177 bp, Rattus sp.
ctgtatcctgtcccctacccaaggaccagggctttgtttactaaacatcttgggacgagttctcaccgcaaggcaatttgtgttgaccgtggtctgtggtgacactaacaccttccaagctcaccacagccttggcaggggacggggtgggggcagatacagagctgtgggggcggc

SEQ ID NO: 114 - DNA, 225 bp, Mus musculus
ctgcatcctatcccctatcctaacccaagggccagggctttgttcattgagacatcttgggatcagttctcaacccctgatttcatcgattataaatgtgcttgtttctcacggcaaggcaatttgtgttgaccgtggtctgtggtgacactaacaccttccgggctcaccacagcctcggcagagaggagggaggagggccagatacagggctgtggaggtggc

SEQ ID NO: 115 - DNA, 218 bp, Canis lupus
ctctcaagcatccctgcccccactcaagggcccagggttttgtttactaaacaatgagctctcaccccctgatttcatcaattataaatgtgctcgctactcaccacagggcaatttgtgacggactgtggtttgtggtgagagtagcaccatccaagttcaccgcagctgtgagtagagacgagggttagggccagacacagggctgtgggggcggc

SEQ ID NO: 116 - DNA, 225 bp, Homo sapiens
ctctcaagcaccccctcccccacccaaggaccaggtctttgtttactgagcatctcagcgatgagctctcaccccctgatttcatcaattataaatgtgctcgctactcaccacacggcaatttgtgacggactgtggtttgtggtgagagtagcaccatccaagttcaccgcagccgcgagtagagatgagggttggggccagacacagggctgtgggggcggc

SEQ ID NO: 117 - DNA, 125 bp, Gallus sp.; identical to SEQ ID NO: 27

SEQ ID NO: 118 - DNA, 113 bp, Opossum; identical to SEQ ID NO: 57

SEQ ID NO: 119 - DNA, 124 bp, Rattus sp.; identical to SEQ ID NO: 58

SEQ ID NO: 120 - DNA, 131 bp, Mus musculus
cctgccccaccccgccacccccaccccgagcctggtctcatttgttgtcttcatttttagacacattccaaacttttcagcaaattacaatgtttgccaactggccgtctggggctcaggagagactattt

SEQ ID NO: 121 - DNA, 112 bp, Canis lupus; identical to SEQ ID NO: 59

SEQ ID NO: 122 - DNA, 112 bp, Homo sapiens
cccctcatggctggtctcatttgccttcttcatttttagacacattccaaacttttcagcaaattatagtgtttgccaactggccgtctggggcccaggagagatgctattt

SEQ ID NO: 123 - DNA, 371 bp, Opossum
ttttcccttctatttggatgggatcctaatccaataaatgctttggagatttctatggaatagattaatttttactagaaaaaaacataattggtagatgttaaggctattgccctgacaaatctgcctcggctatatttttgaaaaagtaaaagacctgataaacttccccataaatatgccatctgttcagcgagacctgggcgaggagctcccaggaggtttccctgggtgatttattcttcattaataagctctattctatattagtatcagatttatggttctgcctttcgaagatttgggggcattttatctgagtagaatttcaaatgagtgcttgaaacttcgcagcttttctctccgcaggc

SEQ ID NO: 124 - DNA, 455 bp, Rattus sp.
ttagccaagtggtacgggttctctgtgtatgtggagtgtatatttaatcttctatattttggatgagttcctaatccaataaatgcttaggaaatttctatagaacagattaatttttactagaaaaaaatataattggccgatgttaaggctactgccctgacagatcctgccttggccatctatttgaggaagcgagaggcctgatcggcgcgctcataaatatgccatcttcccgtgggccctgagagcactcctcggaggcttcccgccgtgatttattcttcattaataagctctatgctgtattaggatcagatttacgactctgcctctctaatatttctgacatttcatctgaaaggaattccaaaaggaattgtgaaacgttgcccacatcttccctgccagtgttccaaaacctcacatttaagggggctgccggtttggggtgg

SEQ ID NO: 125 - DNA, 441 bp, Mus musculus
ttagccaagtggcgcgggttccctgtgtatgtggagtgtgtatttaatcttctatattttggatgagttcctaatccaataaatgcttaggaaatttctatagaacagattaatttttactagaaaaaaatataattggctgatgttaaggctactgccctgacagatcctgccttggctatctatctgagaaagcgagaggcctcataaatatgccatcttcccctgggccctgagagcactcctcaggggctcctgccgtgatttattcttcattaataagctctatgctgtattaggatcagatttacgactctgcctcactaatatttctgacatttcatctgaaaagaattccaaacgaaatcgtgagacattaccacgtcttccctgctagcgttccaatactgcacacctaaggttgctgctgggatgaggtgg

SEQ ID NO: 126 - DNA, 468 bp, Canis lupus
tcagagcctggcaaaatcttactgtggcgcgtgctgcaggctgtgcatgtgtatgtttaattctctacatttggatatggtcctaatccaataaatgcttaagagatttctctagaatagattaatttttacaagaaaaaaatataattggttgatgttaaggctactgccctgacaaatctgccttggccatatatctgagaaagtaaaagaccccatagactcacacataaatatgccatcctccccacaggccctggtgaagcactccggggagggttccctctgtgatttattcttcattaataagctctatgctatattaggatcagatttatgactctgcctttccaatatttctgacatttcatctgcaaagaattacaaatggtatcttgaaactttgctgcttctcctggctggtgcctgggcactgtccggccaaggggaatcagcgggatggagagg

SEQ ID NO: 127 - DNA, 457 bp, Homo sapiens
tcagggcctctcagagtcctgccgtggtgtgcactgtgtgtgtgtttaattttctacatttggatgtgatcctaatccaataaatgcttaggagacttctatagaatagattaatttttactagaaaaaaatataattggctgatgttaaggctactgccctgacaaatctgccttggccatatatctgagaaggtaaaagacccgctacgcttgcacataaatatgccatcttccccacaggccctggagaagcaccccggggaggtttcccttggtgatttattcttcattaataagctctatgctatattaggatcagatttatgactctgcctttctaatatttctgacatttcatctgaaaagaattacaaatgaaatcttgaaactttgccacttctccctgctagtgctctggcactctgtgtccaaggggagatggtgggctggggaga

SEQ ID NO: 128 - DNA, 357 bp, Gallus sp.
caatggagtccatcacccaatggcttctggaaggcgatgggtaaatcccttcaatcaactccacgccacaatgcatctcttttcttcttttttcccctttaattatggagttggtttaacagcaaatgagaagaaatgtttaaaaggtgacttattaattgcctgtaaattaaaaagcagacggatgctttattgcagttgagagaagttagtaatagtaaggtataatgtcaaatggcaattgacttctttagggttctcttttcttttctaacacataaaggagtgatttatcgcttcctgcagactgagggataaatcatcctggaagtctgaaaactggggctaaataaagat

SEQ ID NO: 129 - DNA, 671 bp, Opossum
tctcttgataaaacctcagatctccatttttcttgagctagtgtagacaaagctggttagtaagttttcacctctatctaggttctcagaagtgtgctggcctccccccaccaaaaaaatccccccatcccattttcagacatgtgacacctgtattattgctaagggttagaaagctcacgaatcaataaaacccattaatgagtgttggtacctcgaaggctacagataaatccgttctactcagtgagttcaatcccataaaacagctctctcctttccattcgagccttcatttgatagaaaatgtggagaaattctaaaaaggtgacttactaattgcctgtaaaataaaaggcagatggaagctttattacagttgaaggaagtcggtaatattaaggtaaaatgtcaaataacaattgattttctccagacataaagatgtgatttatggcttcctagttactacaaacgagaaattatttgaagttctaaaaagtatgaggtgaaataaagattaaatagaagatgaaatcatagaactttctctgggaggtgacttaaatgcccctgggagtgaggattcattcagtcaggtcctgagacagctgaggcagggggatgactaggactagatttagacatgtcccctgggggctttgcagtag

SEQ ID NO: 130 - DNA, 653 bp, Rattus sp.
ttccaaggattagaagttccctcttgtttcttcacttgggcccagagctcagaattgggtggactcctccacctctcccagggtccctgccccctcccaactgccacaggcaggtgacacctgtattattgctaagggttaaaaagccttcaaatcaataaaacccgttaatgagtgttggtacctcgaaggctacagataaatcccctctatcccgtgagttcagtcccataaaacagcgctcccctctccattgcgcgatccatctgacagaaaatgtggagaaatttttaaaaggtgacttactaattgcctgtaaaataaaaggcagatggaagctttattacagtcgaaggaagtcaggaatattaaggtaaaacgtcaaataacaattgattttccttagacataaagggacgatttatggcttcctagttagtacaaatgagaaattatttgaagttctaaaaagtatgaggagaaataaagattaaatagaagatgaaatcatagaggttttttttccccctcccctgggaggtgacttcagtgtccccagagactagaattcatgtagttggggttggggggtaagccaagccagatggggcttggcttagccagtcaagattcaccttggggtatccaaggag

SEQ ID NO: 131 - DNA, 667 bp, Mus musculus
tcccaaggataagaaattctccctcttgttccttcacttgggcccacagagctcagagttgagtggatccctccacctcgcccagcccctgccccctcccaactgccacaggcaggtgacacctgtattattgctaagggttaaaaagcctccaaatcaataaaacccgttaatgagtgttggtacctcgaaggctacagataaatcccctctatcctgagagttcagtcccataaaacagggctcccctctccatcccagcatccatctgacagaaaatgtgaagaaatttttaaaaggtgacttactaattgcctgtaaaataaaaggcagatggaagctttattacagtcgaaggaagtcaggaatattaaggtaaaatgtcaaataacaattgattttccttagacataaagggacgatttatggcttcctagttagtacaaatgagaaattatttgaagttctaaaaagtatgaggagaaataaagattaaatagaagatgaaatcatagagttttttttttttttctgggaggtgacttcagtgtccccagagactagaattcatgtgactgaggggtgggggaggttgtggggagaggtaagttaagacagatggggcttggcttggccagtcaaaattcaccctggggtatccaaggat

SEQ ID NO: 132 - DNA, 629 bp, Canis lupus
ttcaaagggttagaactcatttctcgacatttccagcgtgggcccagagctcagaaccttgtgggcttctttctccctgctcctcccacatccccgccccctcccagggtgacacctgtattattgctaagggttaaaaagcccccgaatcaataaaacccattaatgagtgttggtacctcgaaggctacagataaaccccttctactcagtgagttcaatcccataaaacagctctcccctttcgatcctggcattcatttgatagaaaatgtggagaaatttttaaaaggtgacttactaattgcctgtaaaataaaaggcagatggaagctttattacagttgaaggaagtcgggaatattaaggtaaaatgtcaaataacaattgattttcctcagacataaaggggcgatttatggcttcctagttactacaaatgagaaattatttgaagttctaaaaagtatgagaagaaataaagattaaatagaagatgaaatcatagggatttctctgggaggtgacctcaatgcccctgggggcttgaattcatgtggcggagggcctaggcagctggggcttggtgtggcagcttcaagatttagaggcgaggtgtctctaagggg

SEQ ID NO: 133 - DNA, 634 bp, Homo sapiens
ttcaaagggttagatctcatttctctgcctttctagcttgggcccagggctcagaaatgtgtggactccctcacagcccctcccagcatccctgccccctcccaactgccttgggcaggtgacacctgtattattgctaagggttaaaaagcccccaaatcaataaaacccattaatgagtgttggtacctcgaaggctacagataaatcccttctactcagtgagttcaatcccataaaacagctctcccctttcaatcctagcattcatttgatagaaaatgtggagaaattttaaaaaggtgacttactaattgcctgtaaaataaaaggcagatggaagctttattacagttgaaggaagtcgggaatattaaggtaaaatgtcaaataacaattgattttccttagacataaaggggcgatttatggcttcctagttactacaaacgagaaattatttgaagttctgaaaagtatgaggagaaataaagattaaatagaagatgaaatcatagggatttctctgggaggtgacttcagtgcccctggggactagaattcatgtggccagtggcctagccagctggggcttggcagtttcaagatttagaggcaaggtgtctctgaggag

SEQ ID NO: 134 - DNA, 159 bp, Rattus sp.
ggctatttctaggcaaaaccgcctgatgagatgaaatagctcaattgctgaccattaagtcacgaaggccatggccgttgtaaatctccatctcgccatagccttggcctgcaaggagtgctgggtactgccaggaccagccacctttgtgggaagagc

SEQ ID NO: 135 - DNA, 159 bp, Mus musculus
ggccatttccaggcaaaagcacccgatgagatgaaatagctcaattgctgaccattaagtcatgaaggccatggccgttgtaaatctccaactcagcacagctttggcctgcaaggagtgctcggcactgccaggaccagccacctctgtgggaagagc

SEQ ID NO: 136 - DNA, 155 bp, Canis lupus
agctatttctaggcagaaccataagatgagataaaatagctcaactcctgaccattaagtcatgaaggccatggccatcgtaaatctcagtctctgcggccctctctgtcctgcgtggaagcacagcccagccagtgccagctgctttgatgggc

SEQ ID NO: 137 - DNA, 160 bp, Homo sapiens
ggctatttctaggcaaaactgtctgataagataaaatagctcaactcctgaccattaagtcgtgaaggccatggccatcgtaaatctcatctttccggccctctggcctgcatgcagtgcagcccagccagtcggtggcagccaccttggtaggaagggc

SEQ ID NO: 138 - DNA, 163 bp, Opossum
cccactgtgtttatgactgaaaatgctgcagctttttaaaaagtgtgttaatttcttagagttctgccccagcaattcccagggtttctttttctttaattgaataaataaataatacatgaggcctactggccaaggagaaaatcaggtgctgagcttcctg

SEQ ID NO: 139 - DNA, 150 bp, Rattus sp.
cgtggggtttatggtttcatgagtgtcggagtgataaaaatgtggttatttccagcctcccagagtctgtcttgctctaattgaagaaataaacaatctaccctaaggcatggttagcctaggacagaagtgtgtgggagatctgctggg

SEQ ID NO: 140 - DNA, 156 bp, Mus musculus
cgtggggtttatggtttcatgagtgtcagagtgataaaaaatgttgttatttccagcctgtagcctcccagagtctgtcttgttctaattgaagaaataaacaatctaccctaaggcacggttagcctaggacagaagcgtgtgcgatatctgctg

SEQ ID NO: 141 - DNA, 156 bp, Canis lupus
ccatgaggtttatggtttcatgaatgtcgaagcattttaaaatgtggtaatttcgagtgtgcagcctcccagggcctctctccttttaattgaagaaataaacaatcccccctaaggcatgctcggccacggagaaaggcaggtgcagtgttcagg

SEQ ID NO: 142 - DNA, 157 bp, Homo sapiens
ctgtgaggtttatagtttcatgactgtcagagctttttaaaatgtggtaattttaagtgtgcagcctcccagggtctttcttcttttaattgaagaaataaaccatctcccctaaggcatgcttggcgaaggagaaaggcaggtgcaaggctcacag

SEQ ID NO: 143 - DNA, 276 bp, Opossum
gcctgtgttttccaaaccatttgcctgggagccaacagtggacctatttgggaaagtggcctaacaagataggacaggaactggcaacccagggatttatgtggtgtgggaatggctcacatttccgtcactgtttacacggcagtatttttaaaaaagaaataaatgctagtgaaaaccacagctgtgaattacttataaggggctatgaaagggggtggggaggtaggagtattccctatagagcagagttcccttttcaccaatctgaatgcc

SEQ ID NO: 144 - DNA, 226 bp, Rattus sp.
ggccccccctctttctttccaggtagcaggaccattctgtgggggaaacagctaagcagataggacagcaagctggcgatttatgtggtgtgggaacggctccggtttccctcactgtttacccagcagtattttttttaaaacagaaatcagcatgcagagaaccgcagctgtgaattatgggctctggctgggagggtcacaagggtccctctttcagagtgct

SEQ ID NO: 145 - DNA, 230 bp, Mus musculus
gcccccctttccaactctttcattccaagcagcagggccattatgtggaggaaacagctaagcagataggacagcaagcccgcgatttatgtggtgtgggaacggctccgctttccctcactgtttacccagcagtatattttaaaacagaaatcagcatgtggagaaccgcagctgtgaattatgggctctggctgggagggtcatgagggcacctctttcagagtgct

SEQ ID NO: 146 - DNA, 208 bp, Canis lupus
  misc_feature (170)..(194): n = a, t, c, g, or “blank”
gtgcacttttccagcacttgcctatgaggcagcagtgccattatttttttggtgggggggaccagctaaccagataggacagcaaactggggatttatgtggtgtgggaagagctcggatttccctcactgtttatccagcagtattttttaaaacagaaatcagctgtnnnnnnnnnnnnnnnnnnnnnnnnntccttcatagtgct

SEQ ID NO: 147 - DNA, 235 bp, Homo sapiens
gcctcccttttccagccccttgctttcgaggcagcagtgccattatttggggaaaccagctaaccagataggacagcaaaccggggatttatgtggtgtgggaacagctcaggtttccctccctgtttacccagcagtattttttaaaacagaaatcagcgtgtgggtaaccgcagctgtgagttactagctctggctgtgagggctggggtggggggagtctcttcagagccct

SEQ ID NO: 148 - DNA, 189 bp, Opossum
cccagaaaaccctcctttctctccaacttgtccaggtttgctagcaacatttttagcaaactttcacttgtagatgcaactactcccagccaaattgactagggggaaaaataagtccctacaaatagcaatcactttggaatgaggagagtaattatcttaaattgaggctggtttctgaggggaaaa

SEQ ID NO: 149 - DNA, 374 bp, Rattus sp.
tctttttcctgaagggagggccagatgactcctgaagagcttttgttatcccaggtatggatgacattttaaagagtcaccctgacaccatcatcaaacgttggcagtctcctttcacctccaggtctcctttccgaagaaaagcactgggctggctggcagggggtgtgtaacctagaccatcatccctgggttagtgctggccaacggtccagccacatttttagaaaaatgttcccttttagatttgacttgtcttacttagccagattgacagggtgggggcggctaggtccctatgaatagtgagcacgcgtgagtgaggactcattatcttaaattgaggctggtttctaagaaaaagaaaaggggaa

SEQ ID NO: 150 - DNA, 379 bp, Mus musculus
tcttttcctgaagggagggctagatgactccctgaagagccttccctatcccaggtatggatgacattttaaagagtcacccggcgcacaccatcatcaaaagttggcaacttcgttttacctccgggtctcctttccgaaaggaaaaggcaccggggctggcagagagcatgtaacttagaccgccatccctggtttcgtgccggcacacggtccagccacatttttagaaaaacgttcccttttaaatttgacttgtcttacttagccagattgacagggcgggggtggataagtccctatgaatagtgagcacacgtgaatgaggagactcattatcttaaattgagactggtttctaagaaaaagagaaagggga

SEQ ID NO: 151 - DNA, 335 bp, Canis lupus
tattttagcaaagtctggactaaatgatgccttggggccctcatcgccccaggtttcgaggacattcttcctggttccctccatcacaacaggctctggagtctgtttccatctccagctctctcctgagaagaggggccactcgggctctcttgaaacaagtgacccaaaagatgccccttggtgccctgcaaactgcccagcaacatttttagaaaagtccccttttaaattcaatttgtcttcgccaaatcgaccagggcaaaataagtccctacaaatagcgatcaccagcgaataaggagagtcattatcttaaattgaggctggtttct

SEQ ID NO: 152 - DNA, 353 bp, Homo sapiens
tctttcagtaaagtccagaccaaatgattcttcagggtccttgttcccccaggtttcaatgacactttaaaggtgttgtcgtaaaaggctggggagtctgtttctatgcccccaatctcatttaagaaggggggcatccaggccatcatggaacaggtgacctggaagatgttccttggtccagtttctgcaaactgccccgcagcatttttagaaaatgttcccttttagattcgatttatcttagccaaattgaccagggaaaataggtgcctacaaatagcgatcactggcaaacaaggagagttattatcttaaattaaggctggtttctaacaacaaaaaaaaaccca

SEQ ID NO: 153 - DNA, 298 bp, Opossum
cagaggatatcacactgaaagttcatgcattttaaattgatgtgaaacttcatattttctagtgcaaactatatgttgagtctaattttaacctcagagagctttattattcttttccttttagtgaaagctataatataagtaggaaaaaaaacttgacttgttctctccatttcctgaatatacaattttacaattttctgagatattatttagacttatttttacttttcagcctttccatctccacatttttttccctttaagttccgggtgggatttgcaaaaacatgaaatg

SEQ ID NO: 154 - DNA, 288 bp, Rattus sp.
ggagagatctcccaccggaagtaggcttgctccaaattgatgcctgactggatgccttctgctgtaaactatatgtcgactctaactttagcccgggggagcattcttacttgtttcatgaaagctataatacaggacgagacaactcgcctcatacattccgatttctaaatacacaatttacaattttctgggatattatttaagtttatttttgtgatggagtctcgccatgcccgttgagcgcttatttaagttctgggtggccactggaaaagcctgggatgc

SEQ ID NO: 155 - DNA, 288 bp, Mus musculus
agagagatttcccgctggaagtaggtatgctccaaattgatgcctgactggatgctttctggtgtaaactatatgtcgactctaactttagcccaagcgagcgctcttatttgtttcatgaaagctataatacaggacgagacaactcacctcatacattccgatttctaaatacacaatttacaattttctgggatattatttaagtttattttggtgatggagtctcgccatgcccgttgagcgtttatttaagttctgggtggtcactggaaaagcctgggttgc

SEQ ID NO: 156 - DNA, 295 bp, Canis lupus
gagaggatttcacactggaggtacatgcattccaaattgatacctaactgtatattttttggtgtaaactatatgtcggctctaactttagcctgggggagctttgttatttgtttccctttcatgaaagctataatagaggaagtgaaaaccttgccttgtacattccaattcctaaatacacaatttacaattttcggggatattatttaagtttattttgattctggagtctcaccatccccatgggtagcttatttaggttccgggtggactctagaaaggcctggaatgt

SEQ ID NO: 157 - DNA, 295 bp, Homo sapiens
gagaggattttgcactggaagtacatgcattcaaaatggatacctaagtgtatattttctggtgtaaactatatgtcgactctaactttagcccgggggagctttattatttgtctccctttcatgaaagctataatagaggaagagaaaaccctgcctcgcacattccgattcctaaatacataatttataattttctgggatattatttaagtttattttagttctggatacacaccatccccgtggggtgcttatttaagtatcgggtgggctctggaaaggcctggaatgc

SEQ ID NO: 158 - DNA, 494 bp, Gallus sp.
ggaccctgctggcgactgacagcagcagagccacgggactgctggagaaaactttaatttatttgtacagcatgcatatggcaggtccccacctcctgtttcggtaatgataaatgacaagatgtcgacagttattaattaaagcacccggtgtagcaacgccaaccagaagggctggggaagcaaaatgtgcagcattccttcctcccaccctgagaatggggcccctccaccgttctcgggcacagcagagattaggggatggcagaggtattcctgcacgagccagctccctagtaactgcctgagcacggggcctgaaacattgaaatcctctctcatgccatgtgcagtaattaaacttcggcattccttctgcaggaaatgtttctcctgtgttgttcccacagattgtgctacaacaacaattttagccccgtgaactattaaccaattataaagggattttctcagtccgctttaactgagaatca

SEQ ID NO: 159 - DNA, 682 bp, Opossum
acttttcttccctgaaacttaagaggtggagacacccctcttacacactgcttctgctgaagctcaggaagggagataagactgatctttgaactctagaaaacaatctgggactgactgacatctgttgtctagtaagattgatgggtaaaattttaatttctttgggcggtttgtatttggagggtctccctgactccggcccctatcccttacccccaccagaccccaacctctgattttgctaatgataaatgacaagatgtggacagttattaattaaactgtccatagcgataaggtctgagaatgttatccagcagaagaaatgtgtggcattccttcccacccactccctcctcccttcacttcctccccataccatcccatgagcaggaggcccctttcagatccctggcccagcagaggccggactcacccagcaatatcctccaacaccttggctacctaataacggtctgattcaagggctgagaaatggaaatctctcctggggcctgcacagtaattaacctcagagcattcttgaggcagggaaatgtttctcttgggacattcccacaggccatgtgtaataccaggcttagccccatgaggctaggctgattatgaagggattttcacagttggctctaactgtggatcatgaagatggaggaga

SEQ ID NO: 160 - DNA, 608 bp, Rattus sp.
ggattcccaccggcatggctaaacagccttcccaaaatccgtgggcatgacgtctgggcatggtgtggggctgccgggcacagcctcgtgcgatccgtgcgtgggagtgacagccaggattttaatttgcccatgcaccgtgcttagagctgactttcctggcctctccaacccctcctggtttgctaatgacaagcaatggacatttattaattaaaccgcccacagaggccagtggtaaaaccgaaggccacatgcagcggaggctttcaaagcattcctactgatcatgcctggggcccagcccagagagccaccagggcagccttgggttagtgcaagggtcttcccaggacctgtctactactctgaattctgactaatgaaaaccatctctgcagccatgggataattaaactgcctgcatttctttaaaaaaaaaaaaaaaagtcaaagatcgttcctctagggacattcccacaagctgcagaaggacttgggagttttgagctctcagctaggactgagggattttgaagttggccttgacaggagatttgagggggaggagagtctgggtgggtgggtaggaaatgccttgtaattaa

SEQ ID NO: 161 - DNA, 610 bp, Mus musculus
aaattcccaccagcgtggctaaacagccttcccaaagtccgtgggcgtggcgtctgagacagaagggtatgatgtggggctgccaggcatagccaggtgtgatctgtgcgtgggattgatagccaggattttaattctcccaggcaccgtgcttagagctggatttcctggcctctcagcacctcacctccaacccttcctggtttgctaatggcaaatgacaagcaatggacatttattaattaaactgcccacagaggccagtggtaaaaccgagggccgcatgcagtggaggctttcaaagcactcctactgtttgtgcctggagccaccagggtagccttggcctagcacaagggtcttcccaggactctgaattctgacaaatgaaaaccacctctccagccatgggatgggataattaaactccctgaaaaaaaaaaaaatcaaagatcgttcctctagggacattcccacgagctgcagaaggactggctgggatctttgagctctcagctaggactgagggattttgaagttggccttgactggaggagattggagggggaggagagttgggatgggtgggtaggaaatgccttgtaattaa

SEQ ID NO: 162 - DNA, 689 bp, Canis lupus
acagccttttgctgggatttgacagggaaaaacacaatggttgatggttcccctacaagctgctgcaaagccctgacagaaaaactagattggccactgaacaggaggaaacaacgtggggctgcccaacagcggccaccccacgagattaatgggtaaaactttaatttatccaggcaccttgctttcagagagcctcccctgccctcttggctgcccctaacccaaacccctcctgttttgctaatgataaatgacaagtagtggacatgattaattaaaccgcccccagtagccagtggtaaaacccatggccacaagcagaggagacattcaaagcattcccactgcccctgcccagggcacaggccccaggctccttcctgctctggctagaacaggccccagtggccacggcctcgcacaaagggccttcctaggaactgtctgggctcagctctgtcaaatggaaatcaccaatacgggccacacagtaattaaactcctggcattcttttgacaaaaaatgttcctcttgggacattcccacaggctgttgaaggactgtcggggccctataagctctcagctgattaccaagggatttttgaagttagccttgactgtggatcaaaaggggatgggaattcacggggtgctggtggagatgggaagtgccttgtaacaga

SEQ ID NO: 163 - DNA, 684 bp, Homo sapiens
acagtcctttgctgagatttgacaagggaaacacagtcatagatgaaaccccctacagctgctgcaaagcccttcagaaacatgagattggcctctgaaccagaggaaagtaacgtggggctgtacaacagcagccagcctgtgagactgatggataaagctttaatttatcggtgcaccttgtttggagaaagcttccctgccctcttggctgcccccacctccaacccctcctgttttgctaatgataaatgacaagcagtggacatttattaattaaaccacccgctgtagccattggtaaaacccaaggccacatgcaggggaagcattcaaagcattcctgctgcccctgcccagggcacaggccccagggttatcctttgctggagagagcaggccacggcggccacagcctcacatgatgggccttcctaggatctgtctgggttctggctgataaatggaaatcacctccatgggccacacagtaattaaactcctggcattcttttgacaaaaaaaaagttcctcatgggacattcccacaagctgctgaaggtctgggccctgcaagctcccagctgattaccaaggagttttcgaagttggccttgactgaggatcaaaggaggatgggagttcagggaatgagggtgggggtgggaaatgccttagaattaa

SEQ ID NO: 164 - DNA, 200 bp, Opossum
ccgattagcttcaggctcacaaatcccatatggttagtatatgacattccataaatagcccaaaacacgccctacaatttagcctgtgggttttccccctcatgctaacaggacaggccctcctgggtccccagcctctatgaaagtatcccctttgctggcctctttgtagggcattgtctaccttctttgatcctcac

SEQ ID NO: 165 - DNA, 341 bp, Rattus sp.
ttccttctgtctctgtctgactctcagactattaatacaagcctcatgtctggctgcgccccaagcacacgtgctccctgcaatggagtgccacccctcccagctcagtcctttgtttgttctttctgatgggcgccagcctcatagaccccatagaggtagaatataactttccataaataaactctaacctgacctacaatttagaccttgggctcccccccccctttgtggtaatgggatcatagcccaagctagtctctcctgcatctgcccatcccagaaagcaagccctaggtgtgcttgggtttaggcatcacttggactctctgatcccacat

SEQ ID NO: 166 - DNA, 367 bp, Mus musculus
  misc_feature (239)..(244): n = a, t, c, g, or “blank”
ttccttctgtctctctgtctgcttgactctcagcctattaatacaagccctgtgattaatacaagccccgtgtctggctgtaccccaagcacacgtgctccctgccatagagtaccacccctcccagatcagcctgttgtttgttctttctgatgggcgccagccttgtagaccccatggaggtagaatataactttccataaataaactctaacctgacctacaatttggaccttggnnnnnnttccctctttgtggtaatgggatcacagcccaacctagtctctcctgcatctgtctgtcccagaaagcaagtcctaggtgtgtttgggtttgggcatcacttggactctctgatgcccctcat

SEQ ID NO: 167 - DNA, 342 bp, Canis lupus
ttccctctgcccctctgtctgactcccaagactattaatacaaaccccagtctggctgtgccaccagaacatgtgctccccacagcagaatgccacccctcccagctgagcctgttgtctgttccgtttctgatcggcgccaggctcatagaccccatgtaggtagaatataactttccataaataacccctagcctgacctacaatttaggctttgggtttttttcccctcgtggtaatgggattatagcctgggctgatccctcctgggtcttggggtcccagaaagcagaccctaggtgtatcgggctgggcattgcttggaattgccggttcccccat

SEQ ID NO: 168 - DNA, 344 bp, Homo sapiens
ttccttctatccctctgtctgtcttactctcagactattaatacaagccctgagtctggctgtacccccagaacatgtgccccgccccctacaacaaaatgctgcccctcccagctaggtctgttgtttgttccttttctgattggcgccaggcttatagaccccatgtaggtagaatataactttccataaataacctctaacccgacctacaatttagccttcaggtttttttccccctcgtggtaatgggattgcagcctgggctgatccatcctgtatcttcaggtcccagaaagcagaccctaggtttggacattgcttggaattcctggtacccccat

SEQ ID NO: 169 - DNA, 394 bp, Opossum
ccccttggggcttggcagcagggctatgaaatgggatgtgagcagtggggtatatagagaagtggtaggtggttagggagcagtgctcttggtggtgagaacatgctggccccgcagtcccccccgacaccctcagcagctgcctgtgtctgtcagccgcccctcgggggggttgtttgccctctacatcttcctgacagttggctcttctgaaaccagaaaaaaacaacaattcttccttgtcactgaaacccatttgttagatgctgctggggactggcacctttaaccttcttaaagtcaacatttcccttgtcactatgcaggtctggataagaggccaagatggatggaaaacaattgtttgcaaaagaaacttctttagtgaactcaa

SEQ ID NO: 170 - DNA, 550 bp, Rattus sp.
  misc_feature (209)..(209): n = a, t, c, g, or “blank”
tgtgatggaaggagcccacaggaggggtgggctaaggggaagatggggctgtcagctccttaggaagccctggcagtaggcaagctgtaacaagatccctgttgggccatccttctcacatctggctcagggtccccatcccagggaggccccgtggccctttgccctgggaaggggtgggcactgggtcacggtgggggggggtgggngaggtcanaaagggcatggggcggggcacagcagggtgctatgtgctgggaacacagggcctggcaccccagtgccccagcagctgctggcgtctgtcaacccccctgggggcaccacaaggccaccacctctacatcttcctgacaggcccctcttctgaggccaggaaaaaacaacagccccctcctacagcaacccatttgtcagctgaaggctgtccctgcacctttaacctcctcaaagtcagcagttccctgtcaaggccctgtggggccaggagacaaagatggatggaggtgactgtttgcaggggaaagctctgggcagcagccaaggggag

SEQ ID NO: 171 - DNA, 556 bp, Mus musculus
tgagatggaaggagcccacaggaggggtgggctgaagggaagatggggctgtcagctccttaggaaacccttggggcctgacctggcaaactgtaaccagatccctgttgggctgtcctcctcacatctggctcagggtccccattgcagggaggcccccttggccctttgccctgggaaggggtgggcaccgggtcacggtggggtgggtggggaggtcagaaagggcatgaggcgggggcacagcggggtgctatgtgctgggaacataggcctggcaccccggtgccccagcagctgctggcgtctgtcagccccccctaggggcaccacaaggccgccacctctacatcttcctgacaggtccctcttctgaggccaggaaaaaacaacagccccctcccacagcaacccatttgtcagctgaaggccggccctgcacctttaaccttctcaaagtcagcagttccctgtcaaggccctgtggggccaagagacaaagatggatggaggtggctgtttgtagaggaaagctcttggctgcagccaaggggag

SEQ ID NO: 172 - DNA, 498 bp, Canis lupus
  misc_feature (177)..(219): n = a, t, c, g, or “blank”
tgggggctctagaaggatccacaggaggctgggaggaagccctggcctgtcagcttccccaggcaaaccttcccgggggcctggccccagcggctgtaaccggactcctgctccggcctccgcagctggccctggggaggcccagcgcggggaggcccagccgcctcctcgggccgnnnnnnnnnnnnnnnnnnnnnnnnnnnnnnnnnnnnnnnnnnnggcgcccgcgcagctgcccgcgtctgtcannnnnnnnnnnnnnnnnnnnnnnnnnnnntggcctctacatcttcctgacaggcccctcttcggaggccaggaaaaaacaacagtccctcccctcgcggcagcccatttgttagatgaaggccgggcaccggcacctttaacctcctcaaagtcagcatttccccctcccggccccgcgggccccagacagagatggatggaggcggctgttggtggaagaaaggcccgtggctgcgcggccaaggggag

SEQ ID NO: 173 - DNA, 564 bp, Homo sapiens
tgggggttctggaaggaatccgtgggaggctgggaggaagatctggcttgtcagcttccctaggaaaaccttcccctgggctggccgcaggctgtaaccggattcctgctccacctctgcatctggcccagggacctcatggcagggaggcccagcgcctggccctttgcccctggacggggtgggccctgggtcatggtggggtgggtggggaggtcaggagggccatggggagggggcgcggtggggtgctttgccctgagaacacaggcctctggcaccccggagcccccggcagctgctggcgtctgtcagccaccttgcggggcgcggccgggggctgctggcccctacatcttcctgacaggcccctcttctgaggccaggaaaaaacaacaacagttcctcccctcacggcaacccatttgttagatgaaggccgggcaccagcacctttaacctcctcaaagtcagcgtttccctgtcaaggccccacagggccagagacagagatggatggaaggagctgtgtgtcgaaaaagccctgtggcctcatgaggagag

SEQ ID NO: 174 - DNA, 231 bp, Gallus sp.
actctctgcagctgctgctgtcactcagcggcccagcgggggcttgtttgccctctgcatcttcctgacacttcattcctgaaggaagcaaaaacaacagccctcccctcttgcccaagcccatttgttggacgaggcagccacctttaacctctgcagagtcaacatctcttctgccagaccccaggctgggcgggaaacataggatggatgggtgcaataggtttcaaa

SEQ ID NO: 175 - DNA, 633 bp, Opossum
agtccttggagcactttctccttttttaaatccccctttgccgcttcaccagccaatggggttacagacagagaaaggccagccccacatttcacttggggggaggggtggtaggaaatcgagagggaaggcaaaggggtgtgttcagagccaacccacttcaccacttttcttcccaggctgtctaaacttccaaatagctccagtctcttcccaggtagtttggaatgtctaggttcatgcctggcagatccataaggaagaggggtccttttcagcgtccagctctttttcggttattcaaaagctgctttcctcctcagacggatgacgtctctggacccctttatctgtctcaaaacaagctggatcccagtttaacaagcaaacttcccaagtctccttcaggaagctttgccgcctgcctcgacttgcaaagtcaacctgtccatggtgcccctccgcctcggtccctctttctacagatcaatcattgaaggtgcttgaacgctgccgccctgacagttttacaagacaggcagaagccccctcaggaaggagtttctctcatacctgtttcccgacttagttacctgcagggaaggtgaaaacagctgttgcttttattggggg

SEQ ID NO: 176 - DNA, 990 bp, Rattus sp.
agtccccagcccagatacggtcgccttttaaaagccccctctacctcatgccagccaataaggacaggctggaacagagctggttccatgtttcctcaccctcctccccatgggggtggggagaagggggtgtgcccagtgtcagccctccccaagacttctcaagctcagcaaacttccaaattgctgctggcactcccaggtgacccgaggaatggggtgggggtggggggggttggtgaggcagggacacagccagctctcccagcacctcccacaggcagacggctgtggttttcagatatcaaaatgagctcggcttgtaattgtctggctccctgggcccctcaggcattctcaaaacaaactgcgggctagcttaacaaaggaaacaagtggggcttctacagaaggcagaatccgagggagggttcacacctgaggtgcaaaaaaaggaggggctggctggcggaagcttcactgagaccgagtgttggaggacgccccagccctggaggaaggctgggaagaaggctgtggtccaccttgccttggtatgctaactgggcaatgaatcagggtcatccacctgcagagcttcaacaaggctccgccacctgccttgacttgtaagtggcctgtcctccttttcatcttctgtcacccacacatcagtgcagctgctctgacagctttacaaggcagaccgaagtcccctcaggaagggggctctgatacctgcttcctaagtcagtttccttcggggcagacaaaaacagctgtggccattgttgggggtgatctatctctcctaccagagctgtggtgttgttagaagctggcccttttgtctgtgacctccagtctcctttccccctgctctggaatgtggactgttaaccctgtctgggccagaaagctgtcctgttgctccagacttgggaagagggcagctcttggacccggattctgagagcatgcagaatggtt

SEQ ID NO: 177 - DNA, 1031 bp, Mus musculus
agtgtccagcctagatacggttgcctttttaaagccccctctgcctcatgccagccaataaggacgggctggagcagggctggtcccatgtttcctcaccctcctcctcacgggggtggggtggggggggtggagaaggggtgtgcttagcgtcagccctcccaagacttctcaagctcagtaaacttccaaattgctgctggcacctcccaggtgacccgaggagtgtgtgttgggggggggggcgttggtgaggcagggacacagccagctctcccagtacctcccacgggcagggcagtcaggctgtggttttcagatatcaaaatgagcttggcttttaattgtctggctccctgggcccctcaggcattctcaaaacaaactgtgggccagcttaacaaaggaaacaagtggggcttctacagaaggcggaatccgagggagggttcacacctgaggtgcaaaaggaggaggggctggcggaagctgcactgagacagaaggttggaggactccccagccctggaggaagactgggaaggaggctgtggtccgcctagcccgctgtggtatgctaactgggtaatgaatcagtgtcgtccacctgcagagcttctgcaaggctctgccacctgcctcgacttgtaagtggccggtccatctgtggcgatctgcctgggtcctccttttcatcttctgtcacccacacatcaatgcagctgctctgacagctttacaaggcagacagaagtcccctcaggaaggggactctgatacctgcttcctaagtcagtttccttcggggcagacaaaaccagctgtggccattgttgggggtgatctatctctcctaccagagctgtgaagttgttagaagctggcccttttgtctgtgacctccagtctccttttcccctgctctggaatgtggactgttaaccctgtctgggccagaaagctttcctattgctccagacttgggagagggcagctcctgggcccgggattctgtgagcaccataatggtt

SEQ ID NO: 178 - DNA, 1013 bp, Canis lupus
ggtccccagcctgggacctgtttgcctttttaaagccccctccgcctgatgccaaccaatgaggacaggctggggcagggctggtcccatgtttctctcagcccagggggagggaacggggtgtgctcatggcaagccctccccgaagacttctcaggcacagcaaactcccaaattgctgctggtgctcccaggtgacctagagggagggggagtgtgtgtgtgaggcaagcctgccctccagcacctcccacgtgcaggcggctgtggtttttagatatcaaaatgagctccagctcgtaatctttacctccttgggccccttgggctttctcaaaaacaaacttcggaccagcttcacgaagaaaacgagagctctgagcccacagggctgagcccggagaaaggttcacacctgaggtgcaggaaaagaaaggagagcaagcggaagctttcactgcagatgaaggaggagacccaccccgctcctggccccgaaggaggctgggaagagggctgcagcccaggtccccttggtattttcactgggcaatgaatcagcacgcccccacctgcagagcctctaggaggcctctgccacctgccttgacttgtaagcggccgtgtccatctgcagccgtctgtccctcctatccaccatccatcactccacacatgaatgcagctgctctgacagttttacaagccagacagaagcccctgcaggaagggccctctctgatacctgtttcctaagtcagtttccttcggggcagacgaaaacagctgtggccattgttggaggtgatctatctctcccaacagagcccctgggtgttgtgagaagctggccctctgtgccccctcccccagtccctgaccctccagcctccttttccccttgctctggaatgtaggccgttaacccgatcgtggccggacaattgccttctcgccctggatttgggagaaggcaattccgaggctccatccctggagctggcgcgatgggc

SEQ ID NO: 179 - DNA, 998 bp, Homo sapiens
ggtctccagccgagacacggtcgcctttttaaagcccctctctgcttcatgccagccaatgaggacaggctggggcagggctggtcccatgtttccctcagccccggagggagggaaaggggtgtgctcagagcaaaccctccccaaagacttctcctctagctcagcaaacttccaaattgctgctggcactcccaggtgacccagagagagggggcgtgtgaggcaaggcccaagcctgctctccggcacctcccacgtgctggcggctgtggttttcagatatcaaaatgagctccggcttttaattgtctgtctccctgggccctcgggcattctcaaaacaaactgtggacccgctcaacaaagaaaatgtgcagttctgagtgcttcgcggggctgagcccggagaaggattcacacctgaggtgcaagaagagaaaggaaggccagaggaatctttcattgaagacagattgtttgaggacaagcagctcctgtgaagaggctgcccagatcctctggtattttaactgggcaatgaatcagcatgccccacctgcagagcctctgcaagtcctttgccacctgcctcaacttgcaagtcgctgtgtccatctgcagccgactgtcctcctttccatcatccatcatccgacacatgaatgcagctgctctgacagttttacaagccagacagaagccccctcaggaagggccctctctgatacctgcttcctaagtcagtttccttcagggcagatgaaaacagctgtggccattgttgggggtgatctatctctcccagcagagccggtagtgttgtgagaagctggccctccgcgccacctccccttgtctatgaccctccagcctccttttcccttgctctggaatgtggactgttaaccctctcatgaccagacgactgccttcttgcaatggacttgggagagagcagttctgatgcctgggttccttgagcaggtaggatggat

(19) United States
(12) Patent Application Publication
(10) Pub. No.: US 2009/0017008 A1
(43) Pub. Date: Jan. 15, 2009

(54) Compositions and methods for altering bone density and bone patterning

(75) Inventors: Gabriela G. Loots, Oakland, CA (US); Edward M. Rubin, Berkeley, CA (US)

Correspondence Address: LAWRENCE BERKELEY NATIONAL LABORATORY, ONE CYCLOTRON ROAD, MAIL STOP 90B, UNIVERSITY OF CALIFORNIA, BERKELEY, CA, 94720, US

(73) Assignee: The Regents of the University of California, Oakland, CA (US)

(21) Appl. No.: 11/953,796

(22) Filed: Dec. 10, 2007

Related U.S. Application Data
(63) Continuation of application No. PCT/US2006/022455, filed on Jun. 9, 2006.
(60) Provisional application No. 60/689,782, filed on Jun. 10, 2005.

(51) Int. Cl. A61K 39/395; C12N 5/06; A01K 67/027; C12N 15/00; A61P 43/00; C12Q 1/68; C12N 15/11; A61K 31/70

(52) U.S. Cl. 424/130.1; 435/375; 514/44; 800/13; 800/18; 536/23.5; 435/320.1; 435/325; 435/6

(57) ABSTRACT

By exploiting cross-species sequence comparisons with in vitro and in vivo enhancer assays we were able to identify enhancer elements that drives human SOST expression in the adult mouse skeleton, and discovered a novel function for sclerostin during limb development. The enhancer elements and reagents described in the present invention facilitate the methods for development of products and methods to increase the mineral content of bone, which can consequently be utilized to treat a wide variety of bone related conditions, including, osteopenia, osteoporosis, fractures and other disorders in which low bone mineral density are the main cause of the disease as well as sclerosteosis, Van Buchem disease and other related disorders of the skeleton. Furthermore, the present invention provides enhancer elements and reagents useful for bone pattering and growth, limb development, and the formation of individual bones

### CROSS REFERENCE TO RELATED APPLICATIONS

This application is a continuation under 35 U.S.C. § 120 of International Application No. PCT/US2006/022455, entitled Compositions and Methods for Altering Bone Density and Bone Patterning, filed on Jun. 9, 2006 under the Patent Cooperation Treaty, which was published by the International Bureau in English on Dec. 21, 2006 with International Publication Number WO/2006/135734, which designates the United States and which claims the benefit of U.S. Provisional Patent Application No. 60/689,782, filed on Jun. 10, 2005. Each of the above reference applications is incorporated in its entirety by reference herein.

### STATEMENT OF GOVERNMENTAL SUPPORT

This invention was made with government support under Contract No. DE-AC02-05CH11231 awarded by the U.S. Department of Energy and Grant No. P60HL20985 awarded by the National Institutes of Health. The government has certain rights in this invention.

### REFERENCE TO SEQUENCE LISTING, TABLE, OR COMPUTER PROGRAM APPENDIX

This application incorporates by reference the attached sequence table and sequence listing, found in paper and computer-readable form.

### BRIEF DESCRIPTION OF THE TABLES

Table 1 provides a sequence alignment for chicken, opossum, rat, mouse, dog and human ECR1.

Table 2 provides a sequence alignment for chicken, opossum, rat, mouse, dog and human ECR2.

Table 3 provides a sequence alignment for opossum, rat, mouse, dog and human ECR3.

Table 4 provides a sequence alignment for opossum, rat, mouse, dog and human ECR4.

Table 5 provides a sequence alignment for chicken, opossum, rat, mouse, dog and human ECR5.

Table 6 provides a sequence alignment for opossum, rat, mouse, dog and human ECR6.

Table 7 provides a sequence alignment for chicken, opossum, rat, mouse, dog and human ECR7.

Table 8 provides a sequence alignment for rat, mouse, dog and human ECR8.

Table 9 provides a sequence alignment for opossum, rat, mouse, dog and human ECR9.

Table 10 provides a sequence alignment for opossum, rat, mouse, dog and human ECR10.

Table 11 provides a sequence alignment for opossum, rat, mouse, dog and human ECRA.

Table 12 provides a sequence alignment for opossum, rat, mouse, dog and human ECRB.

Table 13 provides a sequence alignment for chicken, opossum, rat, mouse, dog and human ECRC.

Table 14 provides a sequence alignment for opossum, rat, mouse, dog and human ECRD.

Table 15 provides a sequence alignment for chicken, opossum, rat, mouse, dog and human ECRE.

Table 16 provides a sequence alignment for SOST promoter in opossum, rat, mouse, dog and human.

### BRIEF DESCRIPTION OF THE SEQUENCES

SEQ ID NOS: 1-15 refer to human ERC sequences. The sequences for ERC1, ERC2, ERC3, ERC4, ERC5, ERC6, ERC7, ERC8, ERC9, ERC10, ERCA, ERCB, ERCC, ERCD, ERCE have been provided.

SEQ ID NO: 16 refers to the human SOST gene, GenBank Accession No: NM_{—}025237.

SEQ ID NOS: 17-59 refer to ECR sequences in other organisms including dog, opossum, rat, mouse, and chicken.

SEQ ID NO: 60-64 refer to genomic SOST sequences in human, mouse, rat, dog, and opossum.

SEQ ID NOS: 65-80 set forth primers for amplifying ERC enhancers.

SEQ ID NOS: 81-86 set forth primers for generating transgenic mice as in Example

SEQ ID NOS: 87-92 set forth SOST RT-PCR primers.

SEQ ID NOS: 93-94 set forth primers.

SEQ ID NOS: 95-179 refer to individual sequences of each organism found in the alignments of Tables 1-16.

### BACKGROUND OF THE INVENTION

1. Field of the Invention

The present invention relates to compositions and methods to altering bone density, growth and mineral content of bone and bone patterning.

2. Related Art

Deleterious mutations in distant regulatory elements postulated to dramatically impact human development and health have been minimally explored. This problem is in large part due to the fact that there are no simple ways to discern regulatory elements from nonfunctional sequences or to ascertain whether mutant phenotypes are caused by regulatory mutations. Among Mendelian disorders associated with noncoding mutations only a few cases are described that clearly link alterations in distant cis-acting regulatory regions to the cause of the disease, and these documented cases predominantly correspond to large chromosomal aberrations. Structural variation in the human genome described as large-scale polymorphisms has been recently shown to be more common than previously anticipated, therefore the extent to which large noncoding duplications and deletions impact human biology remains a largely unanswered question. In this study, we demonstrate that a very important skeletal dysplasia, Van Buchem (VB) disease, associated with a large noncoding deletion is caused by the removal of a bone-specific distant enhancer element.

Van Buchem disease (MIM 239100) is a homozygous recessive disorder that maps to chromosome 17p21 and results in progressive increase in bone density. The accumulation of bone mass gives rise to facial distortions, enlargement of the mandible and head, entrapment of the cranial nerves, increase in bone strength, and excessive weight. Sclerosteosis (MIM 269500) is a cranio-tubular hyperosteosis that is phenotypically indistinguishable from Van Buchem disease (VB) except that it is more severe and occasionally displays syndactyly of the digits, a trait absent in VB patients.

The genetic factors that contribute to susceptibility to bone loss are extremely heterogeneous, therefore murine models that affect bone development and growth can provide invaluable insights into the molecular mechanisms of progressive bone loss in humans. Human genetic diseases of the skeleton such as sclerosteosis and Van Buchem disease provide a starting point for understanding the modulation of anabolic bone formation, and ultimately have the potential to identify key molecular components that can be used as new therapeutic agents to treat individuals suffering from bone loss disorders.

Thus, there is a need in the art to identify compositions and methods for modulating bone formation. The present invention satisfies these and other needs.

### BRIEF SUMMARY OF THE INVENTION

The present invention provides compositions and methods for modulating bone density, e.g., by modulating differentiation, function, and proliferation of cells of bone lineage (e.g., mesenchymal cells, osteoblasts, osteoclasts, and osteocytes).

One embodiment of the invention provides methods of modulating proliferation of a cell of bone lineage. The method comprises contacting the cell with a composition that modulates the function of a SOST regulatory element, wherein the regulatory element is selected from the group consisting of: ERC1, ERC2, ERC3, ERC4, ERC5, ERC6, ERC7, ERC8, ERC9, ERC10, ERCA, ERCB, ERCC, ERCD, ERCE, and combinations thereof. In some embodiments, the regulatory element comprises a sequence selected from SEQ ID NOS: 1, 2, 3, 4, 5, 6, 7, 8, 9, 10, 11, 12, 13, 14, and 15. In some embodiments, the regulatory element is an enhancer (e.g., ERC5). In some embodiments, the ERC5 comprises the sequence set forth in SEQ ID NO: 5. In some embodiments, the composition is selected from a small molecule, an antibody, and an aptamer. In some embodiments, the cell is in a vertebrate (e.g., a mammal including rodents such as a mouse, a rat, a guinea pig or rabbit; an avian such as a chicken, a turkey or a duck; an amphibian such as a frog or a toad, a primate such as a chimpanzee, a monkey, or a human). In some embodiments, the vertebrate has been diagnosed with a disease or disorder associated with aberrant bone density. In some embodiments, the bone density of the vertebrate is increased following contact with the composition that modulates the enhancer of SOST. In some embodiments, the disease or disorder is selected from: osteopetrosis, osteopenia, osteosclerosis, craniotubular hypertoses, Van Buchem's disease, and osteoporosis. In some embodiments, the composition inhibits the function of the SOST regulatory element. In some embodiments, the composition stimulates the function of the enhancer of SOST regulatory element.

The invention provides homozygous knockout non-human animals that are lacking any or all of the SOST regulatory elements described herein. In some embodiments, the animals down regulate expression and production of SOST protein. These animals will have decreased (or lack) SOST levels and thereby modulating bone density levels. This invention also includes recombinant vectors and DNA targeting constructs, such as the one used by the inventors to delete mouse VB deletion and was built using PCR products and primers made from SEQ ID NOS: 81-84. In a preferred embodiment, the knock-out (transgenic) animals are mouse models exhibit limb defects which can be studied to understand bone patterning processes.

The invention also provides non-human animals that over-express any one or combinations of the human SOST regulatory elements described herein. The over-expression of human SOST under the control of its own proximal promoter elements in concert with the downstream VB region negatively modulates adult bone mass. In development, the over-expression of an enhancer elements to increase SOST levels in normal animals or in animals missing the VB region can be used to affect bone, limb and digit development.

This invention also provides non-human animals for further animal studies by pharmaceutical companies to study human or mouse SOST enhancer and other regulatory elements. Animal studies that explore the regulation and expression of human or mouse SOST, its interaction with other related proteins, particularly upstream or downstream members of the pathways specific to SOST, production of antibodies for proteins physically interacting with mutant and wild-type SOST regulatory elements, and further in vivo study of SOST and its regulatory elements. For example, wild-type mice or rats may be exposed to various test ECR5 inhibitors to determine the SOST lowering effect of the test substance to resemble effects observed in Van Buchem's disease or other bone related diseases. Similarly, ovarectomized or osteopenic mic or rats may be exposed to various test ERC5 inhibitors to produce bone growth for studying ostepenia and osteoporosis.

Another embodiment of the invention provides transgenic non-human animals having cells comprising a chromosomally incorporated transgene comprising a recombinant polynucleotide encoding sclerostin (SOST) and a recombinant polynucleotide encoding MEOX1 operably linked to a regulatory region comprising a sequence set forth in any one of SEQ ID NOS: 1-15 and 17-59, wherein the animal exhibits altered bone mineral density, limb deformities, and SOST is expressed embryonically and in the adult bone, liver, brain, lung, heart and kidney tissues. In some embodiments, the transgenic animal is a mouse. In some embodiments, all of the cells in the mouse comprise the chromosomally incorporated transgene.

A further embodiment of the invention provides transgenic non-human animals having cells comprising a chromosomally incorporated transgene comprising a recombinant polynucleotide encoding sclerostin (SOST) and a recombinant polynucleotide encoding MEOX1 operably linked to a regulatory region, wherein the 52 Kb Van Buchem deletion region has been deleted from the regulatory region, wherein the animal exhibits altered bone mineral density, limb deformities, and SOST is expressed embryonically in the heart and kidney tissues. In some embodiments, the transgenic animal is a mouse. In some embodiments, all of the cells in the mouse comprise the chromosomally incorporated transgene.

Another embodiment of the invention provides isolated polynucleotides for modulating SOST expression, the nucleotide having 95% identity to at least one sequence selected from SEQ ID NOS: 1-15 and 17-59. In some embodiments, the invention provides expression vectors comprising the polynucleotides operably linked to a gene selected from Lac-Z, β-gal, GFP, cre-recombinase, and human SOST. In some embodiments, the invention provides host cells and transgenic non-human animals having cells comprising the expression vector comprising the SOST-specific regulatory elements operably linked.

In another embodiment, a method to determine the genetic status of an individual, the method comprising: detecting a variation in the sequence of at least one SOST regulatory element wherein the regulatory element is selected from the group consisting of: ERC1, ERC2, ERC3, ERC4, ERC5, ERC6, ERC7, ERC8, ERC9, ERC10, ERCA, ERCB, ERCC, ERCD, ERCE, and combinations thereof. In a preferred embodiment, the elements have the sequence of one of SEQ ID NOs: 1-15.

### BRIEF DESCRIPTION OF THE DRAWINGS

FIG. 1. Generation and Characterization of Van Buchem transgenic mouse models. (A) A 158 kb human BAC ( [symbol] ) spanning SOST and MEOX1 was engineered using in vitro BAC recombination in E. coli (Lee et al. 2001) by deleting the 52 kb noncoding region missing in VB patients ( [symbol] ). Three independent transgenic lines were generated for each BAC construct. Human SOST expression was analyzed by rtPCR in adult tissues (B) and embryonic tissues (C) of [symbol] and [symbol] transgenic mice. Embryonic expression was used to quantify transgene expression levels in independent transgenic lines (D).

FIG. 2. SOST transgenic expression negatively impacts bone parameters. (A) Body weight measurements of 5-month-old male mice (non-tg=13, SOST^{wt}=15, SOST^{wbΔ}=14). (B) Bone mineral density in the tibia, femur and lumbar spine as evaluated by DEXA. (C) Bone volume, trabecular number, thickness and separation as evaluated in the cancellous bone compartment of the proximal tibia metaphysis by μCT. (Mean +/−SEM; * p<0.05 versus non-tg).

FIG. 3. Human SOST dose effect on bone metabolism in the proximal tibia metaphysis of 5-month-old male mice (non-tg=5, SOST^{wt}=7, SOST^{wt/wt}=4). (A) Bone volume and (B) bone formation rates as determined by μCT scans and histomorphometric analysis respectively. (Mean +/−SEM; * p<0.05 versus non-tg; x p<0.05 versus SOST^{wt}). (C) Cancellous bone compartment of non-transgenic and SOST^{wt/wt} mice. (D) Fluorochrome marker uptake at site of active mineralization of bone matrix laid down by osteoblasts in wildtype and transgenic mice at the interface between endocortex and cancellous bone.

FIG. 4. Embryonic SOST expression and limb deformity in [symbol] and [symbol] transgenic mice. Embryonic SOST expression was predominantly detected in the developing limb bud during E9.5 to E12.5, as visualized by whole mount in situ hybridization using mouse SOST probes (A). μCT scans of defective limbs overexpressing human SOST (B). Skeletal preps showing how the bones of the forelimb (hand, wrist and arm) are affected at elevated human SOST levels (C).

FIG. 5. Enhancer activity of evolutionarily conserved noncoding sequences from the Van Buchem deletion region. (A) Human/Mouse genomic alignment generated using zPicture alignment engine (URL:<http://zpicture.dcode.org/>). Exons are in blue, untranslated regions in yellow, repetitive elements in green and noncoding sequences in red (intragenic) or pink (intronic). Seven highly conserved elements (≧200 bp; ≧80% ID; ECR2-8) within VBA and the promoter region were tested in rat-osteosarcoma (UMR-106) and kidney cells (293) for the ability to enhance luciferase expression from the SV40-promoter (B) or human SOST promoter (C). ECR5 activates the human SOST promoter in rat osteosarcoma cells (C), and drives the hsp68 promoter in the skeleton of E14.5 mouse embryos (D). Detailed transgenic expression of ECR5 in the skeleton showing its specificity to bone and kidney (E).

FIG. 6. Genomic alignment of evolutionarily conserved noncoding sequences from the Van Buchem deletion region using Mulan alignment engine (URL:<http://mulan.dcode.org/>). Exons are in blue, untranslated regions in yellow, repetitive elements in green and noncoding sequences in red (intragenic) or pink (intronic). ECR5 shown to have in vivo activity is shown in purple, and the Van Buchem deletion region is boxes in purple also.

### DETAILED DESCRIPTION OF THE PREFERRED EMBODIMENT

Introduction

The present invention is based on the discovery that the regulatory elements ERC1-10 and ERCA-E modulate expression of sclerostin (SOST). One embodiment of the invention is based on the identification of ERC5 as a specific enhancer of SOST.

Using cross-species sequence comparisons coupled with transgenic analysis, the present invention identifies regulatory elements controlling gene expression and modulation in bone disorders. The regulatory elements and reagents described in the present invention facilitate the study and development of products and methods to increase the mineral content of bone, which can consequently be utilized to treat a wide variety of bone related conditions, including, osteopenia, osteoporosis, fractures and other disorders in which low bone mineral density are the main cause of the disease. Furthermore, the present invention provides regulatory elements and reagents useful for bone pattering and growth, limb development, and the formation of individual bones, particularly how very similar bones establish their identity such as fingers and toes, or how bone outgrowth proceeds from shoulder to finger tips.

Sclerosing bone dysplasias are rare genetic disorders in which excessive bone formation occurs due to defects in bone remodeling. Identifying the responsible genes, their regulation and mechanisms of action will provide useful insights into bone physiology and potentially benefit the treatment of these disorders, as well as facilitate the development of therapies for replenishing bone loss in osteoporosis and other related disorders.

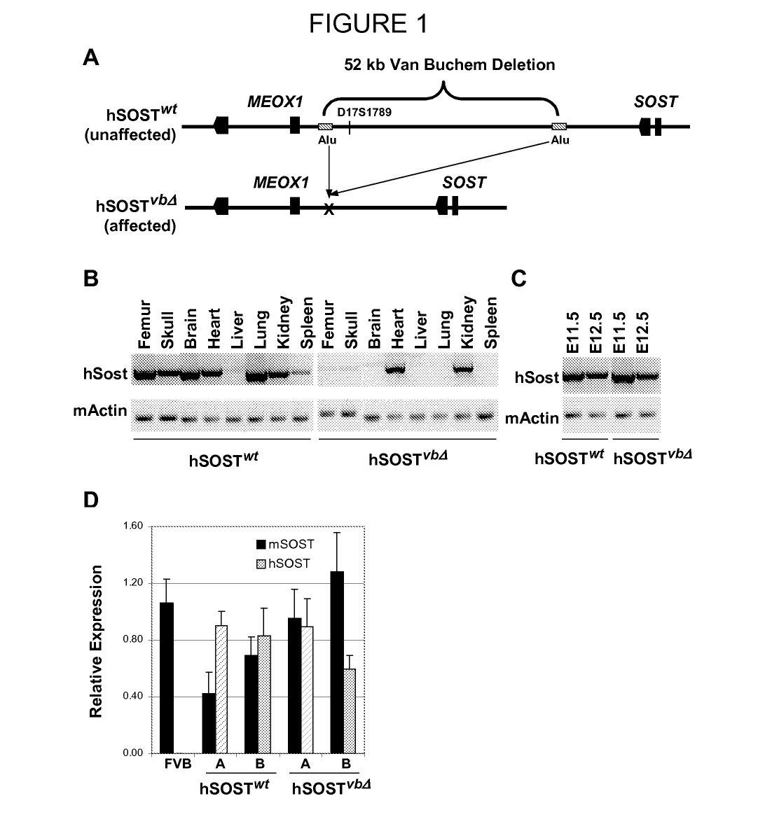

An exciting development has been the recent discovery of a negative regulator of bone formation, sclerostin (SOST) (Balemans, W., e et al., 2001. Increased bone density in sclerosteosis is due to the deficiency of a novel secreted protein (SOST). Hum Mol Genet. 10: 537-543;), whose expression is affected in both sclerosteosis and Van Buchem disease. Whereas sclerosteosis patients carry homozygous null SOST mutations, VB patients lack any SOST coding mutations (Van Bezooijen, R. L., et al., 2004. Sclerostin Is an Osteocyte-expressed Negative Regulator of Bone Formation, But Not a Classical BMP Antagonist. J Exp Med 199: 805-814; Winkler, D. G., et al., 2003. Osteocyte control of bone formation via sclerostin, a novel BMP antagonist. Embo J22: 6267-6276). They do however, carry a homozygous 52 kb noncoding deletion (vbΔ) ˜35 kb downstream of the SOST transcript and ˜10 kb upstream of the downstream gene, MEOX1, on human chromosome 17p21 (FIG. 1A). The shared clinical similarities between VB and sclerosteosis along with their strong genetic linkage to the SOST locus on chromosome 17q12 suggests that they are allelic, and that the deletion in VB patients removes an enhancer element essential for directing the expression of human SOST in the adult skeleton.

To gain insight into the mechanism by which this newly discovered gene impacts bone patterning and remodeling in Van Buchem disease, as well as to characterize the transcriptional regulation of sclerostin, in the Examples we have characterized human BAC SOST transgenic mice carrying either a normal (SOST^{wt}) or an allele with the VB associated deletion (SOST^{wbΔ}). Only the SOST^{wt} allele faithfully expresses human SOST in the adult bone and impacts bone metabolism, consistent with the model that the VB noncoding deletion removes a SOST-specific regulatory element. By exploiting cross-species sequence comparisons with in vitro and in vivo enhancer assays we have identified several putative regulatory elements, in particular one enhancer element that drives human SOST expression in the adult mouse skeleton, and discovered a novel function for sclerostin during limb development, demonstrating that this very important skeletal dysplasia, Van Buchem disease, is caused by the removal of a bone-specific distant enhancer element and is allelic to sclerosteosis.

Our study also provides strong support for the utilization of comparative sequence analysis to dramatically filter through nonfunctional regions in the human genome and enhance the discovery of noncoding disease-causing mutations both in discrete enhancer elements or in large noncoding deletions. This study represents a clear and unambiguous case where altering noncoding genomic content deleteriously impacts gene expression, demonstrating that mutations in distant regulatory elements are able to cause congenital abnormalities analogous to coding mutations.

### DEFINITIONS

A “cell of bone lineage” refers to any cell that found in bone or can develop into a cell found in bone. Such cells include, e.g. mesenchymal cells, osteoblasts, osteoclasts, and osteocytes.

“Sclerostin” and “SOST” refer to a bone morphogenic protein (BMP) antagonist that is a negative regulator of bone formation. Human SOST is expressed in primary human osteoblasts, osteocytes, mesenchymal cells differentiated in culture to osteoblasts, and hypertrophic chondrocytes in cartilage tissue.

“Regulatory element” refers to a nucleotide sequence that modulates the expression of an upstream or downstream nucleic acid. Regulatory elements include, e.g., enhancers and repressors.

“ECR” refers to an evolutionarily conserved region (i.e., sequence) within the van Buchem disease-associated noncoding deletion region that regulate (i.e., enhance or repress) expression of SOST. ECR sequences are set forth in SEQ ID NOS: 1-15 and 17-59.

The terms “substantial identity” or “substantial similarity” refer to a nucleic acid or fragment thereof which is “substantially identical” (or “substantially similar”) to another if, when optimally aligned (with appropriate nucleotide insertions or deletions) with the other nucleic acid (or its complementary strand), using BLASTN there is nucleotide sequence identity (“% ID”) in at least about 60%, 65%, 70%, 75%, 80%, 85%, 90%, 91%, 92%, 93%, 94%, 95%, 96%, 97%, 98%, or 99% or more of the nucleotide bases. In a preferred embodiment, to determine homology between two different nucleic acids, the percent homology is to be determined using the BLASTN program “BLAST 2 sequences”. This program is available for public use from the National Center for Biotechnology Information (NCBI) over the Internet (URL:<http://www.ncbi.nlm.nih.gov/gorf/b12.html>) (Altschul et al., 1997). In a preferred embodiment, the parameters to be used are whatever combination of the following yields the highest calculated percent homology (as calculated below) with the default parameters shown in parentheses:

Program—blastn

### Matrix—0 BLOSUM62

Reward for a match—0 or 1 (1)
Penalty for a mismatch—−0, −−1, −2 or −3 (−2)
Open gap penalty—0, 1, 2, 3, 4 or 5 (5)
Extension gap penalty—0 or 1 (1)
Gap x_dropoff—0 or 50 (50)

### Expect—10

The terms “substantial homology” or “substantial identity”, when referring to polypeptides, indicate that the polypeptide or protein in question exhibits at least about 30% identity using BLASTP with an entire naturally-occurring protein or a portion thereof, usually at least about 60%, 65%, 70%, 75%, 80%, 85%, 90%, 91%, 92%, 93%, 94%, 95%, 96%, 97%, 98%, or 99% or more identity over common lengths.

Homology, for polypeptides, is typically measured using sequence analysis software. See, e.g., the Sequence Analysis Software Package of the Genetics Computer Group, University of Wisconsin Biotechnology Center, 910 University Avenue, Madison, Wis. 53705. Protein analysis software matches similar sequences using measures of homology assigned to various substitutions, deletions and other modifications. Conservative substitutions typically include substitutions within the following groups: (a) glycine, alanine; (b) valine, isoleucine, leucine; (c) aspartic acid, glutamic acid; (d) asparagine, glutamine; (e) serine, threonine; (f) lysine, arginine; and (g) phenylalanine, tyrosine.

The term “polynucleotide” refers to a chain of nucleotides without regard to length of the chain.

The term “polypeptide” refers to a polymer of amino acids without regard to the length of the polymer; thus, peptides, oligopeptides, and proteins are included in this term.

SOST Gene Regulatory Elements

One embodiment of the invention provides nucleotide sequences for SOST gene regulatory elements. Sequences for ERC1, ERC2, ERC3, ERC4, ERC5, ERC6, ERC7, ERC8, ERC9, ERC10, ERCA, ERCB, ERCC, ERCD, and ERCE are set forth in SEQ ID NOS: 1-15. The regulatory elements described herein can be used to create constructs that delete all or specific SOST regulatory elements, e.g., to generate recombinant cell lines or transgenic animals.

In order to study the physiological and phenotypic consequences of a lack of the disclosed SOST enhancer elements, both at the cellular level and at the organism level, the preferred embodiment also encompasses DNA constructs and recombinant vectors enabling conditional expression of a specific allele or haplotypes of the SOST genomic sequence or a SOST cDNA as described in SEQ ID NO: 16 in a transgenic, knock-out, or knock-in non-human animal. The embodiment also encompasses DNA constructs to generate animals having multiple copies of SOST regulatory elements (individuals, or combinations, one or more copies of each enhancer), polymorphic variants of individual copies (base pair changes or small deletions) to modulate expression of the Sost protein expressed (or reporter gene such as beta-galactosidase [LacZ] or green fluorescent proteins [GFP]) and animals having decreased or no Sost protein expressed due to lack of the disclosed SOST regulatory elements (“knock-out animals”).

The targeting construct can be built by various methods known in the art including but not limited to, PCR primers for integration by homologous recombination, using a repressor/marker promoter construct, Cre-LoxP system, and antisense constructs. The method preferred is using PCR products and primers to build the targeting construct. To build such a construct to make knockout non-human animals and cells, one would need the homology “arms” that flank each side of the sequence to be deleted or disrupted, and a selectable marker inserted between the arms to select for the marker function. The sequence to be deleted can be the whole Van Buchem region described in Example 1, parts of the VB deletion region, the SOST gene or parts of SOST, or any of the SOST regulatory elements, single or multiple exons, introns, intervening genomic sequences up to the nearest neighboring gene on each side, short peptide sequences and even single base pair deletions, insertions, or substitutions. After delivery of the construct into embryonic stem cells (for knockouts) or blastocyts/one cell stage embryos (for transgenics), selection for the marker permits gene deletion, Or for instance, SOST regulatory element function can be disrupted by the insertion of a selectable marker, by deletion, or by a mutation (base pair replacement).

Transgenic Models for SOST Gene Regulatory Elements

To make transgenic non-human animals, designing the construct may include as much flanking sequence of the target sequence to be deleted as to include all the enhancer and regulatory elements that may be found in the flanking genomic DNA. One needs to consider the neighboring genes and whether or not they should be over-expressed as well. See Thomas, K. R. and Capecchi, M. R., Site-directed mutagenesis by gene targeting in mouse embryo-derived stem cells. Cell 51:503, 1987.

Thus in a specific embodiment, SEQ ID NOS: 1-15, or a substantially similar sequence of the SOST regulatory elements of the present invention, can be used to create constructs that delete all or specific SOST regulatory elements. In a preferred embodiment, the targeting construct to delete the SOST regulatory elements can be built using PCR products and primers such as SEQ ID NOS: 81-84. For example, ECR5 knockout mice can be generated by deleting the ECR5 sequence in the genome using SEQ ID NOS: 71-72.

In order to effect expression of the polynucleotides and polynucleotide constructs of the preferred embodiment, these constructs must be delivered to the host cell, where once it has been delivered to the cell, it may be stably integrated into the genome of the host cell and effectuate cellular expression. This delivery can be accomplished in vitro, for laboratory procedures for transforming cell lines, or in vivo or ex vivo, for the creation of therapies or treatments of diseases. Mechanisms of delivery include, but are not limited to, viral infection (where the expression construct is encapsulated in an infection viral particle), other non-viral methods known in the art such as, calcium phosphate precipitation, DEAE-dextran, electroporation, direct micro-injection, DNA-loaded liposomes, and receptor-mediated transfection of the expression construct. In a preferred embodiment, the delivery of the construct is by micro-injection into the appropriate host cell or by intravenous injection in the organism.

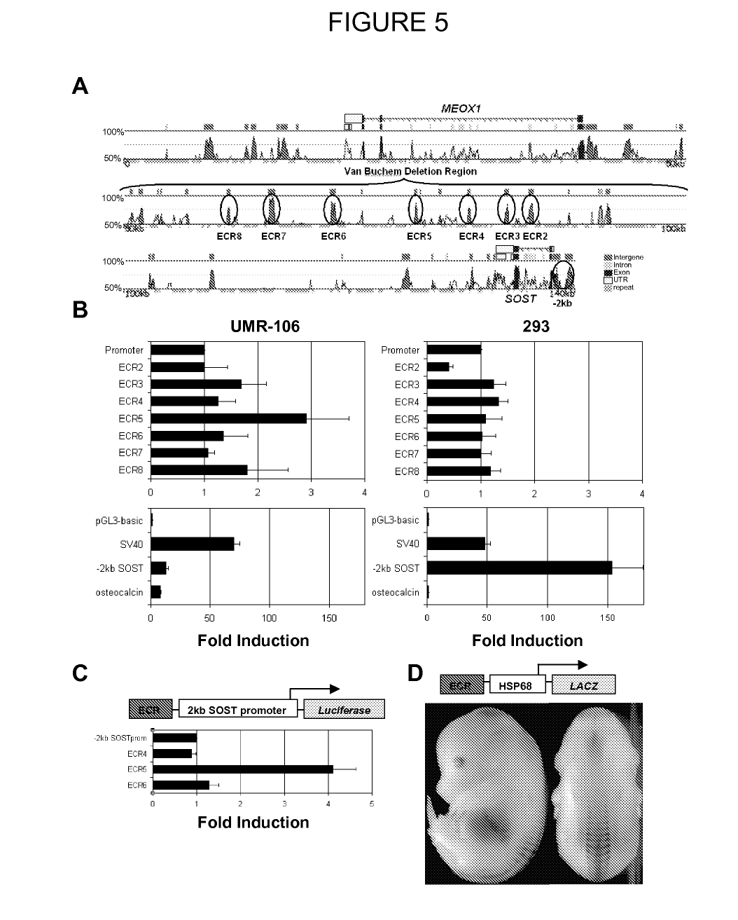

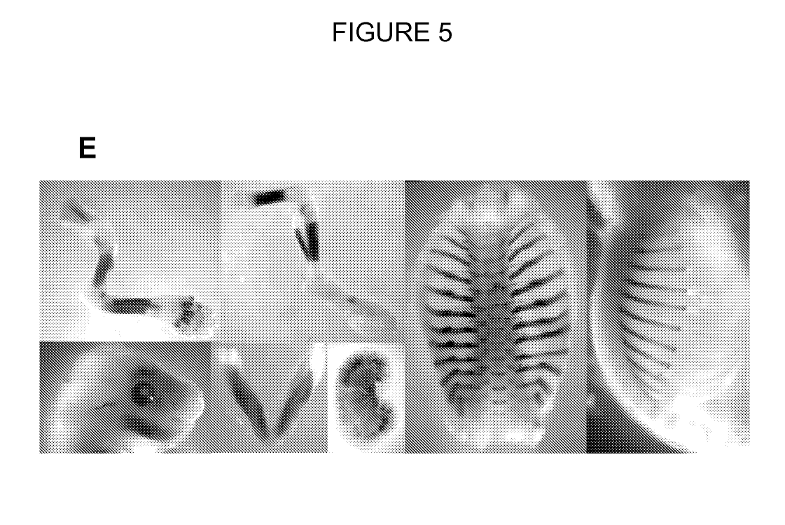

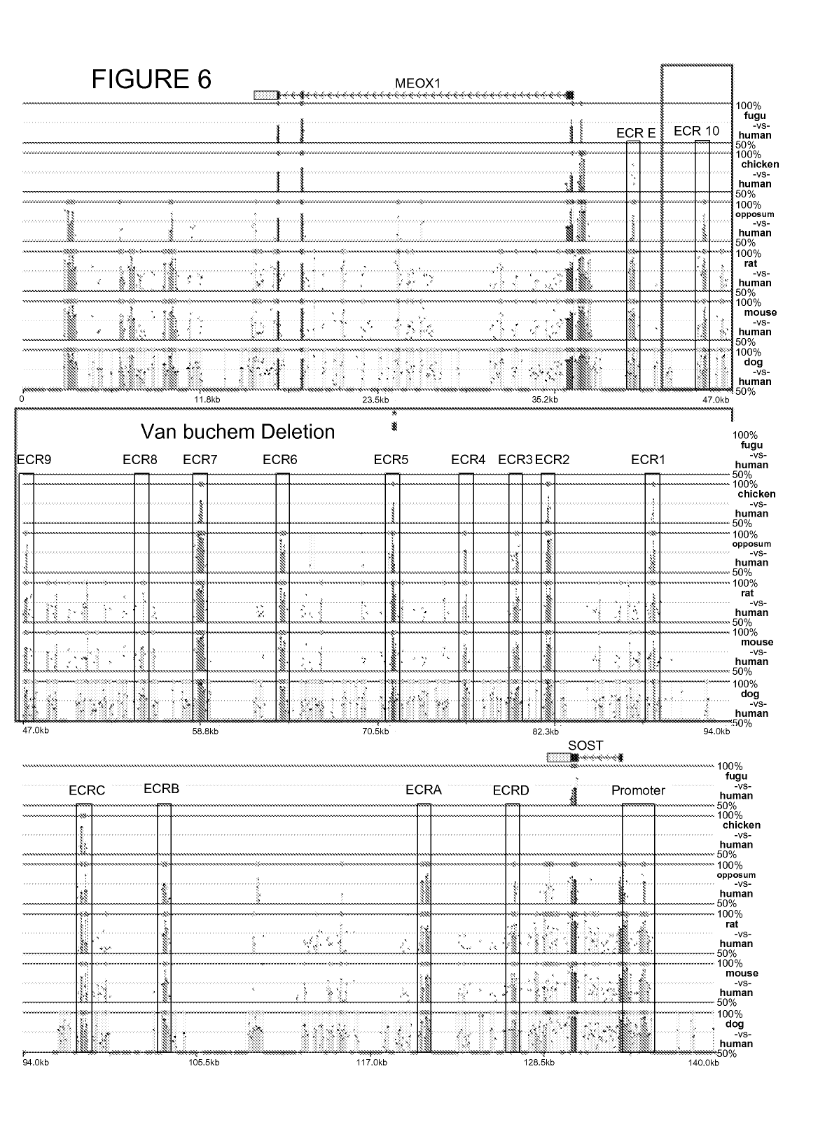

In the Examples, to test ECR5's ability to drive expression in the skeletal structures of the mouse embryo, an ECR-hsp68-LacZ construct was expressed in transgenic mice (FIG. 5D) (Nobrega, M. A., I. Ovcharenko, V. Afzal, and E. M. Rubin. 2003. Scanning human gene deserts for long-range enhancers. Science 302: 413). Transient transgenic animals were created using standard techniques (Mortlock, D. P., C. Guenther, and D. M. Kingsley. 2003. A general approach for identifying distant regulatory elements applied to the Gdf6 gene. Genome Res 13: 2069-2081) and F0 pups were stained for β-galactosidase expression at E14.5 (Nobrega et al. 2003). Transgenic embryos expressed LacZ in cartilage of the ribs, vertebrae and skull plates (FIG. 5D). LacZ expression in the adult transgenics was counterstained with bone and cartilage markers, and transgene expression was consistently observed in the skeletal structures. These data confirm that the 250 basepair (bp) ECR5 contained within the 52 kb VB region is indeed a bone specific enhancer in vivo.

Therefore, the invention provides homozygous knockout non-human animals that are lacking any or all of the SOST regulatory elements described herein and therefore down regulate expression and production of SOST protein. These animals will have decreased SOST levels and thereby modulating bone density levels. This invention also includes recombinant vectors and DNA targeting constructs, such as the one used by the inventors to delete mouse VB deletion and was built using PCR products and primers made from SEQ ID NOS: 81-84.

The invention further provides non-human animals that over-express any of the human SOST regulatory elements described herein. The over-expression of human SOST under the control of its own proximal promoter elements in concert with the downstream VB region negatively modulates adult bone mass. In development, the over-expression of any of these regulatory elements to increase SOST levels in animals missing the VB region can be used to affect bone, limb and digital development.

This invention also provides non-human animals for further animal studies by pharmaceutical companies to study human or mouse (or derived from other species) SOST enhancer and regulatory elements. Animal studies that explore the regulation and expression of human or mouse SOST, its interaction with other related proteins, production of antibodies for mutant and wild-type SOST regulatory elements or antibodies that specifically bind to proteins that specifically interact with SOST regulatory elements, and further in vivo study of SOST and its enhancer elements. For example, wild-type mice or rats may be exposed to various test ECR5 inhibitors to determine the SOST lowering effect of the test substance and the consequent ability to stimulate bone formation and growth (including, e.g. osteoclast/osteoblast/osteocyte differentiation, function, and proliferation). The invention further provides non-human animals useful for studying ostepenia and osteoporosis by reducing Sost expression through the inhibition of the enhancer element ERC5 in (e.g., in ovarectomized (OVX) rats or mice (or similar osteopenic animals) and monitoring anabolic bone effects, and recovery from bone loss.

### I. Diagnostic Applications Using SOST Regulatory Elements

The present embodiment enables diagnostic and therapeutic compositions, methods and applications based on the finding that modulation of SOST can be carried out by the regulatory elements described herein, ERC1, ERC2, ERC3, ERC4, ERC5, ERC6, ERC7, ERC8, ERC9, ERC10, ERCA, ERCB, ERCC, ERCD, ERCE, and combinations thereof. In some embodiments, the regulatory element comprises a sequence selected from SEQ ID NOS: 1, 2, 3, 4, 5, 6, 7, 8, 9, 10, 11, 12, 13, 14, and 15. In some embodiments, the regulatory element is an enhancer (e.g., ERC5). In some embodiments, the ERC5 comprises the sequence set forth in SEQ ID NO: 5. Therefore, the present invention also provides methods of modulating bone mineral density in a subject by providing a composition that inhibits SOST expression via the one of the above regulatory elements located within the region deleted in VB patients. In a preferred embodiment, the regulatory element is the ECR5 enhancer (or other similar sequences located within the region deleted in VB patients), and administering a therapeutically effective amount of that composition to the subject to modulate ECR5 activity, and thereby modulate SOST gene expression to regulate bone growth and development, and to stimulate anabolic bone formation.

1. Genotyping and Haplotyping

The present embodiment enables genetic testing for polymorphisms in SOST regulatory elements, deletion of discrete SOST-specific regulatory elements and the VB deletion and its correlation to abnormal digit development in people having deletions deviating from the normal or “wild type” genotype. Further, a combination test with SOST or other conserved sequences described herein is suggested. Genetic testing may be carried out on a patient's DNA or RNA or protein, provided that antibodies are capable of distinguishing different levels of sclerostin.

As demonstrated in the examples below, noncoding regions in the VB deletion control Sclerostin expression levels and modulate BMD in mice, therefore an important question is whether variation in BMD in the general population could also be directly impacted by sequence variants in key noncoding regions of the VB deletion. A recent new study investigated the association between common polymorphisms in the SOST gene region with BMD in elderly whites (Uitterlinden, A. G., P. P. Arp, B. W. Paeper, P. Charmley, S. Proll, F. Rivadeneira, Y. Fang, J. B. van Meurs, T. B. Britschgi, J. A. Latham, R. C. Schatzman, H. A. Pols, and M. E. Brunkow. 2004. Polymorphisms in the sclerosteosis/van Buchem disease gene (SOST) region are associated with bone-mineral density in elderly whites. Am J Hum Genet. 75: 1032-1045). From a set of 8 polymorphisms, one 3-bp deletion (SRP3) from the SOST promoter region was associated with decreased BMD in women, and a polymorphic variant (SRP9) from the VB deletion region was associated with increased BMD in men. Whereas this SRP9 does not map on any human-mouse conserved region in the VB deletion, an important question for future studies is whether this SNP is in linkage disequilibrium with ECR5 or other conserved sequences described herein and if additional functional SNPs could be identified in this or other SOST-specific regulatory elements.

The genetic factors that contribute to susceptibility to bone loss are extremely heterogeneous, therefore murine models that affect bone development and growth can provide invaluable insights into the molecular mechanisms of progressive bone loss in humans. Human genetic diseases of the skeleton such as sclerosteosis and Van Buchem disease provide a starting point for understanding the modulation of anabolic bone formation, and ultimately have the potential to identify key molecular components that can be used as new therapeutic agents to treat individuals suffering from bone loss disorders. The study described in the Examples also provides strong support for the utilization of comparative sequence analysis to dramatically filter through nonfunctional regions in the human genome and enhance the discovery of noncoding disease-causing mutations in regulatory elements including in discrete enhancer elements or in large noncoding deletions. This study represents a clear and unambiguous case where altering noncoding genomic content deleteriously impacts gene expression, demonstrating that mutations in distant regulatory elements are able to cause congenital abnormalities analogous to coding mutations.

Thus the present invention would provide a test for whether an individual, such as a fetus, has an ECR5SNP (or small basepair composition change such as small deletion or insertion) or additional functional SNPs identified in the described or other SOST-specific regulatory elements. It is also contemplated that such a test would also be used in conjunction or include the eight SNPs found and described in Uitterlinden et al. 2004. None of the SNPs described in Uitterlinden fall in conserved SOST regulatory element sequences of the present invention.

Any method known in the art can be used to identify a nucleotide polymorphism, small deletion or insertion present at one of the disclosed SOST regulatory elements. Detection and identification of SNPs and haplotypes in the disclosed SOST regulatory elements in the present invention can be accomplished by one of ordinary skill in the art. Any number of techniques to detect the haplotype of an individual by genotyping the individual at certain polymorphic sites can be used, including, but not limited to, the methods set forth herein.

The nucleotide can be determined by sequencing analysis after DNA samples are subjected to PCR amplification. Preferably, the amplified DNA is subjected to automated dideoxy terminator sequencing reactions using a dye-primer cycle sequencing protocol. The sequencing reactions are then sequenced using any number of commercially available sequencing machines such as the ABI 377 or 3700 Sequence Analyzer (Applied Biosystems, Foster City, Calif.).

Techniques and methods of synthesizing and amplifying polynucleotides by ligation of multiple oligomers (LMO) onto a template-bound primer are also described by Akhavan-Tafti in U.S. Pat. Nos. 5,998,175; 6,001,614; 6,013,456; and 6,020,138, which are hereby incorporated by reference in their entirety. Short polynucleotides, 5 to 10 bases long, can be supplied as a library of oligonucleotides and are simultaneously ligated, using a suitable ligase enzyme, to a template-bound primer in a contiguous manner to produce a complementary strand of template polynucleotide. If the sequence to be synthesized is known, a set containing the minimum number of oligomers can be used and are then ligated by DNA Ligase in the correct order starting from the primer, uni- or bi-directionally, to produce the complementary strand of a single-stranded template sequence.

A preferred method is to use sequence detection/amplification assays such as the INVADER assays which are commercially available from Third Wave Technologies (Madison, Wis.) to genotype samples. Such systems rely on an enzyme-substrate reaction to amplify signal generated when a perfect match with an (rare) allele of a SOST regulatory element is detected. See Dahlberg, J. et al., U.S. Pat. Nos. 5,846,717 and 5,888,780, which are hereby incorporated by reference in their entirety.

A third preferred method is using methods that have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis) See U.S. Pat. Nos. 5,547,835; 6,221,601; 6,194,144 which are hereby incorporated by reference in their entirety. Other methods of SNP analysis are performed by companies such as Sequenom (San Diego, Calif.), which can genotype many samples very quickly and with great accuracy non-sequencing methods such as MALDI-TOF, miniaturized chip-based array formats and mass spectrometry.

Other genotyping methods suited for detection of SNPs include, but are in no way limited to, LCR (ligase chain reaction), Gap LCR (GLCR), using allele-specific primers, mismatch detection assays, microsequencing assays, and hybridization assay methods.

2. Methods of Genetic Analysis and Association Studies

In general, the SNPs of this invention find use in any method known in the art to demonstrate a statistically significant correlation between a genotype and phenotype, and between a haplotype and an enotype. Preferably, the SNPs are used in studies to determine their correlation to bone and bone density disorders. More preferably, the SNPs are used in studies to determine whether they are causative mutations of bone disorders.

The described polymorphisms can be used to separate individuals based on any phenotypic trait. For instance, patients can be treated with standard and current bone therapies and their bone density levels can be determined. Individuals can then be separated based on their ECR5 genotype/haplotype and their average bone density level determined. This will enable a physician to address if ECR5 polymorphisms influence how responsive an individual will be to a specific bone therapy.

A similar strategy could be used for any drug therapy. As another example, a certain diseased group of individuals could be separated based on their SOST or ECR5 genotype/haplotype, and all the average phenotypes from these groups can be examined for differences. For example, if a particular phenotype display shows a difference, the phenotype would be identified as a phenotype that ECR5 may influence. For instance, a group suffering from osteoporosis could be separated based on their ECR5 or ECR5/SOST genotype. Numerous phenotypes in these subgroups can be averaged and compared according to bone density levels. If there is a difference in bone density levels, this would support the proposal that ECR5 influences bone density levels in osteoporosis. Another example would be to look at specific bone diseases to see if there is an increased frequency of the minor haplotypes in the diseased group compared to controls. If there is a difference in frequency, then ECR5 likely contributes to this disease.

Criteria or methods for selecting individuals for treatments, drug trials and any of the studies described herein include, but are not limited to, such criteria for eligibility as: willingness to participate in program, no medication use likely to interfere with total body bone mineral content or bone metabolism, percentage of ideal body weights according to such tables and indices available such as Metropolitan Life Insurance Company Tables (1985), certain body mass index, free of chronic disease, nonsmoker, using hormone replacement therapy, related or unrelated to other subjects in the study, family and other relatives living and willing to submit to studies, belonging to certain age and/or ethnicity groups, possessing defined levels of bone density, strength and frequency of exercise and activity, adherence to diet and/or exercise protocol and requirements, any past injuries or bones broken, total body composition and biochemical indices of bone turnover over a defined period, and any other measurable genotypic or phenotypic trait. In addition to meeting these criteria, analysis of the bone density of the subjects should be done to develop complete profiles of each subject.

For more examples of preferred subject criteria and methods of measuring changes in bone mineral density, total body composition and biochemical indices of bone turnover over a defined period, and the normal ranges of proteins such as serum osteocalcin, calcitonin, bone-specific alkaline phosphatase, urinary NTX/creatinine excretion, macrophage colony stimulating factor (M-CSF) and receptor activator of NFeB ligand (RANKL), serum 25-hydroxyvitamin D, and serum parathyroid hormone levels, and methods for conducting studies and clinical trials as herein described, see Kaskani E, et al., Effect of intermittent administration of 200 IU intranasal salmon calcitonin and low doses of 1 alpha(OH) vitamin D(3) on bone mineral density of the lumbar spine and hip region and biochemical bone markers in women with postmenopausal osteoporosis: a pilot study; Gordon C M, et al., Effects of oral dehydroepiandrosterone on bone density in young women with anorexia nervosa: a randomized trial, Clin Rheumatol. 2005 Jan. 13; [Epub ahead of print], and J Clin Endocrinol Metab. 2002 November; 87(11):4935-41; and Felsenberg D, Boonen S, The bone quality framework: determinants of bone strength and their interrelationships, and implications for osteoporosis management, Clin Ther. 2005 January; 27(1): 1-11, which are hereby incorporated by reference in their entirety.

A preferred embodiment permits genetic analysis studies between disclosed SNPs, the SOST regulatory elements ERC1-10, and ERCA-E and any phenotype. In general, the regulatory elements of the present invention find use in any method known in the art to demonstrate a statistically significant correlation between a genotype and phenotype. The genetic analysis using the SNPs and regulatory elements that may be conducted include but are not limited to linkage analysis, population association studies, allele frequencies, haplotype frequencies, and linkage disequilibrium.

Linkage analysis is based upon establishing a correlation between the transmission of genetic markers and that of a specific trait throughout generation within a family. Thus, the aim of linkage analysis is to detect marker loci that show co-segregation with a trait of interest. Linkage analysis correlating SOST SNPs and regulatory elements and the trait of high or low bone density levels within families or people/ethnic groups are an aim of this invention. Further linkage analysis is also contemplated for studies of other people and ethnic groups, and further regional studies including groups in other countries. Linkage analysis can be performed according to parametric or non-parametric methods.

Frequency of alleles and haplotypes in a population is also another genetic analysis study contemplated by the invention. Using the genotyping and haplotyping methods described herein and known in the art, one skilled in the art can determine the frequency of SOST and/or any SOST regulatory elements and SNPs found in a given population. While several methods of estimating allele frequency are possible, genotyping individual samples is preferred over genotyping pooled samples due to higher sensitivity, reproducibility and accuracy. Furthermore, many genomic and large-scale sequencing centers enable rapid genotyping and haplotyping by sequencing methods and thereby provide rapid data production.

Association studies between SOST and SOST regulatory enhancers and/or SNPs (or other base pair composition change such as small deletion or insertion) and any phenotype can also be performed on a random sample of people, anywhere from a few hundred to tens of thousands. After collecting various parameters for each individual participating in the study, such as height, weight, bone mass and density levels, medical history, etc., the sample group can be separated according to various genotypes. Any repeated differences in the parameters in individuals that are observed are likely traits that are associated with one of the SOST or SOST regulatory element genotypes. The Examples show that there are differences in bone mass and density levels that are associated with ECR5 enhancer genotype, however, there are likely other associations that can be subject to study. Other parameters to observe include, but are not limited to, presence of bone disease risks, other hormone, mineral and protein levels, instances of other diseases or conditions, age and gender.

Studies correlating the genotype/haplotype with methods and treatments of bone diseases and disorders are also contemplated. Segregation of individuals in the study according to their response (e.g. increase of bone density levels) to various drug therapies and combinations with inhibitors of any of the disclosed regulatory elements and then according to allele frequency. The result of stratification of population studies would enable doctors and medical care providers to prescribe therapy with greater accuracy, and with greater success rates. Thus, therapy prescribed would be “tailor-made” for individuals based upon their genotypes.

Statistical methods and computer programs useful for linkage analysis, genetic analysis and association studies are well-known to those skilled in the art. Any statistical tool useful to test for statistically significant associations between genotypes, haplotypes and phenotypes, comparisons and correlations between a biological marker and any physical trait, and frequency comparisons may be used.

Statistical analyses can be carried out using the SAS computer program (SAS, Cary, N.C.) and similar programs. Bone mass and density levels can be compared among different genotype groups using Wilcoxon's test and the like. Allele frequencies should be compared using such tests as Fisher's exact test. To determine pairwise linkage disequilibrium (LD) between SNPs, haplotype frequencies, estimations can be done using the Expectation-Maximization (EM) algorithm implemented in the computer program ARLEQUIN v. 2.0 ((Excoffier and Slatkin, Mol. Biol. Evol. 1995, 12 (5):921-927), and downloadable from URL:<http://lgb.unige.ch/arlequin/>), an exploratory population genetics software environment.

Pair-wise measure of linkage disequilibrium (|D′|) can be calculated for all combinations of frequencies as described by R. C. Lewontin, Genetics 120, 849-52 (1988). A |D′| value of 1 indicates complete linkage disequilibrium between two markers.

Examples of useful statistical methods and techniques include Analysis of Variance (ANOVA), Fischer's test for pair-wise comparison and Wilcox's test, generally carried out using programs such as SPSS (Chicago, Ill.), STATVIEW and SAS (both available from SAS, Cary, N.C.).

### II. Therapeutic Applications Using SOST Regulatory Elements

The present invention provides for various therapeutic applications using the described SOST regulatory elements and their ability to modulate SOST expression and bone mass density. Using the disclosed sequence of the SOST regulatory elements, inhibitors (or down-regulators) of these regulatory elements or proteins that physically interact with the regulatory elements can be made as described herein and as is known in the art. Such inhibitors include, but are not limited to such materials as antibodies, olignonucleotides, aptamers, and viral vectors that deliver, produce or express these sequences and small molecule inhibitors that inhibit the function of the SOST regulatory elements to modulate SOST expression (i.e., either upregulate or downregulate SOST). Alternatively regulatory proteins that normally bind to ECR5 or any other regulatory element described herein to stimulate SOST expression can be inhibited by physically preventing them to associate with the regulatory sequence, or by rendering their activity inert by preventing post-translational modifications if, e.g., protein covalent modifications are required for normal protein activity such as phosphorylation, sumoylation, and the like. This inhibition can be mediated by, but it is not limited to, materials such as antibodies, small inhibitory peptides or chemical compounds, antisense oligonucleotides, si/shRNA olignonucleotides, aptamers, and viral vectors that deliver, produce or express these sequences and small molecule inhibitors whose overall effect is to prevent the interaction of a regulatory protein with a SOST-specific regulatory element.

In one embodiment, the therapeutic inhibitors of the present invention can be used to treat or prevent a variety of disorders associated with any bone loss disease such as osteporosis or osteopenia. Osteoporosis is a skeletal disease characterized by bone loss and deterioration of bone tissue, with a consequent increase in bone fragility and susceptibility to fracture. It is often observed in the elderly and especially in post-menopausal women. Clinical studies have noted that the loss of estrogen in post-menopausal women contributed to their loss of bone mass and hormone replacement therapy (HRT) has been prescribed to counter the effects of osteoporosis in these women. See Gambacciani M, Vacca F. in Minerva Med. 2004 December; 95(6):507-20. However, prolonged exposure to HRT also increased risks associated with it, including increased risks of developing breast cancer and endometrial cancer in post-menopausal women over age 50 (see Ewertz M, et al., Br J. Cancer. 2005 Apr. 11; 92(7): 1293-7) and increase in incidence of atherosclerotic cardiovascular diseases. See Hoshino S, Ouchi Y, Clin Calcium. 2004 November; 14(11):87-98. Therefore, in the case of post-menopausal women suffering from bone loss, the present invention would promote the treatment of bone density loss without the drawbacks of hormone replacement therapy because the present invention would inhibit or block in vivo bone specific regulatory elements, and are not associated with other hormone pathways.

Subjects suffering from bone diseases including, osteoporosis, osteoporosis-induced by glucocorticoid therapy or anorexia nervosa or asthma, osteosarcoma, osteopenia and Crohn's disease, as well as patients suffering from renal diseases and arthritis may further benefit from the therapeutics described herein.

In one embodiment, targeting regulatory elements could also have an application for treating sclerosteosis and VB patients. In general, the patients appear normal until about age 5. Genotyping methods can be used to determine whether patients have the VB deletion or mutations in enhancer within the VB region. After diagnosis, stimulators of SOST activity via the promoter, regulatory elements or downstream effector proteins can be used to upregulate or stimulate SOST activity in these patients. If SOST can be upregulated, the patients may not develop some of the severe long-term side effects associated with increased bone growth such as nerve pinching, and possible hearing loss and blindness.

In another embodiment, the SOST regulatory element inhibitory polynucleotides and polypeptides can be isolated, recombinant or synthesized, so long as the polynucleotides and polypeptides inhibit ECR2-8 functionality and SOST expression.

1. Antibodies to SOST Regulatory Elements ECR1-10, ECRA-E and their Variants

Antibodies including both polyclonal and monoclonal antibodies, and drugs that modulate the production and activity of SOST, and may possess certain therapeutic applications. Such antibodies may, for example, be utilized for the purpose of inhibiting ECR5 function or any combination of the ECR1-ECRE (SEQ ID NOS: 1-15) to modulate the activity or production of SOST, or inhibit regulatory proteins that normally associate with SOST-specific regulatory elements and function to stimulate the production and activity of SOST.

For example, wild type ECR1-10 and ERCA-E, their variants, or peptides interacting with wild-type SOST regulatory elements may be used to produce both polyclonal and monoclonal antibodies in a variety of cellular media, by known techniques such as the hybridoma technique utilizing, for example, fused mouse spleen lymphocytes and myeloma cells. Likewise small molecules that mimic or agonize the activity(ies) of SOST-regulatory elements or proteins that normally bind to and modulate the function of SOST-regulatory elements may be discovered or synthesized, and may be used in diagnostic and/or therapeutic protocols.

The general methodology for making monoclonal antibodies by hybridomas is well known. Immortal, antibody-producing cell lines can be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890, all of which are hereby incorporated by reference.

Panels of monoclonal antibodies produced that specifically bind to peptides that interact with ECR1-10 and ERCA-E or that specifically bind to the regulatory elements themselves can be screened for various properties; i.e., isotype, epitope, affinity, etc. Of particular interest are monoclonal antibodies that specifically bind and identify the alleles of ECR5 and ECR 1-10 and ERCA-E and can distinguish between the rare and the normal alleles of these regulatory elements. In one preferred embodiment, a monoclonal antibody can be generated that specifically binds to ECR5, and any specific positions in ECR5 which correspond or result from single nucleotide polymorphisms (SNP) and sequence variants. Such monoclonals can be readily identified in, for example, gel-shift assays.

A preferred method of generating allele-specific antibodies to ECR5, or any of the regulatory elements ECR1-10 and ERCA-E, is by first synthesizing peptide fragments. Peptide fragments to any regulatory element should cover any SNPs or sequence variants along with the adjacent amino acid sequence. Subsequent antibodies should be screened for their ability to distinguish the two variants. Since synthesized peptides are not always immunogenic on their own, the ECR5, ECR1-10 or ECRA-E peptides should be conjugated to a carrier protein before use. An appropriate carrier proteins includes but is not limited to Keyhole limpet hemacyanin (KLH). The conjugated peptides should then be mixed with adjuvant and injected into a mammal, preferably a rabbit through intradermal injection, to elicit an immunogenic response. Samples of serum can be collected and tested by ELISA assay to determine the titer of the antibodies and then harvested as is known in the art.

Polyclonal ECR1-10 and ERCA-E allele-specific antibodies can be purified by passing the harvested antibodies through an affinity column. Monoclonal antibodies are preferred over polyclonal antibodies and can be generated according to standard methods known in the art of creating an immortal cell line which expresses the antibody.

Additionally, spleen cells can be harvested from the immunized animal (typically rat or mouse) and fused to myeloma cells to produce a bank of monoclonal antibody-secreting hybridoma cells. The bank of hybridomas can be screened for clones that secrete immunoglobulins that bind the protein of interest specifically, i.e., with an affinity of at least 1×10⁷ M⁻¹. Animals other than mice and rats may be used to raise antibodies; for example, goats, rabbits, sheep, and chickens may also be employed to raise antibodies reactive with any of the ECR2-8 regulatory elements. Transgenic mice having the capacity to produce substantially human antibodies also may be immunized and used for a source of antiserum and/or for making monoclonal antibody secreting hybridomas using methods accepted and known in the art.

Bacteriophage antibody display libraries may also be screened for phage able to bind peptides and proteins specifically. Combinatorial libraries of antibodies have been generated in bacteriophage lambda expression systems and may be screened as bacteriophage plaques or as colonies of lysogens. For general methods to prepare antibodies, see Antibodies: A Laboratory Manual (1988), E. Harlow and D. Lane, Cold Spring Harbor Laboratory, Cold Spring Harbor, N.Y., incorporated herein by reference.

These antibodies can in turn be used in the detection of specific alleles of ECR1-10 and ERCA-E in samples and in the detection of cells comprising these regulatory elements in complex mixtures of cells. Such detection methods would have application in screening, diagnosing, and modulating related diseases and other conditions, resulting from increased levels of SOST.

In a preferred embodiment, the antibodies that specifically bind to ECR1-10 and ERCA-E or the antibodies that specifically bind of proteins that interact with these regulatory elements are used to inhibit the function of ECR2-8, thereby modulating SOST expression. The present invention provides for carrying out the present method of modulating SOST expression with an antibody to one of the described SOST regulatory elements in a human patient. In one embodiment, the SOST enhancer to be inhibited is ECR5. First, one would first obtain sufficient amounts of an anti-ECR5 antibody appropriate for human use. While the mouse or rat antibodies may be appropriate for human use, more likely one would obtain a humanized or fully human antibody shown to inhibit human ECR5 according to the methods discussed above. This monoclonal antibody would be administered at a dosage level of 1-6 μg/kg, as determined by routine experimentation (see below) to provide any measurable effect on bone density levels. Beginning, e.g., two days after antibody administration, bone density levels are monitored, using methods known in clinical practice. Antibody compositions may be formulated according to known pharmaceutical principles. It may be provided as an oral formulation or an intravenous solution or administered locally via injection or catheterization. In a preferred embodiment, it may be a sterile, clear, colorless liquid of pH 7.0 to 7.4, which may contain a small amount of easily visible, white, amorphous, drug particulates. In another embodiment, a single-use, 50-mL vial may contain 100 mg of anti-integrin antibody at a concentration of 2 mg/mL and be formulated in a preservative-free solution containing 8.4 mg/mL sodium chloride, 0.88 mg/mL sodium phosphate dibasic heptahydrate, 0.42 mg/mL sodium phosphate monobasic monohydrate, and Water for Injection, USP.

Dosages are determined thorough routine experimentation, depending on the potency of the antibody used. They may be below 1 mg, but typically may be expected to range between 20 and 800 mg/m² calculated body surface. For example, a 400 mg/m² initial dosage might be followed by 250 mg/m² weekly doses. Combination therapy may be administered prior to or after each dose.

2. Inhibitor Design

In one embodiment, known methods are used to identify sequences that inhibit SOST regulatory elements and other candidate genes which are related to bone density and digital formation. Such inhibitors may include but are not limited to, peptide inhibitors and aptamer sequences that bind and act to inhibit ECR5 and other SOST regulatory element expression and/or function.

In another embodiment, aptamer sequences which bind to specific RNA or DNA sequences can be made. Aptamer sequences can be isolated through methods such as those disclosed in co-pending U.S. patent application Ser. No. 10/934,856, entitled, “Aptamers and Methods for their Invitro Selection and Uses Thereof,” which is hereby incorporated by reference.

It is contemplated that the sequences described herein may be varied to result in substantially homologous sequences which retain the same function as the original. As used herein, a polynucleotide or fragment thereof is “substantially homologous” (or “substantially similar”) to another if, when optimally aligned (with appropriate nucleotide insertions or deletions) with the other polynucleotide (or its complementary strand), using an alignment program such as BLASTN (Altschul, S. F., Gish, W., Miller, W., Myers, E. W. & Lipman, D. J. (1990) “Basic local alignment search tool.” J. Mol. Biol. 215:403-410), and there is nucleotide sequence identity in at least about 80%, preferably at least about 90%, and more preferably at least about 95-98% of the nucleotide bases.

In another embodiment, using any of the sequences to any of the SOST-specific regulatory elements provided herein, antisense oligonucleotides and si/shRNA oligonucleotides are designed and made using conventional methods as known and practiced in the art. Such methods are also described in Sahu N K, Shilakari G, Nayak A, Kohli D V., Antisense technology: a selective tool for gene expression regulation and gene targeting, Curr Pharm Biotechnol. 2007 October; 8(5):291-304, herein incorporated by reference. Such si/shRNA or antisense oligonucleotides may, for example, be utilized for the purpose of inhibiting ECR5 function or any combination of the ECR1-ECRE (SEQ ID NOS: 1-15) to modulate the activity or production of SOST, or inhibit regulatory proteins that normally associate with SOST-specific regulatory elements and function to stimulate the production and activity of SOST.

3. Drug Screening and Design

In one embodiment, the invention provides for a composition which inhibits the SOST regulatory elements, especially ECR5, in vivo. In a preferred embodiment, the composition is a small molecule, peptide or an aptamer drug that targets SOST-specific regulatory element or regulatory proteins that normally bind to it and stimulate SOST expression and activity.

In addition to modulating the expression of the SOST gene, the present embodiment further contemplates an alternative method for identifying specific agonists/antagonists and activators/repressors using various screening assays known in the art.

A preferred embodiment contemplates screens for small molecule ligands or ligand analogs and mimics, as well as screens for natural ligands that bind to and agonize/antagonize regulatory element activity in vivo or result in lowered or increased expression of SOST and thereby result in increasing or decreasing bone density. For example, natural products libraries can be screened using assays of the invention for molecules that inhibit or block ECR5 activity (or that of any other regulatory sequences described herein). Knowledge of the primary sequence of the various regulatory element allele variants and other structural motifs of the regulatory elements (e.g., amphipathic α-helices), and the similarity of those sequences with domains contained in other proteins, can provide an initial clue to agonists/antagonists of the protein. Identification and screening of agonists/antagonists is further facilitated by determining structural features of the protein, e.g., using X-ray crystallography, neutron diffraction, nuclear magnetic resonance spectrometry, and other techniques for structure determination, as is known in the art. These techniques provide for the rational design or identification of inhibitors of the ECR1-10 and ERCA-E that will inhibit SOST expression and increase bone mass or release from repression as is the case of sclerosteosis, VB and other bone dysplasia that normally suffer from high bone mass and could benefit from a reduction in bone formation.

Another approach uses recombinant bacteriophage to produce large libraries. Using the “phage method” described by Scott and Smith, 1990, Science 249: 386-390 (1990); Cwirla, et al., Proc. Natl. Acad. Sci., 87: 6378-6382 (1990); Devlin et al., Science, 249: 404-406 (1990), very large libraries can be constructed. A second approach uses primarily chemical methods, of which the Geysen method, Geysen et al., Molecular Immunology 23: 709-715 (1986); Geysen et al. J. Immunologic Method 102:259-274 (1987), and the method of Fodor et al. Science 251: 767-773 (1991) are examples. Houghton in U.S. Pat. No. 4,631,211, and Rutter et al., U.S. Pat. No. 5,010,175, describe methods to produce a mixture of peptides that can be tested as agonists or antagonists.

In another aspect, synthetic libraries and the like can be used to screen for ligands that recognize and specifically bind to ECR1-10 and ERCA-E and their variants. In one such example, a phage library can be employed. Phage libraries have been constructed which when infected into host E. coli produce random peptide sequences of approximately 10 to 15 amino acids, Parmley and Smith, Gene, 73: 305-318 (1988), Scott and Smith, Science, 249: 386-249 (1990). Specifically, the phage library can be mixed in low dilutions with permissive E. coli in low melting point LB agar which is then poured on top of LB agar plates. After incubating the plates at 37° C. for a period of time, small clear plaques in a lawn of E. coli will form which represents active phage growth and lysis of the E. coli. A representative of these phages can be absorbed to nylon filters by placing dry filters onto the agar plates. The filters can be marked for orientation, removed, and placed in washing solutions to block any remaining absorbent sites. The filters can then be placed in a solution containing, for example, a radioactive fragment of the SOST regulatory element. After a specified incubation period, the filters can be thoroughly washed and developed for autoradiography.

Plaques containing the phage that bind to the radioactive binding domain can then be identified. These phages can be further cloned and then retested for the ability to bind to any of the SOST regulatory elements and/or their variants. Once the phages have been purified, the binding sequence contained within the phage can be determined by standard DNA sequencing techniques. Once the DNA sequence is known, synthetic peptides can be generated which represent these inhibitor sequences.

The effective peptide(s) can be synthesized in large quantities for use in in vivo models and eventually in humans to inhibit SOST regulatory elements and thereby modulate SOST function and expression. Synthetic peptide production is relatively non-labor intensive, easily manufactured, quality controlled and thus, large quantities of the desired product can be produced quite cheaply. Similar combinations of mass produced synthetic peptides have recently been used with great success. Patarroyo, Vaccine, 10: 175-178 (1990). The peptides may be prepared according to known pharmaceutical technology. They may be administered singly or in combination, and may further be administered in combination with other cardiovascular drugs. They may be conventionally prepared with excipients and stabilizers in sterilized, lyophilized powdered form for injection, or prepared with stabilizers and peptidase inhibitors of oral and gastrointestinal metabolism for oral administration.

Another embodiment is to create a cell system which has the regulatory region of the human SOST gene, including at least one of the SOST regulatory elements, ECR 1-10 and ERCA-E or combinations of these elements, coupled to a reporter gene, such as luciferase, LacZ, or GFP as is known in the art. In a preferred embodiment, the regulatory region would comprise at least once copy of ECR5 and any other element of interest. The reporter gene is positioned at the start of the SOST gene. Candidate drugs are screened against the cell system and scored for their ability to downregulate/upregulate reporter gene expression, specifically for their ability to block or inhibit, enhance or stimulate a SOST regulatory element. These drugs will have use in stimulating or inhibiting bone and cartilage growth and increasing (or decreasing) bone density, according to the findings of the inventors that ECR1-10 and ERCA-E are SOST regulatory elements, specifically ECR5, and thus can modulate SOST expression, as shown by Example 3.

Other high-throughput methods of drug design and discovery are discussed in Landro, J. A. et al., “HTS in the new millennium, the role of pharmacology and flexibility,” J Pharmacol Toxicol Methods. 2000 July-August; 44(1):273-89, describing target identification, reagent preparation, compound management, assay development, high-throughput library screening and other methods for drug discovery and screening, and is hereby incorporated by reference in its entirety.

4. Methods of Treatment

Bone mass density loss or arthritis can be diagnosed using criteria generally accepted in the art for detecting such disorders, including but not limited to X-rays and bone scans. The inhibitors of the SOST regulatory elements should be administered to a patient in an amount sufficient to elicit a therapeutic response in the patient (e.g., increase in bone mass density, decrease in bone fragility, increased strength and reduced brittleness of bones, reduction in SOST expression, prevention of any symptoms or disease markers or alternatively as a therapy for sclerosteosis, VB disease or related osteopetrosis-like disorders). An amount adequate to accomplish any of these responses is defined as a “therapeutically effective dose or amount.”

In another embodiment, a therapeutic dose would be used not only to treat a disease in a patient, but also prevent bone diseases. For example, in one embodiment, in order to prevent osteoporosis in post-menopausal women, a therapeutic dose would be given to middle age or elderly women in addition to or in replacement of hormone replacement therapy.

The inhibitors of the invention can be administered directly to a mammalian subject using any route known in the art, including e.g., by injection (e.g., intravenous, intraperitoneal, subcutaneous, intramuscular, or intradermal), inhalation, transdermal (topical) application, rectal administration, or oral administration.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Administration of the inhibitors of the invention can be in any convenient manner, e.g., by injection, intravenous and arterial stents (including eluting stents), cather, oral administration, inhalation, transdermal application, or rectal administration. In some cases, the inhibitors are formulated with a pharmaceutically acceptable carrier prior to administration. Pharmaceutically acceptable carriers are determined in part by the particular composition being administered (e.g., nucleic acid or polypeptide), as well as by the particular method used to administer the composition. Accordingly, there are a wide variety of suitable formulations of pharmaceutical compositions of the present invention (see, e.g., Remington's Pharmaceutical Sciences, 17^{th} ed., 1989).

The dose administered to a patient, in the context of the present invention should be sufficient to effect a beneficial therapeutic response in the patient over time. The dose will be determined by the efficacy of the particular vector (e.g. peptide or nucleic acid) employed and the condition of the patient, as well as the body weight or surface area of the patient to be treated. The size of the dose also will be determined by the existence, nature, and extent of any adverse side-effects that accompany the administration of a particular peptide or nucleic acid in a particular patient.

In determining the effective amount of the vector to be administered in the treatment or prophylaxis of diseases or disorder associated with bone density loss, the physician evaluates circulating plasma levels of the inhibitor drug, inhibitor drug toxicities, progression of the disease (e.g., degree of osteoporosis), and the production of antibodies that specifically bind to the inhibitor drug.

Typically, the dose equivalent of a polypeptide is from about 0.1 to about 50 mg per kg, preferably from about 1 to about 25 mg per kg, most preferably from about 1 to about 20 mg per kg body weight. In general, the dose equivalent of a naked nucleic acid is from about 1 μg to about 100 μg for a typical 70 kilogram patient, and doses of vectors which include a viral particle are calculated to yield an equivalent amount of therapeutic nucleic acid.

For administration, SOST regulatory element inhibitors or inhibitors of SOST-regulatory proteins specific to the regulatory elements described herein of the present invention can be administered at a rate determined by the LD-50 of the inhibitor drug, and the side-effects of the drug at various concentrations, as applied to the mass and overall health of the patient. Administration can be accomplished via single or divided doses, e.g., doses administered on a regular basis (e.g., daily) for a period of time (e.g., 2, 3, 4, 5, 6, days or 1-3 weeks or more), or regular long-term use.

Bone loss tends to increase with age. Thus, it is contemplated that treatment with the inhibitors of the present invention may increase bone mass density, to offset continual or increased bone loss as the individual ages, that periodic treatment with the inhibitor may be needed. For example, an individual may need a higher therapeutically effective amount to increase bone mass density to a preferred range wherein there is a lesser danger of fracture, and then once that range of bone mass density is achieved, the administered dose would be lowered to match that of the rate the individual's body breaks down bone so that bone mass density is maintained.

a. Injectable Delivery

In certain circumstances it will be desirable to deliver the pharmaceutical compositions comprising inhibitor drugs to the SOST regulatory elements disclosed herein, parenterally, intravenously, intramuscularly, or even intraperitoneally as described in U.S. Pat. No. 5,543,158; U.S. Pat. No. 5,641,515 and U.S. Pat. No. 5,399,363. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Investigators should select a formulation best suited to the injection route and animal employed for the study. Lyophilized oligonucleotides are readily soluble in aqueous solution and can be resuspended at concentrations as high as 2.0 mM. However, viscosity of the resultant solutions can sometimes affect the handling of such concentrated solutions.

Oligonucleotides can be administered via bolus or continuous administration using an ALZET mini-pump (DURECT Corporation). Caution should be observed with bolus administration as studies of antisense oligonucleotides demonstrated certain dosing-related toxicities including hind limb paralysis and death when the molecules were given at high doses and rates of bolus administration. Studies with antisense and ribozymes have shown that the molecules distribute in a related manner whether the dosing is through intravenous (IV), subcutaneous (sub-Q), or intraperitoneal (IP) administration. For most published studies, dosing has been conducted by IV bolus administration through the tail vein. Less is known about the other methods of delivery, although they may be suitable for various studies. Any method of administration will require optimization to ensure optimal delivery and animal health.

For bolus injection, dosing can occur once or twice per day. The clearance of oligonucleotides appears to be biphasic and a fairly large amount of the initial dose is cleared from the urine in the first pass. Dosing should be conducted for a fairly long term, with a one to two week course of administration being preferred. This is somewhat dependent on the model being examined, but several metabolic disorder studies in rodents that have been conducted using antisense oligonucleotides have required this course of dosing to demonstrate clear target knockdown and anticipated outcomes.

b. Implanted Devices

In some embodiments implanted devices (e.g., arterial and intravenous stents, including eluting stents, and catheters) are used to deliver the formulations comprising the SOST regulatory element inhibitors of the invention. For example, aqueous solutions comprising the peptides and nucleic acids of the invention are administered directly through the stents and catheters. In some embodiments, the stents and catheters may be coated with formulations comprising the peptides and nucleic acids described herein. In some embodiments, the peptides and nucleic acids will be in time-release formulations an eluted from the stents. Suitable stents are described in, e.g., U.S. Pat. Nos. 6,827,735; 6,827,735; 6,827,732; 6,824,561; 6,821,549; 6,821,296; 6,821,291; 6,818,247; 6,818,016; 6,818,014; 6,818,013; 6,814,749; 6,811,566; 6,805,709; 6,805,707; 6,805,705; 6,805,704; 6,802,859; 6,802,857; 6,802,856; and 49 6,802,849. Suitable catheters are described in, e.g., U.S. Pat. Nos. 6,829,497; 6,827,798; 6,827,730; 6,827,703; 6,824,554; 6,824,553; 6,824,551; 6,824,532; and 6,819,951.

c. Particle Delivery and Liposomes.

In certain embodiments, the inventors contemplate the use of liposomes, nanocapsules, microparticles, microspheres, lipid particles, vesicles, and the like, for the administration of the SOST regulatory element inhibitors of the present invention. In particular, the compositions of the present invention may be formulated for delivery either encapsulated in a lipid particle, a liposome, a vesicle, a nanosphere, or a nanoparticle or the like.

The formation and use of liposomes is generally known to those of skill in the art (see for example, Couvreur et al., Nanocapsules: A New Type of Lysosomotropic Carrier, Febs Letters, 84(2): 323-326, (1977); Lasic Novel applications of liposomes, Trends in Biotechnology 16(7): 307-321, (1998); which describes the use of liposomes and nanocapsules in the targeted antibiotic therapy for intracellular bacterial infections and diseases). Recently, liposomes were developed with improved serum stability and circulation half-times (Gabizon & Papahadjopoulos, Liposome formulations with prolonged circulation time in blood and enhanced uptake by tumors. Proc. Natl. Acad. Sci. USA 85: 6949-6953, (1988); Allen and Choun, Large unilamellar liposomes with low uptake into the reticuloendothelial system, FEBS Lett., 223: 42-46, (1987); U.S. Pat. No. 5,741,516). Further, various methods of liposome and liposome like preparations as potential drug carriers have been reviewed (Takakura et al., Biological effects and cellular uptake of c-myc antisense oligonucleotides and their cationic liposome complexes. J. Drug Targeting, 5(4): 235-246 (1998); Chandran, S. et al, Recent Trends in Drug Delivery Systems: Liposomal Drug Delivery System—Preparation and Characterization; Indian J of Experimental Biology, 35: 801-809, (1997); R. Margalit, Liposome-mediated drug targetting in topical and regional therapies. Crit. Rev. Ther. Drug Carrier Syst. 12: 233-261, (1995); U.S. Pat. No. 5,567,434; U.S. Pat. No. 5,552,157; U.S. Pat. No. 5,565,213; U.S. Pat. No. 5,738,868 and U.S. Pat. No. 5,795,587).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

Targeting is generally not a limitation in terms of the present invention. However, should specific targeting be desired, methods are available for this to be accomplished. For example, antibodies may be used to bind to the liposome surface and to direct the liposomes and its contents to particular cell types. Carbohydrate determinants (glycoprotein or glycolipid cell-surface components that play a role in cell-cell recognition, interaction and adhesion) may also be used as recognition sites as they have potential in directing liposomes to particular cell types.

Alternatively, the invention provides for pharmaceutically-acceptable nanocapsule formulations of the compositions of the present invention. Nanocapsules can generally entrap compounds in a stable and reproducible way (Henry-Michelland et al. Attachment of antibiotics to nanoparticles: preparation, drug-release and antimicrobial activity in vitro, Int. J. Pharm. 35, 121-27, 1987; Quintanar-Guerrero et al. Pseudolatex preparation using a novel emulsion-diffusion process involving direct displacement of partially water-miscible solvents by distillation. Int'l J. Pharmaceutics 188(2), 155-64, 1998; Douglas et al. Nanoparticles in drug delivery. Rev. Ther. Drug Carrier Syst. 3, 233-61, 1987). To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use in the present invention. Such particles may be are easily made, as described (Couvreur et al., Tissue distribution of antitumor drugs associated with polyalkylcyanoacrylate nanoparticles. J. Pharm. Sci. 69, 199, 1980; zur Muhlen et al. Solid lipid nanoparticles (SLN) for controlled drug delivery—Drug release and release mechanism. Euro. J. Pharmaceutics and Biopharmaceutics 45(2), 149-55, 1998; Zambaux et al Influence of experimental parameters on characteristics of poly(lactic acid) nanoparticles prepared by a double emulsion method. J. Controlled Release 50(1-3), 31-40, 1998; H. Pinto-Alphandry, A. Andremont and P. Couvreur, Targeted delivery of antibiotics using liposomes and nanoparticles: research and applications. Int. J. Antimicrob. Agents 13, 155-168, (2000); U.S. Pat. No. 5,145,684).

In another embodiment, it is contemplated that such particles can be used to deliver inhibitory peptides and oligonucleotides of the invention for therapeutic applications. For example, Gary D J, Puri N. Won Y Y. Polymer-based siRNA delivery: perspectives on the fundamental and phenomenological distinctions from polymer-based DNA delivery, J Control Release. 2007 Aug. 16; 121(1-2):64-73. Epub 2007 May 26, describe methods and polymers known in the art for delivery of antisense oligonucletides and gene therapy.

d. Other Methods of Delivery

The SOST regulatory element inhibitors, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

In certain applications, the pharmaceutical compositions comprising the SOST regulatory element inhibitors disclosed herein may be delivered via oral administration to the individual. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (Mathiowitz, E., Jacob, J. S., Jong, Y. S., Carino, G. P., Chickering, D. E., Chaturvedi, P., Santos, C. A., Vijayaraghavan, K., Montgomery, S., Bassett, M. and Morrell, C., Biologically erodable microspheres as potential oral drug delivery systems. Nature 386: 410-414, (1997); S. J. Hwang, H. Park and K. Park, Gastric retentive drug-delivery systems, Crit. Rev. Ther. Drug Carrier Syst. 15:243-284, (1998); U.S. Pat. No. 5,641,515; U.S. Pat. No. 5,580,579 and U.S. Pat. No. 5,792,451). The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

5. Recombinatorial Vectors and Constructs and Gene Therapy

The preferred embodiment also encompasses uses of the SOST regulatory elements for gene therapeutics. See Gabor M. Rubanyi, “The future of gene therapy,” Molecular Aspects of Medicine 22 (2001): 113-142 and Ulrich-Vinther M., Gene therapy methods in bone and joint disorders. Evaluation of the adeno-associated virus vector in experimental models of articular cartilage disorders, periprosthetic osteolysis and bone healing, Acta Orthop Suppl. 2007 April; 78(325): 1-64. Review, which are hereby incorporated by reference. Rubanyi describes existing and future methods of gene therapy and the technical hurdles gene therapy faces in the future. Ulrich-Vinther describes the use of gene therapy to treat various bone diseases and the development of gene therapeutic treatment options for complex orthopaedic diseases. The latter study represents proof-of-principle that the rAAV vector promotes efficient gene transfer in vitro to a spectrum of cells with orthopaedic relevance, and that in vivo targeting of somatic tissue with a single administration of a rAAV vector at the time of surgery could be sufficient for long-term expression of therapeutic proteins, thus enabling long-term therapeutic applications using or inhibiting the presently described SOST-specific regulatory elements.

Other examples are drug therapies aimed at lowering the levels of SOST in any human patient with bone disorders. These will provide a suitable way to reduce SOST levels and thereby reduce the risk of bone disease.

Various types of gene delivery vectors can be used including, but definitely not limited to, plasmids, YACs (Yeast Artificial Chromosomes), BACs (Bacterial Artificial Chromosomes), bacterial vectors, bacteriophage vectors, viral vectors (for example, retroviruses, adenoviruses and viruses commonly used for gene therapy), non-viral synthetic vectors, and recombinant vectors. Delivery of the vector and/or construct for gene therapy in a preferred embodiment is by viral infection or injection intravenously although delivery can be by any other means as described previously.

The present embodiment further encompasses a recombinant vector comprising a polynucleotide that is substantially inhibits the SOST regulatory element polynucleotides described herein. Within some embodiments, the expression vectors are employed in the in vivo expression of ECR1-10 or ECRA-E inhibitors in non-human animals. In other embodiments, the expression vectors are used for constructing transgenic animals and gene therapy.

Depending on the host organism or cell wherein the ECR inhibitor/stimulator will be expressed, one skilled in the art can adapt the recombinant vector to further comprise genetic elements, including but not limited to, an origin of replication in the desired host, suitable promoters and regulatory elements, any necessary ribosome binding sites, polyadenylation signal, splice donor and acceptor sites, transcriptional termination sequences, selectable markers and non-transcribed flanking sequences. Various types of gene delivery vectors can be used including, but definitely not limited to, plasmids, YACs (Yeast Artificial Chromosomes), BACs (Bacterial Artificial Chromosomes), bacterial vectors, bacteriophage vectors, viral vectors (for example, retroviruses, adenoviruses and viruses commonly used for gene therapy), non-viral synthetic vectors, and recombinant vectors, etc.

One embodiment comprises a host cell that has been transformed or transfected with a non-functional variant of one of the ECR1-10 and ERCA-E polynucleotides described herein. In a preferred embodiment, the host cell has been transformed with a polynucleotide comprising a mutant non-functional SEQ ID NO: 5 or a fragment or variant thereof. Appropriate host cells can be prokaryotic host cells, such as E. coli, Bacillus subtilis, Salmonella typhimurium, and strains from species including but not limited to, Pseudomonas, Streptomyces and Staphylococcus. Alternatively eukaryotic host cells can be used, including but not limited to, HeLa cells, HepG2 and other mammalian host cells. In another embodiment a mammalian host cell comprises the SOST and/or its regulatory elements genomic region, wherein the regulatory elements are disrupted by homologous recombination with a knockout vector.

In order to study the physiological and phenotypic consequences of a lack of synthesis of the Sost protein, both at the cellular level and at the organism level, the preferred embodiment also encompasses DNA constructs and recombinant vectors enabling conditional expression of the SOST genomic sequence, including the SOST gene as described in SEQ ID NO: 16, and including all or a portion of the sequences set forth in SEQ ID NOS: 1-15 and 17-59, more preferably SEQ ID NOS: 1-15, in a transgenic non-human animal.

The targeting construct can be built by various methods known in the art including but not limited to, PCR primers for integration by homologous recombination, using a repressor/marker promotor construct, Cre-LoxP system, and antisense constructs. The method preferred is using PCR products and primers to build the targeting construct. To build such a construct to make knockout non-human animals and cells, one would need the homology “arms” that flank each side of the sequence to be deleted or disrupted, and a selectable marker inserted between the arms to select for the marker function. The sequence to be deleted can be the 52 kb region missing in VB patients as the inventors did in Example 1, parts of the VB deletion region, the SOST gene or parts of SOST, or any of the SOST regulatory elements, single or multiple exons, introns, intervening genomic sequences up to the nearest neighboring gene on each side, short peptide sequences and even single base pair deletions. After delivery of the construct into embryonic stem cells, selection for the marker permits gene deletion. Or for instance, SOST gene function can be disrupted by insertion of the selectable marker, by insertion of the marker in the promoter, splice sites, or the open reading frame.

In order to effect expression of the polynucleotides and polynucleotide constructs of the preferred embodiment, these constructs must be delivered to the host cell, where once it has been delivered to the cell, it may be stably integrated into the genome of the host cell and effectuate cellular expression. This delivery can be accomplished in vitro, for laboratory procedures for transforming cell lines, or in vivo or ex vivo, for the creation of therapies or treatments of diseases. Mechanisms of delivery include, but are not limited to, viral infection (where the expression construct is encapsulated in an infection viral particle), other non-viral methods known in the art such as, calcium phosphate precipitation, DEAE-dextran, electroporation, direct micro-injection, DNA-loaded liposomes, and receptor-mediated transfection of the expression construct. In a preferred embodiment, the delivery of the construct is by micro-injection into the appropriate host cell or by intravenous injection in the organism.

One embodiment is modelled after the methods described by Kumar S, Ponnazhagan S, Gene therapy for osteoinduction, Curr Gene Ther. 2004 September; 4(3):287-96, which describes existing therapies for osteoinduction and discusses the potential and limitation of vector-mediated gene therapy for bone diseases. The preferred embodiment contemplates similar protocols of gene transfer as described in Kumar et al. based on the same target tissues and the desire to express SOST regulatory elements and their mutants, variants and inhibitors endogenously.

A second-generation recombinant adenovirus encoding an inhibitor of ECR5 can be constructed using methods as described by Tsukamoto K. et al., Journal of Lipid Research, 1997:38, 1869-1876. Briefly, pAdCMV ECR5 inhibitor encoding sequence can be linearized with an enzyme and co-transfected into cells along with adenoviral DNA isolated and digested. The cells are then overlaid with agar and incubated at 32° C. for about 15 days. Plaques positive for the inhibitor are subjected to a second round of plaque purification, and the recombinant adenovirus is then expanded in cells at 32° C. A null adenovirus can be constructed and expanded in an identical manner. All viruses are then purified and stored appropriately.

While much of gene therapy uses vectors as a means of delivery, other methods of delivery to the somatic cells of a patient may be utilized. The preferred embodiment also contemplates the delivery of ECR2-8 inhibitor polynucleotides by encapsulation by compositions such as, hydrogels and microgels, liposomes, and other lipid or polymer carriers. Furthermore, the inhibitor polynucleotides can be delivered naked, without any means of receptor-mediated entry or other carrier into the patient's cells.

In another embodiment, the invention provides for methods of delivering a SOST regulatory element that is non-functional to replace the functional element in vivo or removing/deleting a SOST enhancer element such as ECR5 in vivo.

6. Combination Therapeutics Using SOST Regulatory Element Inhibitors

The presently described SOST regulatory element inhibitory polynucleotides, polypeptides, small molecules and drugs may be prepared according to known pharmaceutical technology. They may be administered singly or in combination, and may further be administered in combination with other cardiovascular or triglyceride-lowering drugs. They may be conventionally prepared with excipients and stabilizers in sterilized, lyophilized powdered form for injection, or prepared with stabilizers and peptidase inhibitors of oral and gastrointestinal metabolism for oral administration. They may also be administered by methods including, but not limited to, intravenous, infusion, rectal, inhalation, transmuscosal or intramuscular administration.

It is contemplated that the inhibitors of each of the described SOST enhancers and other regulatory elements are used singly or in combination. Furthermore, it is contemplated that these inhibitors are used in conjunction with current bone disease therapeutics including, but not limited to, vitamin D, calcium and other vitamin supplements, treatment with osteoinductive growth factors and proteins, calcitonin, PTH, and biphosphonates.

It is also contemplated that combining data from stratification and genetic studies with diagnostic tests to determine the best method of treatment for person based upon such criteria as specific genotype, age, gender and ethnicity. For example, after finding in a genetic study that individuals having increased levels of SOST or ECR5 and a specified bone density level, and an observed response to a certain dosage of the ECR5 inhibitor (e.g. their bone mass levels dramatically are increase), physicians and medical providers can tailor bone disease therapy to prescribe the most effective dosage of SOST or ECR5 lowering medication.

### Example 1

### Molecular Characterization of Van Buchem

### Transgenic Mouse Models

A ˜158 kb human BAC (RP11-209M4) (SOST^{wt}) encompassing the 3′ end of the DUSP3 gene, SOST, MEOX1, and 90 kb noncoding intergenic interval separating SOST from the MEOX1 neighboring gene was engineered using homologous recombination in bacteria (Lee, E. C., D. Yu, J. Martinez de Velasco, L. Tessarollo, D. A. Swing, D. L. Court, N. A. Jenkins, and N. G. Copeland. 2001. A highly efficient Escherichia coli-based chromosome engineering system adapted for recombinogenic targeting and subcloning of BAC DNA. Genomics 73: 56-65) to delete the 52 kb region missing in VB patients and to create the VB (SOST^{wbΔ}) allele (FIG. 1A). These constructs were used to generate several lines of transgenic mice. Similar to the endogenous mouse SOST expression, and reported human expression SOST^{wt} transgenic animals predominantly expressed the human SOST transcript in the mineralized bone of neonatal and adult mice. In adult tissues, we detected appreciable amounts of human SOST in the brain, heart, lung and kidney. Similar human SOST expression was detected in the heart and kidney of SOST^{wbΔ} transgenic mice, but was dramatically reduced in the bone, brain and lung (FIG. 1B). These data demonstrate that in vivo, the VB allele confers dramatically reduced SOST expression in the adult bone and suggests that the vbΔ deletion contains an essential bone enhancer element.

Generating Transgenic Mice. FRT-kan-FRT cassette was excised from pICGN21 vector (KpnI; SacI) and inserted into pUC18 to create pUC18.kan.FRT. Homologous arms were PCR-amplified from 209M4 BAC DNA and cloned into pUC18.kan.FRT vector using EcoRI/SacI sites for the left arm (VBDelH1: fwd 5′-TTGGTACCGGATTGAAGTGATCCCCAGCTGGA-3′ (SEQ ID NO: 81); rvd 5′-TTGAGCTCCAATCTCCTGACCTTGTGATCCGC-3′ (SEQ ID NO: 82), and the SmaI site for the right arm (VbDelH2: fwd 5′-TTCCCGGGCGCTTGAACCCAGTAGGTGGAGG-3′ (SEQ ID NO: 83); rvd 5′-TTCCCGGGTACCAAGGGATGGACAGAAGACAGGCAG-3′ (SEQ ID NO: 84)) to create the recombination vector pUC18.kan.FRT.VBDel. 200-300 ng KpnI digested VBDelH1-FRT-kan-FRT-VbdelH2 fragment was electroporated into EL250-209M4 cells. Recombinant BACs were identified by PCR and pulse-field gel analysis, were isolated at a final concentration of 1 ng/ml and microinjected into fertilized FVB mouse eggs using standard procedures. Transgenic mice were genotyped using PCR analysis of DNA prepared from tail DNA of founder animals using the following primer pair: 5′-ATGTCCACCTTGCTGGACTC-3′ (SEQ ID NO: 85) and 5′-GTCTGTGGGCTGGTTTGCAT-3′ (SEQ ID NO: 86). Transgenic mice were maintained on FVB background.

SOST is an osteocyte-expressed negative regulator of bone formation that is structurally most closely related to the DAN/Cerberus family of BMP antagonists. Several members of this family including noggin and gremlin are expressed embryonically in the developing limb), therefore we examined human SOST expression in the early mouse embryo. rtPCR analysis of RNA isolated from whole embryos showed high levels of human SOST expression in both SOST^{wt} and SOS^{wbΔ} transgenic animals (FIG. 1C). SOST expression precedes endochondral ossification, and was detected as early as E10.5. Since VB deletion did not impact human SOST embryonic expression, we used E10.5 embryonic RNA to quantify the level of transgene expression in different SOST^{wt} and SOST^{wbΔ} transgenic founder lines (FIG. 1D). Comparable expression levels were also confirmed in the kidneys of SOST^{wt} and SOST^{wbΔ} animals. This data strengthens the evidence that the lack of human SOST bone expression in hSOST^{wbΔ} animals is dependent on the 52 kb noncoding deletion, rather than reflecting an artifact due to transgene copy number integration.

RT-PCR, Quantitative RT-PCR and in situ hybridization. Total RNA was isolated with Trizol reagent (Invitrogen) and reverse-transcribed into cDNA (Superscript II, Gibco) using standard methods. cDNA was amplified using GC-Melt PCR kit (Clontech; 65° C. annealing/3 min extension/35 cycles] using human (fwd 5′-AGAGCCTGTGCTACTGGAAGGTGG-3′ (SEQ ID NO: 87), rvd 5′-TAGGCGTTCTCCAGCTCGGCC-3′ (SEQ ID NO: 88)) and mouse (fwd 5′-GACTGGAGCCTGTGCTACCGA-3′ (SEQ ID NO: 89), rvd 5′-CTTGAGCTCCGACTGGTTGTGGAA-3′ (SEQ ID NO: 90)) SOST primer sets. Mouse beta-actin (fwd 5′-CCTCTATGCCAACACAGTGC-3′ (SEQ ID NO: 91), rvd 5′-CTGGAAGGTGGACAGTGAGG-3′ (SEQ ID NO: 92)) was used as control [58° C. annealing/30 sec extension/25 cycles]. Quantitative rtPCR expression analysis was performed using an ABI Prism 7900HT sequence detection system, TaqMan® Universal PCR Master mix, human 18S rRNA pre-developed TaqMan® assay reagent for normalization and TaqMan® Assay-on-Demand™ products for mouse, rat and human SOST all from Applied Biosystems. We considered noon on the day that we found a vaginal plug to be E0.5. We carried out RNA localization by whole-mount in situ hybridization according to established protocols. RNA antisense probes were labeled with digoxigenin and were synthesized with T7 RNA polymerase as previously described.

### Example 2

### Modulation of SOST Expression Impacts Bone Formation

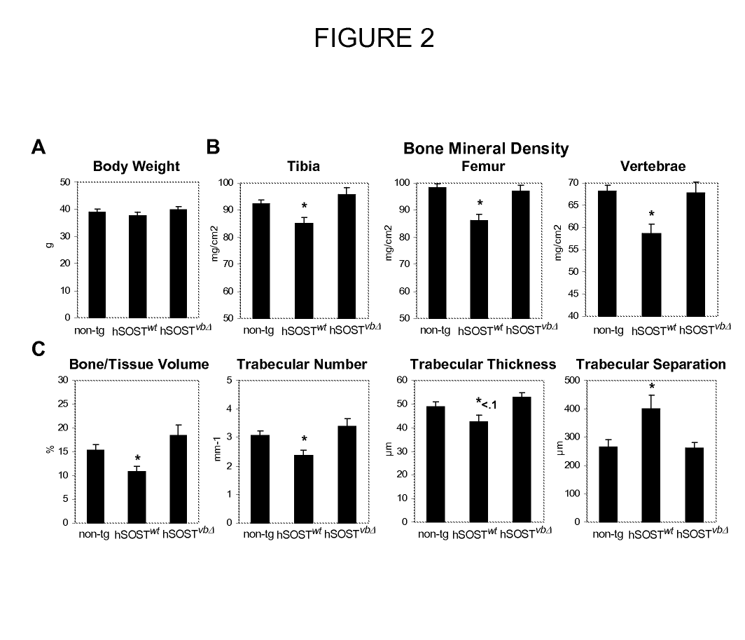

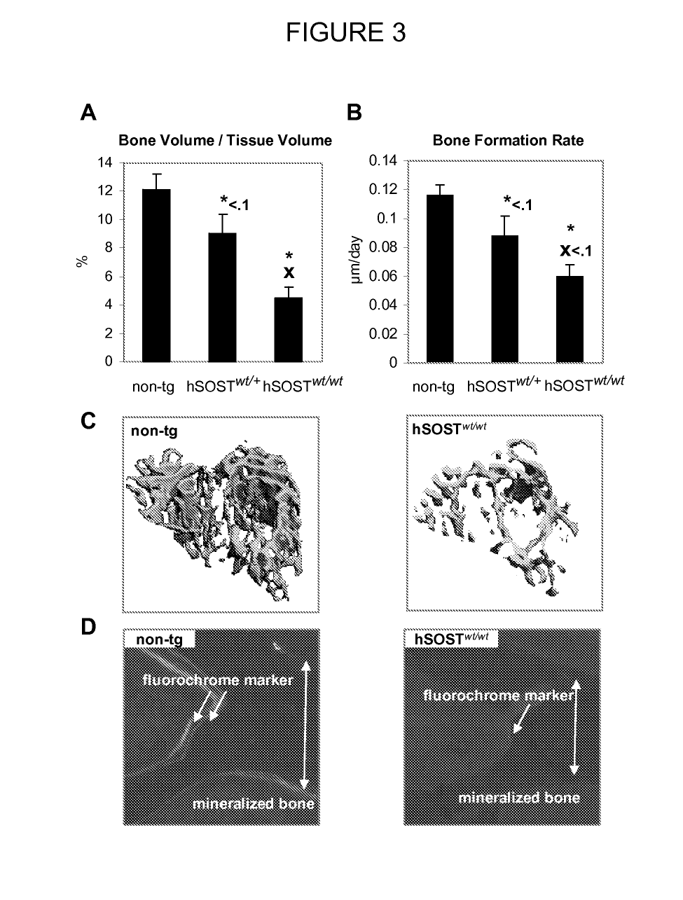

Since lack of SOST causes increased bone density, it was investigated whether elevated levels of human SOST have opposite effects on bone mass. SOST^{wt} transgenics grew to skeletal maturity with normal body size and weight (FIG. 2A) however, the animals displayed decreased bone mineral density in the appendicular and axial skeleton, as evaluated by dual energy X-ray absorptiometry (DEXA) analysis (FIG. 2B). Micro-Computed-Tomography (microCT) analysis of three-dimensional cancellous bone structures revealed that the mice have decreased bone volume, trabecular number, thickness and increased trabecular separation (FIG. 2C). In contrast, the bone parameters of SOST^{wbΔ} transgenics were indistinguishable from non-transgenic littermate controls. The observed osteopenia was gene dose dependent. SOS t transgenic mice bred to homozygosity revealed a further dramatic decrease in tibial cancellous bone volume (FIG. 3A). Histomorphometric analysis revealed that these animals display further decreased bone formation rates at skeletal maturity both in cancellous (FIG. 3B) and cortical bone (tibia: non-tg=0.319+/−0.016 μday versus SOS^{wt/wt}=0.110+/−0.027 μm/day p<0.001) in both the appendicular (FIG. 3B,C) and the axial skeleton. Neither the number of terminally differentiated bone forming cells, the osteocytes, nor the number of bone resorbing cells, the osteoclasts, were significantly affected by the transgene expression (data not shown).

Dual energy X-ray absoptiometry (DEXA) analysis. Tibial, femoral and lumbar vertebral bone mineral density (in milligrams per square centimeter) was measured using a regular Hologic QDR-1000 instrument (Hologic, Waltham, Mass., USA). A collimator with 0.9-cm-diameter aperture and an ultrahigh resolution mode (line spacing, 0.0254 cm; resolution, 0.0127 cm) were used. The excised long bones were placed in 70% alcohol onto a resin platform provided by the company for soft tissue calibration. Daily scanning of a phantom image controlled the stability of the measurements. Instrument precision and reproducibility had been previously evaluated by calculating the coefficient of variation of repeated DEXA and had been found to be below 2%. Coefficients of variation were 0.5 to 2% for all evaluated parameters. A set of 5-month-old male mice was analyzed (non-tg=13 littermates of all analyzed lines, SOST^{wt}=15 (heterozygous mice from 2 SOST^{wt} lines, SOST^{wbΔ}=14 off-springs of heterozygous matings from 2 hSOSTvbD lines).

Micro computed tomography (microCT) analysis. Cancellous bone structure was evaluated in the proximal tibia metaphysis using a Scanco vivaCT20 (Scanco Medical AG, Bassersdorf, Switzerland). The nonisometric voxels had a dimension of 12.5 μm×12.5 μm×12.5 μm. From the cross-sectional images the cancellous bone compartment was delineated from cortical bone by tracing its contour at every 10th section. In all the other slices boundaries were interpolated based on the tracing to define the volume of interest. 660 slices covering a total length of 0.8 mm within the area of the secondary spongiosa (1.3 mm from the proximal end) were evaluated. A threshold value of 175 was used for the three dimensional evaluation of trabecular number, thickness, and separation. Both sets of male 5-month-old mice on which DEXA and histomorphometric analysis has been performed were analyzed. A voxel size of 25 μm×25 μm×25 μm was chosen for visualization of the digits of the fore- and hind limbs.

Histomorphphometric analysis. After dissection, the tibia and lumbar vertebrae were placed for 24 h in Karnovsky's fix, dehydrated in ethanol at 4° C., and embedded in methylmethacrylate. A set of 4- and 8-, microm-thick nonconsecutive microtome sections were cut in the frontal midbody plane for evaluation of fluorochrome-label-based dynamic and cellular parameters of bone turnover. The 4 microm-thick sections were stained with TRAP and Giemsa stain. The sections were examined using a Leica DM microscope (Leica, Glattbrugg, Switzerland) fitted with a camera (SONY DXC-950P, Tokyo, Japan) and adapted Quantimet 600 software (Leica, Cambridge, UK). Two sections/animal were sampled for all sets of parameters. Microscopic images of specimens were evaluated semiautomatically digitally (×400 magnification). All parameters were measured and calculated according to Paritt et al. 1987 (J Bone Min Res). Fluorochrome label bone formation dynamics were evaluated on unstained 8 microm-thick sections. Bone perimeter, single and double labeled bone perimeter, and interlabel width were measured. Mineralized perimeter (%), mineral apposition rates (micrometers/day) (corrected for section obliquity in the cancellous bone compartment), and daily bone formation rates (daily bone formation rate/bone perimeter [micrometer/day]) were calculated. Osteoclast numbers (osteoclast number/bone perimeter [millimeters-1]) and perimeter values (osteoclast perimeter/bone perimeter [percent]) were determined on the TRAP stained slides, and osteocyte number (osteocyte number/bone perimeter [millimeters-1]) on the Giemsa stained slides. All parameters were evaluated in the spongiosa and at the endocortex. A set of 5-month-old male mice from one SOST^{wt} line was analyzed (non-tg=5, SOST^{wt}=7, SOST^{wt}=4).

In general, the osteopenic phenotype we observed is consistent with reports describing transgenic mice overexpressing BMP-antagonists from cDNA constructs driven by osteocalcin (OG2) promoter. The osteopenia phenotypic variation observed between cDNA and BAC SOST transgenic mice is most likely attributed to the transcriptional control of human SOST in each transgenic construct. [symbol] BAC transgenics more faithfully mirror the proper regulatory control exerted on the SOST gene in the endogenous context of the human genome, while the OG2>SOST transgenic expression is ectopic and highlights the transcriptional specificity of the osteocalcin promoter.

In contrast to [symbol] transgenics, [symbol] animals did not display a bone phenotype in neither the appendicular nor the axial mature skeleton, even in the homozygous configuration (FIG. 2B,C). These data demonstrate that modulation of SOST expression dramatically impacts bone formation in the adult mammalian skeleton. Most importantly, these phenotypic data suggest that overexpressing human SOST under the control of its own proximal promoter elements in concert with the downstream VB region negatively modulates adult bone mass. In contrast, bone mass is unaffected in transgenic animals that lack the 52 kb VB region, in a construct that mimics the allele carried by VB patients, consistent with the model that Van Buchem disease is caused by removing a bone-specific regulatory element.

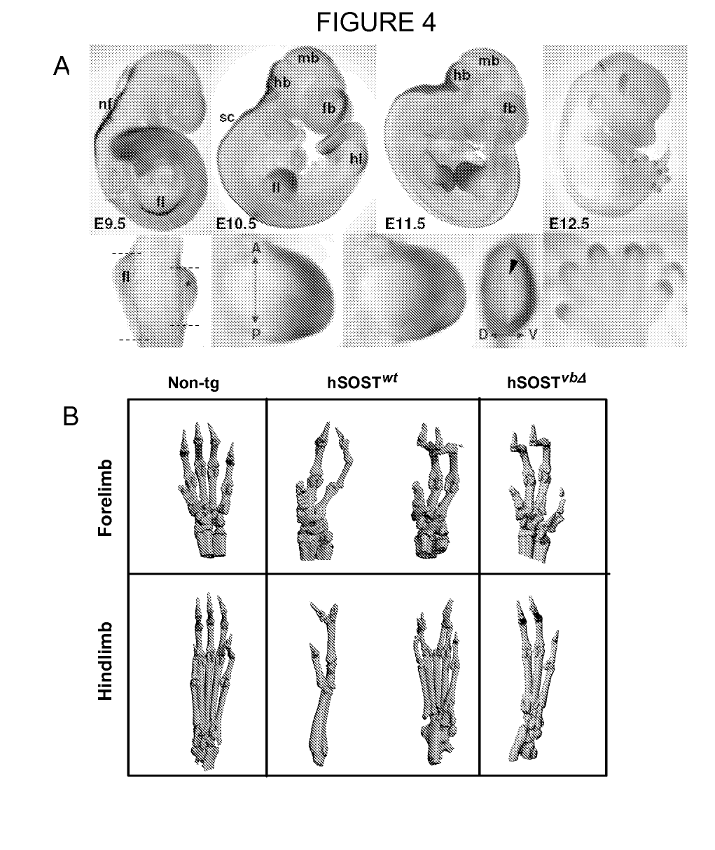

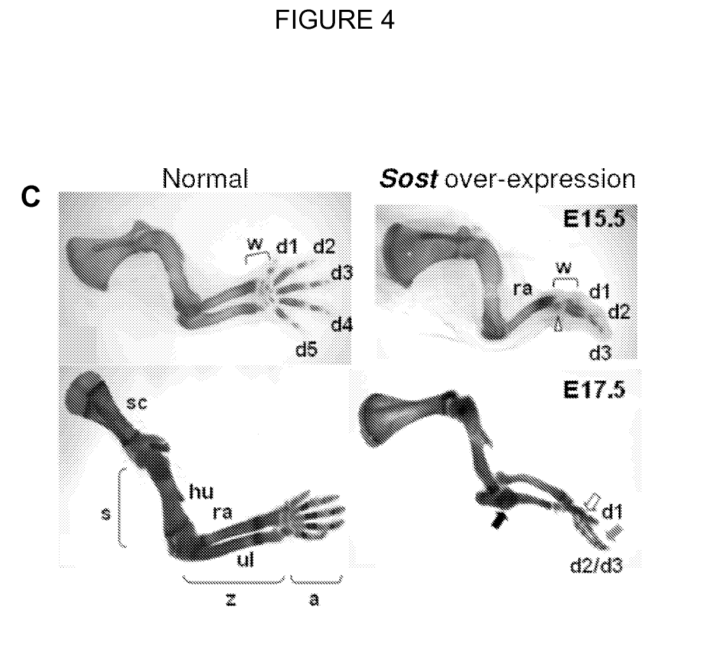

Interestingly, and consistent with the observed embryonic expression, elevated levels of human SOST result in abnormal digit development in both [symbol] and [symbol] BAC transgenics bred to homozygosity. The fore- and hind-limbs of these animals display a wide range of fused and missing digits as visualized by autoradiography (data not shown), ACT (FIG. 4B), and skeletal preps (FIG. 4C). rtPCR data correlates SOST expression with the severity of digit abnormalities (data not shown). Mouse whole mount in situ hybridization revealed SOST to be expressed as early as embryonic stage 9.5 (E9.5), predominantly in the mesenchymal tissue of the developing limb bud (FIG. 4A). These findings imply that SOST embryonic expression is controlled by a transcriptional regulatory element located outside the vbΔ region, consistent with the observation that both sclerosteosis and VB patients suffer from abnormal bone mass accumulation while only sclerosteosis patients exhibit syndactyly of the digits. Since sclerosteosis is caused by SOST null mutations, our results indicate that VB disease and sclerosteosis are allelic, VB patients are hypomorphic for the SOST gene and lack SOST expression in the adult bone. Our data implies that SOST embryonic expression is unaltered in VB patients who never display syndactyly of the digits indicating that both reduced and elevated levels of human SOST negatively impact limb development and digit formation, a novel function attributed to this molecule.

### Example 3

### Comparative Sequence Analysis and In Vitro Enhancer Assays

Given the striking bone phenotypes observed in both VB and sclerosteosis patients, we next focused on the identification of noncoding sequences required for SOST bone-specific expression through a combination of comparative sequence analysis and transient transfections assays. We aligned a ˜140 kb human SOST region (URL:<http://zpicture.dcode.org/>) (Ovcharenko, I., G. G. Loots, R. C. Hardison, W. Miller, and L. Stubbs. 2004. zPicture: dynamic alignment and visualization tool for analyzing conservation profiles. Genome Res 14: 472-477) (RP11-209M4; AQ420215, AQ420216) to the corresponding mouse sequences from chromosome 11 (Mouse chr11:101,489,231-101,688,385; Oct. 3 Freeze). (FIG. 5A). A stringent requirement of at least 80% identity over a 200 base pair (bp) window (≧80% ID; ≧200 bp) identified seven evolutionarily conserved regions (ECR2-8) within the vbΔ genomic interval, which were prioritized for in vitro enhancer analysis. ECR2-8 were tested for their ability to stimulate a heterologous promoter (SV40) in osteoblastic (UMR-106) and kidney (293) derived cell lines. One element, ECR5, was able to stimulate transcription in UMR106 cells (FIG. 5B), but not in the kidney cell line, suggesting that ECR5 enhancer function is specific to the osteoblastic lineage.

We also tested the transcriptional activity of the human SOST proximal promoter region (2 kb region upstream of 5′UTR) in the two cell types and compared it to the SV40 and the osteoblast-specific osteocalcin promoter (OG2). The SV40 promoter showed comparable activity in both cell lines and, as expected, OG2 was only active in the UMR-106 cells. The SOST promoter stimulated transcription in the osteoblastic cells similarly albeit slightly higher activity than the OG2 promoter, while it demonstrated a threefold stronger activity in kidney cells (FIG. 5B).

These data suggest that SOST kidney expression may be due to proximal promoter sequences, whereas strong expression in osteoblast cells requires the activity of the ECR5 enhancer element. Consistent with the results obtained from transfecting SV40 promoter constructs, only ECR5 was capable of activating the human SOST promoter (4×) in UMR106 cells (FIG. 5C). Thus, a small sequence element within the vbA region (ECR5) was identified that confers in vitro osteoblast-specific enhancer activity onto both the human SOST and the SV40 heterologous promoter.

To test ECR5's ability to drive expression in the skeletal structures of the mouse embryo we expressed an ECR-hsp68-LacZ construct in transgenic mice (FIG. 5D) (Nobrega et al. 2003). Transient transgenic animals were created using standard techniques (Mortlock et al. 2003) and F0 pups were stained for β-galactosidase expression at E14.5 (Nobrega et al. 2003). Transgenic embryos expressed LacZ in cartilage of the ribs, vertebrae and skull plates (FIG. 5D). LacZ expression in the adult transgenics was counterstained with bone and cartilage markers, and transgene expression was consistently observed in the skeletal structures. These data confirm that the 250 basepair (bp) ECR5 contained within the 52 kb VB region is indeed a bone specific enhancer in vivo.

In vitro Enhancer Assays. ECRs were PCR-amplified with 5′NheI-linkers, TOPO-cloned into pCR2.1 vector (Invitrogen) then shuttled into NheI/XhoI sites of pGL3-promoter (Promega) or HindIII/PstI of hsp68-LacZ (B. Black). The following primers were used to amplify human DNA (62° C. annealing/30 sec extension/35 cycles):

| | |
|---|---|
| ECR2 (545 bp) | |
| 5′-AGCAACGCAGGGCAGGAGCCAAGA-3′ | (SEQ ID NO: 65) |
| 5′-TAGCTGGCCTCTCCTGGGCGTCTT-3′ | (SEQ ID NO: 66) |
| ECR3(410 bp) | |
| 5′-GGGGGCTGTATGGAAAGGAGACAT-3′ | (SEQ ID NO: 67) |
| 5′-CTTGAGCAGTAGGGCCAAGCCCT-3′ | (SEQ ID NO: 68) |
| ECR4(296 bp) | |
| 5′-TGACAAACAGGAAGGTGGCAGGGC-3′ | (SEQ ID NO: 69) |
| 5′-CCCCCAACATTCCTGTCCCCTTG-3′ | (SEQ ID NO: 70) |
| ECR5(259 bp) | |
| 5′-TCCTTGCCACGGGCCACCAGCTTT-3′ | (SEQ ID NO: 71) |
| 5′-CCCCCTCATGGCTGGTCTCATTTG-3′ | (SEQ ID NO: 72) |
| ECR6(666 bp) | |
| 5′-CCCTGAGAAACATGCCTCTGTCCC-3′ | (SEQ ID NO: 73) |
| 5′-CTTAGCAATCTGGGTGACCCTGGG-3′ | (SEQ ID NO: 74) |
| ECR7(568 bp) | |
| 5′-AAACTGCCAAGCCCCAGCTGGCTA-3′ | (SEQ ID NO: 75) |
| 5′-GCCCAGGGCTCAGAAATGTGTGGA-3′ | (SEQ ID NO: 76) |
| ECR8(352 bp) | |
| 5′-TTCCTACCAAGGTGGCTGCCACC-3′ | (SEQ ID NO: 77) |
| 5′-CCTTCAGAGAAGCAAATGGCTGGGG-3′ | (SEQ ID NO: 78) |
| -2kb promoter | |
| 5′-CAGCAGAAGATGTCACAGCAGG-3′ | (SEQ ID NO: 79) |
| 5′-GAGCTGCATGGTACCAGCCAGA-3′ | (SEQ ID NO: 80) |

Human SOST promoter sequence (2 kb upstream of 5′UTR) was PCR-amplified with SmaI-linkers and transferred into the SmaI site of pGL3basic (Promega). A luciferase reporter plasmid containing mouse osteocalcin (OG2) promoter sequence from −1323 to +10 in pGL3basic was kindly obtained from B. Fournier (Novartis Basel, Switzerland). Reporter plasmids containing ECR-4, -5 or -6 upstream of the human SOST promoter were generated by inserting the ECR elements into the NheI site. Plasmid DNA was isolated using standard endotoxin-free methods (Qiagen). FuGene (Roche) and a CMV-βgal reporter plasmid (Clontech) as internal control were used for transient transfections of rat UMR-106 and human 293 cells. Cells were incubated for 24 hours at 37° C. and luciferase and galactosidase expression were measured using standard assay kits (Promega).

Transient transgenic analysis. 500 mg of DNA was linearized with NotI, followed by CsCl gradient purification and 2-5 ng was used for pronuclear injections of FVB embryos. E10.5-E14.5 embryos were dissected in ice-cold PBS, and were fixed in 4% paraformaldehyde at 4° C. for 1-2 hours and stained for LacZ as described. Transgenic embryos were detected by PCR from tail DNA [fwd 5′-TTTCCATGTTGCCACTCGC-3′ (SEQ ID NO: 93), rvd 5′-AACGGCTTGCCGTTCAGCA-3′ (SEQ ID NO: 94); 55° C. annealing/30 sec extension/25 cycles].

### Example 4

### Identification and Characterization of SOST-Specific Regulatory Elements

In the present invention, we have demonstrated that the 52 kb noncoding deletion present in Van Buchem patients removes a distant SOST-specific regulatory element and therefore Van Buchem disease is hypomorphic to sclerosteosis. There was no clear view in the prior art of how sclerostin promotes osteogenesis, therefore elucidating its transcriptional regulation was key to understanding the interconnection between its expression pattern in osteogenic cells and its mode of action as either a BMP-antagonist, BMP-agonist or through the WNT-pathway.

Cross-species sequence comparisons, in vitro expression and transgenic analysis were coupled to identify regulatory elements controlling gene expression and provide insights into genetic causes of human bone disorders. An elaborate expression pattern is described along with the multitude of regulatory elements that have the potential to positively or negatively impact SOST in a spatial and temporal precise manner. Consistent with this view, the Examples provide robust in vivo evidence for the role of SOST during bone formation, modulation of adult bone mass and for a novel function during limb development and chondrogenesis.

Furthermore, the findings of the present invention showed that SOST embryonic expression is controlled by a transcriptional regulatory element located outside the vbΔ region, consistent with the observation that both sclerosteosis and VB patients suffer from abnormal bone mass accumulation while only sclerosteosis patients exhibit syndactyly of the digits. In contrast to [symbol] transgenics, [symbol] animals did not display a bone phenotype in neither the appendicular nor the axial mature skeleton, even in the homozygous configuration (FIG. 2B,C). These data demonstrate that modulation of SOST expression dramatically impacts bone formation in the adult mammalian skeleton. Most importantly, these phenotypic data suggest that overexpressing human SOST under the control of its own proximal promoter elements in concert with the downstream VB region negatively modulates adult bone mass. In contrast, bone mass is unaffected in transgenic animals that lack the 52 kb VB region, in a construct that mimics the allele carried by VB patients, which is consistent with the model that Van Buchem disease is caused by removing a distant bone-specific regulatory element. This Van Buchem deletion is herein referred to as the “VB deletion” and is characterized as a deletion of 52 kb region, mapped to chr17:39, 100,192-39,152,480 on the Human Genome May 2004 assembly from UCSC Genome Browser (URL:<http://genome.ucsc.edu/>).

Given the striking bone phenotypes observed in both VB and sclerosteosis patients, the identification of noncoding sequences required for SOST bone-specific expression was carried out through a combination of comparative sequence analysis and transient transfections assays.

An alignment of the ˜140 kb human SOST region—(RP11-209M4; GenBank Accession Nos. AF326736, AQ420215, AQ420216, AF397423) (URL:<http://zpicture.dcode.org/>) was made to the corresponding mouse sequences from chromosome 11 (Mouse chr11:101,489,231-101,688,385; Oct. 3 Freeze) (GenBank Accession No. AF326737) (FIG. 5A). The above GenBank sequences, AQ420215, AQ420216, AF326736 and AF326737 are hereby incorporated by reference. The human SOST gene is SEQ ID NO: 16, GenBank Accession No: NM_{—}025237, which is also hereby incorporated by reference.

A stringent requirement of at least 80% identity over a 200 base pair (bp) window (≧80% ID; ≧200 bp) identified seven evolutionarily conserved regions (ECR2-8) within the vbΔ genomic interval, which were prioritized for in vitro enhancer analysis. ECR2-8 were tested for their ability to stimulate a heterologous promoter (SV40) in osteoblastic (UMR-106) and kidney (293) derived cell lines.

The sequence alignment of the fifteen enhancers identified from human, mouse and other organisms and their percent identity are shown in the attached Sequence listing. The sequence alignment of ECR5 from human, chicken, rat, mouse, opossum and dog is shown. The sequence of the fifteen enhancers from human, human SOST cDNA and the SOST promoter are as follows:

SEQ ID NO: 16
>gi|61676080|ref|NM_025237.2| Homo sapiens sclerosteosis (SOST), mRNA
AGAGCCTGTGCTACTGGAAGGTGGCGTGCCCTCCTCTGGCTGGTACCATGCAGCTCCCACTGGCCCTGTG
TCTCGTCTGCCTGCTGGTACACACAGCCTTCCGTGTAGTGGAGGGCCAGGGGTGGCAGGCGTTCAAGAAT
GATGCCACGGAAATCATCCCCGAGCTCGGAGAGTACCCCGAGCCTCCACCGGAGCTGGAGAACAACAAGA
CCATGAACCGGGCGGAGAACGGAGGGCGGCCTCCCCACCACCCCTTTGAGACCAAAGACGTGTCCGAGTA
CAGCTGCCGCGAGCTGCACTTCACCCGCTACGTGACCGATGGGCCGTGCCGCAGCGCCAAGCCGGTCACC
GAGCTGGTGTGCTCCGGCCAGTGCGGCCCGGCGCGCCTGCTGCCCAACGCCATCGGCCGCGGCAAGTGGT
GGCGACCTAGTGGGCCCGACTTCCGCTGCATCCCCGACCGCTACCGCGCGCAGCGCGTGCAGCTGCTGTG
TCCCGGTGGTGAGGCGCCGCGCGCGCGCAAGGTGCGCCTGGTGGCCTCGTGCAAGTGCAAGCGCCTCACC
CGCTTCCACAACCAGTCGGAGCTCAAGGACTTCGGGACCGAGGCCGCTCGGCCGCAGAAGGGCCGGAAGC
CGCGGCCCCGCGCCCGGAGCGCCAAAGCCAACCAGGCCGAGCTGGAGAACGCCTACTAGAGCCCGCCCGC
GCCCCTCCCCACCGGCGGGCGCCCCGGCCCTGAACCCGCGCCCCACATTTCTGTCCTCTGCGCGTGGTTT
GATTGTTTATATTTCATTGTAAATGCCTGCAACCCAGGGCAGGGGGCTGAGACCTTCCAGGCCCTGAGGA
ATCCCGGGCGCCGGCAAGGCCCCCCTCAGCCCGCCAGCTGAGGGGTCCCACGGGGCAGGGGAGGGAATTG
AGAGTCACAGACACTGAGCCACGCAGCCCCGCCTCTGGGGCCGCCTACCTTTGCTGGTCCCACTTCAGAG
GAGGCAGAAATGGAAGCATTTTCACCGCCCTGGGGTTTTAAGGGAGCGGTGTGGGAGTGGGAAAGTCCAG
GGACTGGTTAAGAAAGTTGGATAAGATTCCCCCTTGCACCTCGCTGCCCATCAGAAAGCCTGAGGCGTGC
CCAGAGCACAAGACTGGGGGCAACTGTAGATGTGGTTTCTAGTCCTGGCTCTGCCACTAACTTGCTGTGT
AACCTTGAACTACACAATTCTCCTTCGGGACCTCAATTTCCACTTTGTAAAATGAGGGTGGAGGTGGGAA
TAGGATCTCGAGGAGACTATTGGCATATGATTCCAAGGACTCCAGTGCCTTTTGAATGGGCAGAGGTGAG
AGAGAGAGAGAGAAAGAGAGAGAATGAATGCAGTTGCATTGATTCAGTGCCAAGGTCACTTCCAGAATTC
AGAGTTGTGATGCTCTCTTCTGACAGCCAAAGATGAAAAACAAACAGAAAAAAAAAAGTAAAGAGTCTAT
TTATGGCTGACATATTTACGGCTGACAAACTCCTGGAAGAAGCTATGCTGCTTCCCAGCCTGGCTTCCCC
GGATGTTTGGCTACCTCCACCCCTCCATCTCAAAGAAATAACATCATCCATTGGGGTAGAAAAGGAGAGG
GTCCGAGGGTGGTGGGAGGGATAGAAATCACATCCGCCCCAACTTCCCAAAGAGCAGCATCCCTCCCCCG
ACCCATAGCCATGTTTTAAAGTCACCTTCCGAAGAGAAGTGAAAGGTTCAAGGACACTGGCCTTGCAGGC
CCGAGGGAGCAGCCATCACAAACTCACAGACCAGCACATCCCTTTTGAGACACCGCCTTCTGCCCACCAC
TCACGGACACATTTCTGCCTAGAAAACAGCTTCTTACTGCTCTTACATGTGATGGCATATCTTACACTAA
AAGAATATTATTGGGGGAAAAACTACAAGTGCTGTACATATGCTGAGAAACTGCAGAGCATAATACTGCC
ACCCAAAAATCTTTTTGAAAATCATTTCCAGACAACCTCTTACTTTCTGTGTAGTTTTTAATTGTTAAAA
AAAAAAAGTTTTAAACAGAAGCACATGACATATGAAAGCCTGCAGGACTGGTCGTTTTTTTGGCAATTCT
TCCACGTGGGACTTGTCCACAAGAATGAAAGTAGTGGTTTTTAAAGAGTTAAGTTACATATTTATTTTCT
CACTTAAGTTATTTATGCAAAAGTTTTTCTTGTAGAGAATGACAATGTTAATATTGCTTTATGAATTAAC
AGTCTGTTCTTCCAGAGTCCAGAGACATTGTTAATAAAGACAATGAATCATGACCGAAAGAAAAAAAAAA
AAAAAAAAAAAA
ECR1 138bp, 76% ID
SEQ ID NO: 1
> Human
TGAGCTCATTTCCTGGGGCGCGCGCGCCGGGCTATTTCAGCCTGGCGCTGTGCAAACAGG
ACAATTTACTGCGGCCAAAAGGGACCCAAATTACAATCGTATCACAGACAAATATCCGCC
ACGCCAGGTCTCCAGGGGCCAGGAGGGGCCTCTCTCCCGGCGCGGGGGGCGGGCGCGGGG
TCAGGCAGGTCCGCGGGGCTCGGCTCGGCCTCGCCGTGCCCTGATCGGC
ECR2 483 bp; 78.7% ID
SEQ ID NO: 2
> human
CCTGGGCGTCTTGTCCCAAGTACAGAGACCTGGATCCTTTCCCACTCATGTGCAACAGCC
CAAAATTAAAAACAAAAGCCATATTAAAAAACAAAACCAACTTTCTGCCTTAAAATATTG
TGAGCCAGGGGGCAATTAGCAATTATGCTGTATTTTATTATGAGAAGATAGAATTCTAAT
TGGACTGATTTGAATTCCACACACCTCCACAGATTGTTTTGGGAATTAAGGTATCAGTTG
TATCGGTAATTATGGTTTACCATTCAATTACCCCCCCACAGAAAACTGTTAAATTGTCTG
TGACGGGGCTTAAATTTAGCTCAGACCTATGTCCTATGAAGACTGCGCGAGTCAATACAA
GCCATCCGGAAACCACCGGGTGCCCTGTGCCAGGCGGTAATTAGGGGTTGAGGTTTCCAA
AGTTTTACCTGAGACAGCAGGGACAAGTGCCTGGGCTGGGCGTGCTCACGTGGGGGGGCT
TGG
ECR3 360 bp; 78.1% ID
SEQ ID NO: 3
> Human
CTTGAGCAGTAGGGCCAAGCCCTGTTCAGCCTGGGACCAAGTTCCCATCAACAAGGTGGT
CTGGGCAGTGGCCAGCCAGAAAGCAGTAATTACTGTCGAGGTGCAGGGACCCCAGGTAGG
GCCCCCACCTCCCACCTCTGTGTGGGCAGTGAATGGGCCTGCCCCTGGGTAAGGCTGTGT
CAGCAGGCGCCTGCCCACCCCTTGCTGGGTTCCCAGGCCCCTAGAGCCCTCTCGTAATAG
GAGCCATTTGCGCTGTAACCAGTGGGTGACCAGATTTTTAATCTTGGAGACCCCTTGGAT
CCCAGGCGGGAAGTGGGATTTGTCAAATGGGGAGAGGCGGGGCTGTCTGGGAATGCCAGA
ECR4 239 bp; 75.7%
SEQ ID NO: 4
> Human
GTCCCCTTGTTTACTCTCAAGCACCCCCTCCCCCACCCAAGGACCAGGTCTTTGTTTACT
GAGCATCTCAGCGATGAGCTCTCACCCCCTGATTTCATCAATTATAAATGTGCTCGCTAC
TCACCACACGGCAATTTGTGACGGACTGTGGTTTGTGGTGAGAGTAGCACCATCCAAGTT
CACCGCAGCCGCGAGTAGAGATGAGGGTTGGGGCCAGACACAGGGCTGTGGGGGCGGCA
ECR5 268 bp; 79.1% ID
SEQ ID NO: 5

> Human

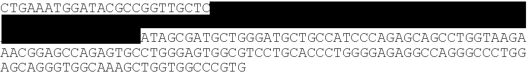

Alignment of highlighted sequence in ECR5 in various organisms

| | | |
|---|---|---|
| SEQ ID NO: 27 | chicken | CT---------------------CCCCATGGCTGCAGCCTCCTTTGTTTTTATTTGACCT |
| SEQ ID NO: 57 | opossum | GT---------------------CTCTGGGGCTGTCTTTTCATTTGCTTTT--TTCATTT |
| SEQ ID NO: 58 | rat | CCTGCCCC------CCTACCTCACCCCAAGCTTG--GTCTCATTTGTTGTC--TTCATTT |
| SEQ ID NO: 26 | mouse | CCTGCCCCACCCCGCCACCCCCACCCCGAGCCTG--GTCTCATTTGTTGTC--TTCATTT |
| SEQ ID NO: 59 | dog | CC---------------------CCTCAAGGCTG--GTCTCATTTGCCTTC--TTCATTT |
| SEQ ID NO: 5 | human | 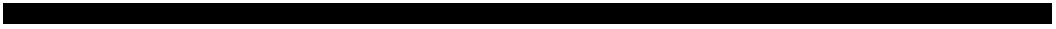 |
| | chicken | TTATAGCGTCGACGC-TTCAGGCCCCTGTGGCAAATTATAACTGTGTTTGCCAGATTGTC |
| | opossum | TTA-------GACACATTCCAAACTTT-CAGCAAATTACA---GTGTTTGCCAACTGGCT |
| | rat | TTA-------GACACATTCCAAACTTTTCAGCAAATTACA---ATGTT-GCCAACTGGCC |
| | mouse | TTA-------GACACATTCCAAACTTTTCAGCAAATTACA---ATGTTTGCCAACTGGCC |
| | dog | TTA-------GACACATTCCAAACTTTTCAGCAAATTACA---GTGTTTGCCAACTGGCC |
| | human | 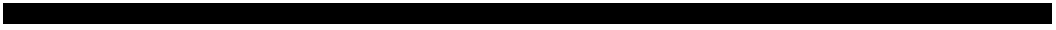 |
| | chicken | GTCTGGGACCGAGGGAAGGAGCTATTT |
| | opossum | GTCTGGGGCCAAGGAGTGGAGTTATTT |
| | rat | GTCTGGGGCCCAGGAGAGA--CTATTT |
| | mouse | GTCTGGGGCTCAGGAGAGA--CTATTT |
| | dog | GTCTGGGGCCCAAGAGAGACACTATTT |
| | human | 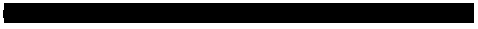 |

ECR6 468 bp; 76.9% ID
SEQ ID NO: 6
> Human
ATTCCATTTCAGGGCCTCTCAGAGTCCTGCCGTGGTGTGCACTGTGTGTGTGTTTAATTT
TCTACATTTGGATGTGATCCTAATCCAATAAATGCTTAGGAGACTTCTATAGAATAGATT
AATTTTTACTAGAAAAAAATATAATTGGCTGATGTTAAGGCTACTGCCCTGACAAATCTG
CCTTGGCCATATATCTGAGAAGGTAAAAGACCCGCTACGCTTGCACATAAATATGCCATC
TTCCCCACAGGCCCTGGAGAAGCACCCCGGGGAGGTTTCCCTTGGTGATTTATTCTTCAT
TAATAAGCTCTATGCTATATTAGGATCAGATTTATGACTCTGCCTTTCTAATATTTCTGA
CATTTCATCTGAAAAGAATTACAAATGAAATCTTGAAACTTTGCCACTTCTCCCTGCTAG
TGCTCTGGCACTCTGTGTCCAAGGGGAGATGGTGGGCTGGGGAGACCC
ECR7 581 bp; 88.1% ID
SEQ ID NO: 7
> Human
TTTCTCTGCCTTTCTAGCTTGGGCCCAGGGCTCAGAAATGTGTGGACTCCCTCACAGCCC
CTCCCAGCATCCCTGCCCCCTCCCAACTGCCTTGGGCAGGTGACACCTGTATTATTGCTA
AGGGTTAAAAAGCCCCCAAATCAATAAAACCCATTAATGAGTGTTGGTACCTCGAAGGCT
ACAGATAAATCCCTTCTACTCAGTGAGTTCAATCCCATAAAACAGCTCTCCCCTTTCAAT
CCTAGCATTCATTTGATAGAAAATGTGGAGAAATTTTAAAAAGGTGACTTACTAATTGCC
TGTAAAATAAAAGGCAGATGGAAGCTTTATTACAGTTGAAGGAAGTCGGGAATATTAAGG
TAAAATGTCAAATAACAATTGATTTTCCTTAGACATAAAGGGGCGATTTATGGCTTCCTA
GTTACTACAAACGAGAAATTATTTGAAGTTCTGAAAAGTATGAGGAGAAATAAAGATTAA
ATAGAAGATGAAATCATAGGGATTTCTCTGGGAGGTGACTTCAGTGCCCCTGGGGACTAG
AATTCATGTGGCCAGTGGCCTAGCCAGCTGGGGCTTGGCAG
ECR8 276 bp; 75% ID
SEQ ID NO: 8
> Human
TGGTGAAAGACACTGCAGAGAAAAGAAAGCACAGCCTGCTGCCCTGGGAATTAACATGAT
TTAGGAGACCTGCAGGTCACCCCCTCATGACTAAAAGCCATCCTGGAATGAAGGTCTGTG
GCTATTTCTAGGCAAAACTGTCTGATAAGATAAAATAGCTCAACTCCTGACCATTAAGTC
GTGAAGGCCATGGCCATCGTAAATCTCATCTTTCCGGCCCTCTGGCCTGCATGCAGTGCA
GCCCAGCCAGTCGGTGGCAGCCACCTTGGTAGGAAG
ECR9 300 bp, 70% ID
SEQ ID NO: 9
> Human
CTGTGAGGTTTATAGTTTCATGACTGTCAGAGCTTTTTAAAATGTGGTAATTTTAAGTGT
GCAGCCTCCCAGGGTCTTTCTTCTTTTAATTGAAGAAATAAACCATCTCCCCTAAGGCAT
GCTTGGCGAAGGAGAAAGGCAGGTGCAAGGCTCACAGAGGAGAGCAGCAGCCTAGAAGGG
CTCTGTGTCATGGGGAAGTAAAACATCCCAGAAACAGAGAGCAGAAGGCCTTGACTGAGC
CCCAGGAGAGGCAGGACAC
ECR10 287 bp, 76% ID (human to mouse)
SEQ ID NO: 10
> Human
TGGAGCCAGCCTGGGAGACTCCCAGCCGCCCACTTCTCGGGGCCTCCCTTTTCCAGCCCC
TTGCTTTCGAGGCAGCAGTGCCATTATTTGGGGAAACCAGCTAACCAGATAGGACAGCAA
ACCGGGGATTTATGTGGTGTGGGAACAGCTCAGGTTTCCCTCCCTGTTTACCCAGCAGTA
TTTTTTAAAACAGAAATCAGCGTGTGGGTAACCGCAGCTGTGAGTTACTAGCTCTGGCTG
TGAGGGCTGGGGTGGGGGGAGTCTCTTCAGAGCCCTCTGTCCACTGG
ECRA 666 bp, 75%
SEQ ID NO: 11
> Human
GAACAGGTGACCTGGAAGATGTTCCTTGGTCCAGTTTCTGCAAACTGCCCCGCAGCATTT
TTAGAAAATGTTCCCTTTTAGATTCGATTTATCTTAGCCAAATTGACCAGGGAAAATAGG
TGCCTACAAATAGCGATCACTGGCAAACAAGGAGAGTTATTATCTTAAATTAAGGCTGGT
TTCTAACAACAAAAAAAAACCCACCAAATGCCACTGGCCACCCCCCACCAACCCCGATCC
CCAGCGCATACGTGAGGACGATGGCTCCACCACCCTCCCTGTAAGTACCAGGCTCAATGC
CGGGCTCTGTGCAAAGGGAAGGAAACAGACAGAGGAAGGAAGGCAAGAGATTAGAGAAGC
TGACAGATGTAAATAGCCTCAGAGGAGCCACACTGTCCCGGCATTTCTCCCCAGGGAGCC
TTTTGTACCAAGGAATCTGGTTGCCTGAAAGAAAAATGTTATCATTTATTTCTTTAGTCA
GAGCTGAGCTGTTTTCTTCAGACATAGAAATAACCTAACATCACACACCAAATTGTTGGC
CAAATGACAGAGATACCTGTAATGTGGGTGTTTAATAATGTCCAGGGTAAACAATCATGG
ACTTGGTTTCTTGGGAAGGGCCCCGTTCCCCGCCACGGTTGCAAAGCTACAGAGGGTCTT
GAATGA
ECRB 320 bp, 77% ID
SEQ ID NO: 12
> Human
GGCTTTAAGGAGCTTGTGGTCACTGAGAGGATTTTGCACTGGAAGTACATGCATTCAAAA
TGGATACCTAAGTGTATATTTTCTGGTGTAAACTATATGTCGACTCTAACTTTAGCCCGG
GGGAGCTTTATTATTTGTCTCCCTTTCATGAAAGCTATAATAGAGGAAGAGAAAACCCTG
CCTCGCACATTCCGATTCCTAAATACATAATTTATAATTTTCTGGGATATTATTTAAGTT
TATTTTAGTTCTGGATACACACCATCCCCGTGGGGTGCTTATTTAAGTATCGGGTGGGCT
CTGGAAAGGCCTGGAATGCC
ECRC 246 bp, 71% ID
SEQ ID NO: 13
> Human
TCTGGGTTCTGGCTGATAAATGGAAATCACCTCCATGGGCCACACAGTAATTAAACTCCT
GGCATTCTTTTGACAAAAAAAAAGTTCCTCATGGGACATTCCCACAAGCTGCTGAAGGTC
TGGGCCCTGCAAGCTCCCAGCTGATTACCAAGGAGTTTTCGAAGTTGGCCTTGACTGAGG
ATCAAAGGAGGATGGGAGTTCAGGGAATGAGGGTGGGGGTGGGAAATGCCTTAGAATTAA
GTTGAT
ECRD 348 bp, 78% ID
SEQ ID NO: 14
> Human
TTTCCTTCTATCCCTCTGTCTGTCTTACTCTCAGACTATTAATACAAGCCCTGAGTCTGG
CTGTACCCCCAGAACATGTGCCCCGCCCCCTACAACAAAATGCTGCCCCTCCCAGCTAGG
TCTGTTGTTTGTTCCTTTTCTGATTGGCGCCAGGCTTATAGACCCCATGTAGGTAGAATA
TAACTTTCCATAAATAACCTCTAACCCGACCTACAATTTAGCCTTCAGGTTTTTTTCCCC
CTCGTGGTAATGGGATTGCAGCCTGGGCTGATCCATCCTGTATCTTCAGGTCCCAGAAAG
CAGACCCTAGGTTTGGACATTGCTTGGAATTCCTGGTACCCCCATGTT
ECRE 600 bp, 77% ID (human to mouse)
SEQ ID NO: 15
> Human
TGGGGGTTCTGGAAGGAATCCGTGGGAGGCTGGGAGGAAGATCTGGCTTGTCAGCTTCCC
TAGGAAAACCTTCCCCTGGGCTGGCCGCAGGCTGTAACCGGATTCCTGCTCCACCTCTGC
ATCTGGCCCAGGGACCTCATGGCAGGGAGGCCCAGCGCCTGGCCCTTTGCCCCTGGACGG
GGTGGGCCCTGGGTCATGGTGGGGTGGGTGGGGAGGTCAGGAGGGCCATGGGGAGGGGGC
GCGGTGGGGTGCTTTGCCCTGAGAACACAGGCCTCTGGCACCCCGGAGCCCCCGGCAGCT
GCTGGCGTCTGTCAGCCACCTTGCGGGGCGCGGCCGGGGGCTGCTGGCCCCTACATCTTC
CTGACAGGCCCCTCTTCTGAGGCCAGGAAAAAACAACAACAGTTCCTCCCCTCACGGCAA
CCCATTTGTTAGATGAAGGCCGGGCACCAGCACCTTTAACCTCCTCAAAGTCAGCGTTTC
CCTGTCAAGGCCCCACAGGGCCAGAGACAGAGATGGATGGAAGGAGCTGTGTGTCGAAAA
AGCCCTGTGGCCTCATGAGGAGAGCTCTGTTTTCAGGAAGGGAGGGGACCCCGGTTTCTG

One element, ECR5, was able to stimulate transcription in UMR106 cells (FIG. 5B), but not in the kidney cell line, suggesting that ECR5 enhancer function is specific to osteoblastic/osteocytic lineage.

We also tested the transcriptional activity of the human SOST proximal promoter region (2 kb region upstream of 5′UTR) in the two cell types and compared it to the SV40 and the osteoblast-specific osteocalcin promoter (OG2). The SV40 promoter showed comparable activity in both cell lines and, as expected, OG2 was only active in the UMR-106 cells. The SOST promoter demonstrated slightly higher activity than the OG2 promoter in osteoblastic cells, while it demonstrated a threefold stronger activity in kidney cells than the SV40 promoter (FIG. 5B).

These data suggest that SOST kidney expression may be due to proximal promoter sequences, whereas strong expression in osteoblast/osteocyte cells requires the activity of the ECR5 enhancer element. Consistent with the results obtained from transfecting SV40 promoter constructs, only ECR5 was capable of activating the human SOST promoter (4×) in UMR106 cells (FIG. 5C). Thus, a small sequence element within the vbΔ region (ECR5) was identified that confers in vitro osteoblast-specific enhancer activity onto both the human SOST and the SV40 heterologous promoter.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced.

TABLE 1
ECR1 138 bp, 76% ID

| | |
|---|---|
| chicken | ------------------------------------------------------------ |
| opossum | AAAGGAAATGCTCCAGAGAATGACCTCCTCC-----TTTCCCCGTGGTCCAGCT-CTCCT |
| rat | ----AGGA-------G--------CCTCTTG-----GGGACCCATG--CCAGCAACTCCC |
| mouse | ----AGGA-------G--------CCTCTCG-----GG-ACTCATG--CCAGCGACTCCC |
| dog | ----AAGGTGCCACCG--------CGCCCCCTCCGACGTCCCCACG--GCCGCGGCTCCC |
| human | AAGAAAGATGCTGCAG--------CCTCCCC-----AGTCCCCATG--CCAGC-GCTCCC |
| chicken | --------------------------AAGAGCTGAGGACGCAGACAGCAC------GGAA |
| opossum | GCTCGTCTCTGAATTT--TCTGTCTTAAGGAATCCAAGTCGCAGACAGCGCAGAATAGCA |
| rat | AC-TCTCTCCGAACCT--TTG----------------GTGGCAGACAGAGCCCGA-GGCA |
| mouse | AC-TCTCTCCGAGCCT--TTG----------------GTGGCAGACAGAGCCCGA-GGCA |
| dog | ACTTCGCTAGGAGCTCTGTCG----------------GT-----------------GGCG |
| human | ACTTTTCTCTGAGCTT--TCG----------------GTGGCAGACAGCGCCTTG-GGCA |
| chicken | TTGGATCATGGTC---ATTCAAACCACCCGTTTAAGGCAGAGACGTGAAAAAAATA---G |
| opossum | CTTTTTCATTATCATAATTCAAATTACCTGTTTAAGTCGGTCAAATG-AAAAAATAGCCG |
| rat | CGTTTTCATGCTCATAATTCAAATTGCCTGTTGAAGTCGGTCAAATG-AAAAAATANNA- |
| mouse | CGTTTTCATGCTCATAATTCAAATTGCCTGTTGAAGTCGGTCAAATG-AAAAAATA---- |
| dog | CTTTTCCACGATCATAATTCAAATGAGCTGTTGAAG-CGGGTGCATG-AAAAAATA---- |
| human | CTTTTTCATGCTCATAATTCGAATTACCTGTTTAAGTCGGTCAAATG-AAAAAATACCAG |
| chicken | CCCTCAGCCCACACATTGTGGCCTGGCACACAGCAGCGTCC---GAAGATGAATGCAGTT |
| opossum | --TTCAGCCCCCGC-CTGTGGCCTGGCA---GAGGGAACCTTGCGAAGC-GTGTCCTGAT |
| rat | --------------------------------GGGGAGCC----GGAGC-----CCA--- |
| mouse | ------------------------------------------------------------ |
| dog | ------------------------------------------------------------ |
| human | --CTCCGCCCCCAC---GCGGGCTGGCC----GGGGCGCCT---GGAGC-G---CCAGG- |
| chicken | GATGAGATTAATTTGTTGGGCCCTTACTTATCACC-TGCAGAC--------GAGGCTCCG |
| opossum | TCGGAGCTGAAGCGGTCCGGCCCTTCCCTCCCGCC-TGGGCTCACAGACTTCAGGCTCCA |
| rat | -------------------GCGCTGGCCG-CGGCC-TGGGTCC----------------- |
| mouse | ------------------------------------------------------------ |
| dog | ------------------------------------------------------------ |
| human | --GCGGCTGCAGC------GCGCTCTCCG-CGGCCGTCGGCCC----------------- |
| chicken | TGCATCATGAGCTCATTTCCTGGT-G---------------------------------- |
| opossum | CGTGCCATGAGCTCATTTCCTGGGG-GGGTGTGAGTGTGTGTGTATGTGAATGTGAGCTT |
| rat | -------TGAGCTCATTTCCTGGG--------------------------CCGCGCGCCG |
| mouse | -------TGAGCTCATTTCCTGGG--------------------------CCGCGCGCCG |
| dog | -------TGAGCTCATTTCCTGGG------------------------GCGCGCGCGCCG |
| human | -------TGAGCTCATTTCCTGGG------------------------GCGCGCGCGCCG |
| chicken | --CTATTTCAGCCTGGCTCCAGCCAAACAGGGCAATTAGCTGCGGCCAGCAGGGGGTTAA |
| opossum | --CTATTTCAGCCTGGCTCCGTGCAAACAGGGCAATTTACTGTGACCAAAAGGGGCCCAA |
| rat | GGCTATTTCAGCCTGGCGCTGGGCAAACGCGACAATTGACTGCGGCCCACGGGGGCCCAA |
| mouse | GGCTATTTCAGCCTGGCGCTGTGCAAACGCGACAATTGACTGCGGCCCACAGGGGCCCAA |
| dog | GGCTATTTCAGCCTGGCGCTGTGCAAACAGGACAATTTACTGCGGCCAAAAGGCGCCCAA |
| human | GGCTATTTCAGCCTGGCGCTGTGCAAACAGGACAATTTACTGCGGCCAAAAGGGACCCAA |
| chicken | AATGCTCTCCTGGTTCAGCCACACGACCCCCAGGCTCCAGCATGGCCTTC-----TGCTC |
| opossum | ATTACATTCGTAACATAGAAAAATATTCTCCTTGCC------AGGCCACCCATGTGACCG |
| rat | ATTACAATCGGATCCCACACAAATAGCCGCCAGGCT------TGGCCTGC---------- |
| mouse | ATTACAATCGGAGCCCACACAAATAGCCGCCGGGCT------TGGCCTGCCG-------- |
| dog | ATTACAATCGTATCACACACAAATATCCGCCACGCC------AGGCCTCC---------- |
| human | ATTACAATCGTATCACAGACAAATATCCGCCACGCC------AGGTCTCC---------- |

TABLE 2
ECR2 483 bp; 78.7% ID

| | |
|---|---|
| chicken | ------------------------------------------------------------ |
| opossum | ---------------------------------------------------------AAA |
| rat | GGGGTCTCAC--------------------------------AGAATCCTTCAGGCCAGA |
| mouse | GGGATCTCAC--------------------------------AGAATCCCCCAGTCCAGA |
| dog | GGCCTCTTGCTCTTGCCAAGGGGTGAAGCACTGAGGCTCCAGAGAAACCCAAAGTCCGGA |
| human | GGCGTCTTGT--------------------------------------CCCAAGTACAGA |
| chicken | ------------------------------------AAACCAAACACAAACAGCCA---- |
| opossum | GGCCCAGCTCCCTTTACTCTCATGAGCACTGGTCAGAAATTTAAAATAAACAGTTA---- |
| rat | GACCCAGATCTTTTCCCTCTCAGGGGATGTGACCTCAAACTTCAAGCAAATACTTG---- |
| mouse | GACCCAGATCTTTTTCCTCTCAGGGGATGTGATCTCAAATTTCAAGCAAACACCTG---- |
| dog | GCCCTGGGTCCTTTCCCTCTCGTGGACAGTGGTCCAAAATTGAAAACAAAC----ACTTT |
| human | GACCTGGATCCTTTCCCACTCATGTGCAACAGCCCAAAATTAAAAACAAA---------- |
| chicken | --------CACA---ATTTCGCACTT-CACAATGGT---TTTCCATTTTAGAA-CAATGT |
| opossum | -----AAAAACTCTGATATTAAAAAA-G-----------TTCCTTCCTTAAAA-TATTGC |
| rat | AAAATAAAAACC---AAATTAAAAAAACAAAACCAACCTTTCCTCTCTTAAAAATATTGT |
| mouse | AAAATAAAAGCC---AAATTAAAAAA-CAAAACCAACCTTTCCTGCCTTAAAA-TATTGT |
| dog | AAAATAAAAGCC---ATATTAAAAAA-CAAACCCAAC--TTTCTGCCTTAAAA-TATGGT |
| human | --------AGCC---ATATTAAAAAA-CAAAACCAAC--TTTCTGCCTTAAAA-TATTGT |
| chicken | AGGCTAGA-------TCCAGTGAGCCATTACTATTTA-TTTATTACGAACAAGTGGAATT |
| opossum | AA---AGGGAATAGGGATAATTAGCAATTATCCTGCACTTTATTATGAGAAGACAGAATT |
| rat | GAGCCAGGGA-----AGCAATCTGCCTCAATAGTGCATTTTATTATG---AGATAGAATC |
| mouse | GAGCCAGGGA-----AGCAATCTGCCTTGACGCTGCGTTTTATTGTG---AGGTAGAATT |
| dog | GAGCCAGGG------GGCAATTAGCAATTATGCTGTATTTTATTATGAGAAGATGGAATT |
| human | GAGCCAGGG------GGCAATTAGCAATTATGCTGTATTTTATTATGAGAAGATAGAATT |
| chicken | CAAATTAGGCTGATTCGAGCCTCAGCAACCCCCTCAGATTGGATTGGTAATTAAGGGAC- |
| opossum | CTAATTAGACTGATTTGAATTCCACACACCTCCACAGATTGTTTTGGGAATTAAGGTATC |
| rat | CAAACAGGACTGGTTTGGGCTCTGAACACCTCCACACATTGTTTGGGGAATTAAGGGATC |
| mouse | CTAACTGGACTGGCTTGGGTTCGGTACACCTCCACACATTGTTTGGGGAATTGAGGGATC |
| dog | CTAATTGGACTGATTTGAATTCCACACACCTCCACAGATTGTTTTGGGAATTAAGGTATC |
| human | CTAATTGGACTGATTTGAATTCCACACACCTCCACAGATTGTTTTGGGAATTAAGGTATC |
| chicken | --TCGTACTGGTAATTATGGTTTACCATGAAATTACCCTCCCTTCTCCCCTCTCCCCCCA |
| opossum | AGTTGTATGGGTAATTATGGTCTACCGTTAZATTGCC-----------GCCCCCCCAAAA |
| rat | AGTCGTATTGGTAATTATGGTTTAGCATTCAATTACC------------CCTCCAGTTAA |
| mouse | AGTAGTATTGGTAATTATGGTTTAGCATTCAATTACC------------CCCACAGTTGA |
| dog | AGTTGTATCGGTAATTATGGTTTACCATTCAATTACT------------CCCCCCACAGA |
| human | AGTTGTATCGGTAATTATGGTTTACCATTCAATTAC-------------CCCCCCACAGA |
| chicken | AAAACACCAAATTAGCCACGAGTGGAATTATATTTATCACAGCAGCCCGTGCAGGAGGGA |
| opossum | AAACTGTTAAATTGTCTGTGAC-GGGCTTAAATTTAGCCCAGACTCATGTCCAATGAAGG |
| rat | AAACCGTTAAATTGTCTGTGACAGGGCTTAAATTTAGCCCAGTCTCATGTCCTATGAAGA |
| mouse | AAACCGTTAAATTGTCTGTGACAGGGCTTAAATTTAGCCCAGTCTCATGTCCTGTGAAGA |
| dog | AAACTGGTAAATTGTCTGTGACGGTGCTTAAATTTAGCCCAGACCCATGTCCTATGAAGA |
| human | AAACTGTTAAATTGTCTGTGACGGGGCTTAAATTTAGCTCAGACCTATGTCCTATGAAGA |
| chicken | CT--TTGTGAGTAAATACAAACAAGGAGGAAACCA--CTG----CGCCGCGCCAGCGTGA |
| opossum | CT--GGGCAAGTCAATCTAAACAGCCAGGAAACCACTCTAGAA-CCTGGTGCCAA-GCAG |
| rat | CT--TCACGAGTCAATACAAGCCATCCGGAAACCA--CAAGGT-CCCTGTGCCAG-GTGG |
| mouse | CT--TCACGAGTCAATACAAGCCATCCGGAAACCA--CAAGGT-CCCTGTGCCAG-GTGG |
| dog | CTGCGCGCGAATCAACACGAGCCGCCCGGAAACCA--CTGGGTGCCCTGTGCCAG-GCAG |
| human | CT--GCGCGAGTCAATACAAGCCATCCGGAAACCA--CCGGGTGCCCTGTGCCAG-GCGG |
| chicken | TAATTA-----GGGTTTGAAGGC-AGGAATTCTTGCAGGAGCCA---------------- |
| opossum | TAATTA-----GGGCCTGAGGTGAAGAAAGTTTTACCTGAAACAGCAG------TAATCA |
| rat | TAATGACCAGTTGGGATGAGGTT-------TTTTATCTGGGACATCGGGACAAGGGACAA |
| mouse | TAATGACCAGTTGGGTTGAGGTT-------TTTTATCTGGGACATCGGGACAAGGGACAA |
| dog | TAATTA-----GGGGTTGAGGTTTCCAAAATTTTACCTGAGACAGC------AGGGACAA |
| human | TAATTA-----GGGGTTGAGGTTTCCAAAGTTTTACCTGAGACAGC------AGGGACAA |
| chicken | --------------------------------- |
| opossum | GCTCACTGGGGAG-GGAGT-------------- |
| rat | G----CTGACCTACAGGGCATTTATGTTGGGGG |
| mouse | G----CTGACCTACAGGGCACTTGTGTTGGGGG |
| dog | GCCC-CTGGGCTGCAGGATGCTCACGT-GGAGG |
| human | GTGC-CTGGGCTG-GGCGTGCTCACGTGGGGGG |

TABLE 3
ECR3 360 bp; 78.1% ID

| | |
|---|---|
| opossum | GCAGG---------------TCATGAGTC--ATTTCCCAGCATTCTGGTGGTCCCAGCAA |
| rat | GCATG-CTCAGCCCCGCTC-CCCTGAGACCAGGTTCCCATCACTGGGGTGGCTGGGGCAG |
| mouse | GTCTG-CCAAGCCCTGCTC-CCCTGAGACCAGGTTCCCATCACTGGGGTGGCCTGGGCAG |
| dog | GCAAG-CCAAGTCCTGTTGGACCTGGACCCAGATTCCCATCTGAGGGGTGGTCTGGGCAG |
| human | GTAGGGCCAAGCCCTGTTCAGCCTGGGACCAAGTTCCCATCAACAAGGTGGTCTGGGCAG |
| opossum | TTACAGAGCA-AGTG-AGTAATTAAGC-CCAAAGATCGAGG---CAAGGG--GAA-CCCC |
| rat | CTGCCAGCCA-AGAGTCTTAATTACTA-TAGGCTTGCAGGGATCTCGGCT--AGTTCCCC |
| mouse | CTGCCAGCCA-ACAGCGTTAATTACTA-CGGACTTGCAGGGAGCTCAGCG--AGC-CCCC |
| dog | TGGCCAGCCAAAAAGTGGTAATTACTTCTCGAGGTGTGGGGAGCTCAGGT--GGC-CCCC |
| human | TGGCCAGCCAGAAAGCAGTAATTACTG-TCGAGGTGCAGGGACCCCAGGTAGGGC-CCCC |
| opossum | GC---------------------ATGAGGCATGAATGAAAACT-CCGAGCTGGTAAACTG |
| rat | ACCCCCTACATTCCCATTTCTAAGTGGGAACTGGATGGGCCCA-CCCTG--GGTAAAAGG |
| mouse | ACCCCCTACATTCCCATTTCTAAGTGGGAACTGAACGGGCCCA-CCATG--GGTAAAAGG |
| dog | AG-------GCCCTCACCTCCATGTGGGCAGTGAATGG------CCCTG--GAAGAGATG |
| human | AC--------CTCCCACCTCTGTGTGGGCAGTGAATGGGCCTGCCCCTG--GGTAAGGCT |
| opossum | GTGTGTCAGCAGCCACCC-CTCACCCCTTACTGTGTTCCCAGGGCCCTTGAGCTGTCTAC |
| rat | G--TGTCAGCAGGCACCTGTTCACCTCTTGCTGGGTTCCCAGGCCCCTAGAGCCC-CTTG |
| mouse | G-GTGTGAGCAGGCACCTGTTCATCCCTTGCTGGGTTCCCAGGCCCCTAGAGCTC-CTGG |
| dog | G--TGTCAGCAGGCGCCTGCCCACCCCTTGCTGGGTTCCCAGGCCCCTGGAGCCCTCTCA |
| human | G--TGTCAGCAGGCGCCTGCCCACCCCTTGCTGGGTTCCCAGGCCCCTAGAGCCCTCTCG |
| opossum | TTCAATGATGCACTTGCCCTGTAACCAGTGGGTGATCAGATTTTTAATCTCATAGATCTC |
| rat | GAATAGTGACCGTTTGCCCTGCAAGCAGTGGGTGACCAGGTTTTTAATCTTGGAGACCCC |
| mouse | TAATAGTGACCGGTTGCCCTGTAAGCAGTGGGTGAGCAGGTTTTTAATCTTGGAGACCCC |
| dog | TAATAGGAGCCATTTGCCCCATAACCAGTGGGTGACCAAGTTTTTAATCTTAGAGGCCCC |
| human | TAATAGGAGCCATTTGCGCTGTAACCAGTGGGTGACCAGATTTTTAATCTTGGAGACCCC |
| opossum | TAATAACCCAGTGAAGGGGGGAGAGGCAGGCAGGTTTTGTCAAATAGGAGAAGAGGAAGG |
| rat | TCAGATACCAGCT-----------GGGAAGTGGGATATGTCAAAATGGGAGGGAGGGAGG |
| mouse | TCAGATACCAGCT-----------GGGAAGTGGGATTTGTCAAAACGGGAGGGAGG---- |
| dog | TTGGATCTCAGCT-----------GGGAAGTGGGATTTGTCAAATGGGGAGGGGAGG--- |
| human | TTGGATCCCAGGC-----------GGGAAGTGGGATTTGTCAAATGGGGAGAGGCGG--- |
| opossum | AG------TCCTGGTTGTGAATGTGGGA |
| rat | GAGAGAGAGCCTGTCTGTGAATGTCAGA |
| mouse | --------TCCTGTCTGTGAATGTCAGA |
| dog | --------GCCTGTCTGGGAACGCCAGA |
| human | --------GGCTGTCTGGGAATGCCAGA |

TABLE 4
ECR4 239 bp; 75.7%

| | |
|---|---|
| opossum | CTGTCCTAAG----CTGTCTTCTC------TCCTGGAGAGGTCC-AAGTTTTTGTTTACC |
| rat | CT-------GTATCCTGTCCCCTA------CCC----AAGGACC-AGGGCTTTGTTTACT |
| mouse | CT-------GCATCCTATCCCCTATCCTAACCC----AAGGGCC-AGGGCTTTGTTCATT |
| dog | CTCTC--AAGCATCCCTGCCCCCA------CTC----AAGGGCCCAGGGTTTTGTTTACT |
| human | CTCTC--AAGCACCCCCTCCCCCA------CCC----AAGGACC-AGGTCTTTGTTTACT |
| opossum | AA-AC---TCAAAGATGCACTTTCTCTTCCTGATTTCATCAATTATAAATGTGCTTGCTA |
| rat | AA-ACATCTTGGG-ACGAGTTCTCACC--------------------------------- |
| mouse | GAGACATCTTGGG-ATCAGTTCTCAACCCCTGATTTCATCGATTATAAATGTGCTTGTTT |
| dog | AA-ACA--------ATGAGCTCTCACCCCCTGATTTCATCAATTATAAATGTGCTCGCTA |
| human | GA-GCATCTCAGCGATGAGCTCTCACCCCCTGATTTCATCAATTATAAATGTGCTCGCTA |
| opossum | CTCACCACTGTGCAATTTGTGAAGACCTGTGGCTTGTGGTAAGAGTGGCTACATCCAGCC |
| rat | ------GCAAGGCAATTTGTG-TTGACCGTGGTCTGTGGTGACACTAACACCTTCCAAGC |
| mouse | CTCACGGCAAGGCAATTTGTG-TTGACCGTGGTCTGTGGTGACACTAACACCTTCCGGGC |
| dog | CTCACCACAGGGCAATTTGTGACGGACTGTGGTTTGTGGTGAGAGTAGCACCATCCAAGT |
| human | CTCACCACACGGCAATTTGTGACGGACTGTGGTTTGTGGTGAGAGTAGCACCATCCAAGT |
| opossum | TTACCACAGCGGT--GAAGAGATGGGGTGTTGGGGCCAGACAAATGGAGGTGGTGGTGGC |
| rat | TCACCACAGCCTTG-GCAGGG--GACGGGGTGGGGGCAGATACAGAGCTGTGGGGGCGGC |
| mouse | TCACCACAGCCTCG-GCAGAGAGGAGGGAGGAGGGCCAGATACAGGGCTGTGGAGGTGGC |
| dog | TCACCGCAGCTGTGAGTAGAGACGA-GGGTTAGGGCCAGACACAGGGCTGTGGGGGCGGC |
| human | TCACCGCAGCCGCGAGTAGAGATGA-GGGTTGGGGCCAGACACAGGGCTGTGGGGGCGGC |

TABLE 5
ECR5 268 bp; 79.1% ID

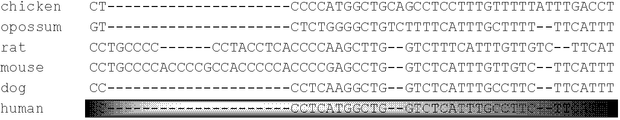

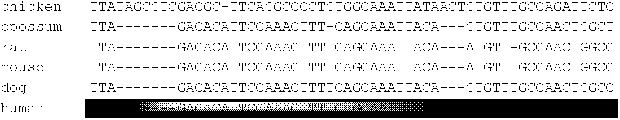

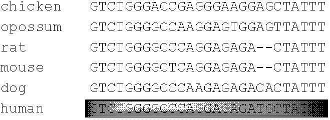

TABLE 6
ECR6 468 bp; 76.9% ID

| | |
|---|---|
| opossum | ------------------------------------------------------------ |
| rat | TTAG-------CCAAGTGGTACGGGTTCTCTGTGTATGTGG-----------AGTGTATA |
| mouse | TTAG-------CCAAGTGGCGCGGGTTCCCTGTGTATGTGG-----------AGTGTGTA |
| dog | TCAGAGCCTGGCAAAATCTTACTG-----TGGCGCGTGCTGCAGGCTGTGCATGTGTATG |
| human | TCAGGGCCTCTCAGAGTCCTGCCG-----TGGTGTGCACTG-----------TGTGTGTG |
| opossum | TTTTCCCTTCTA---TTTGGATGGGATCCTAATCCAATAAATGCTTTGGAGATTTCTATG |
| rat | TTTAATCTTCTATATTTTGGATGAGTTCCTAATCCAATAAATGCTTAGGAAATTTCTATA |
| mouse | TTTAATCTTCTATATTTTGGATGAGTTCCTAATCCAATAAATGCTTAGGAAATTTCTATA |
| dog | TTTAATTCTCTACA-TTTGGATATGGTCCTAATCCAATAAATGCTTAAGAGATTTCTCTA |
| human | TTTAATTTTCTACA-TTTGGATGTGATCCTAATCCAATAAATGCTTAGGAGACTTCTATA |
| opossum | GAATAGATTAATTTTTACTAGAAAAAAACATAATTGGTAGATGTTAAGGCTATTGCCCTG |
| rat | GAACAGATTAATTTTTACTAGAAAAAAATATAATTGGCCGATGTTAAGGCTACTGCCCTG |
| mouse | GAACAGATTAATTTTTACTAGAAAAAAATATAATTGGCTGATGTTAAGGCTACTGCCCTG |
| dog | GAATAGATTAATTTTTACAAGAAAAAAATATAATTGGTTGATGTTAAGGCTACTGCCCTG |
| human | GAATAGATTAATTTTTACTAGAAAAAAATATAATTGGCTGATGTTAAGGCTACTGCCCTG |
| opossum | ACAAAT-CTGCCTCGGCTATATTTTTGAAAAAGTAAAAGACCTGATAkACTTCCCCATAA |
| rat | ACAGATCCTGCCTTGGCCATCTATTTGAGGAAGCGAGAGGCCTGATCGGCGCGCTCATAA |
| mouse | ACAGATCCTGCCTTGGCTATCTATCTGAGAAAGCGAGAGGC------------CTCATAA |
| dog | ACAAAT-CTGCCTTGGCCATATATCTGAGAAAGTAAAAGACCCCATAGACTCACACATAA |
| human | ACAAAT-CTGCCTTGGCCATATATCTGAGAAGGTAAAAGACCCGCTACGCTTGCACATAA |
| opossum | ATATGCCATCTGTTCAGCGAGACCTGGGCGAGGAGCTCCCAGGAGGTTTCCCTGGGTGAT |
| rat | ATATGCCATCTTCCCG-TGGGCCCTG---AGAGCACTCCTCGGAGGCTTCCCGCCGTGAT |
| mouse | ATATGCCATCTTCCCC-TGGGCCCTG---AGAGCACTCCTCAGGGGC-TCCTGCCGTGAT |
| dog | ATATGCCATCCTCCCCACAGGCCCTG-GTGAAGCACTCCGGGGAGGGTTCCCTCTGTGAT |
| human | ATATGCCATCTTCCCCACAGGCCCTG-GAGAAGCACCCCGGGGAGGTTTCCCTTGGTGAT |
| opossum | TTATTCTTCATTAATAAGCTCTATTCTATATTAGTATCAGATTTATGGTTCTGCCTTTCG |
| rat | TTATTCTTCATTAATAAGCTCTATGCTGTATTAGGATCAGATTTACGACTCTGCCTCTCT |
| mouse | TTATTCTTCATTAATAAGCTCTATGCTGTATTAGGATCAGATTTACGACTCTGCCTCACT |
| dog | TTATTCTTCATTAATAAGCTCTATGCTATATTAGGATCAGATTTATGACTCTGCCTTTCC |
| human | TTATTCTTCATTAATAAGCTCTATGCTATATTAGGATCAGATTTATGACTCTGCCTTTCT |
| opossum | AAGATTTGGGGGCATTTTATCTGAGTAGAATTTCAAATGAGTGCTTGAAACTTCG-CAGC |
| rat | AATATTT-CTGACATTTCATCTGAAAGGAATTCCAAAAGGAATTGTGAAACGTTGCCCAC |
| mouse | AATATTT-CTGACATTTCATCTGAAAAGAATTCCAAACGAAATCGTGAGACATTA-CCAC |
| dog | AATATTT-CTGACATTTCATCTGCAAAGAATTACAAATGGTATCTTGAAACTTTG-CTGC |
| human | AATATTT-CTGACATTTCATCTGAAAAGAATTACAAATGAAATCTTGAAACTTTG-CCAC |
| opossum | TTTTCTCTCCGCAGGC-------------------------------------------- |
| rat | ATCT-TCCCTGCCAGTGTTCCAAAACCTCACATTTAAGGGG-GCTGCCGGTTTGGGGTGG |
| mouse | GTCT-TCCCTGCTAGCGTTCCAATACTGCACACCTAAGGTT-GCTGCTGGGATGAGGTGG |
| dog | TTC--TCCTGGCTGGTGCCTGGGCACTGTCCGGCCAAGGGGAATCAGCGGGATGGAGAGG |
| human | TTC--TCCCTGCTAGTGCTCTGGCACTCTGTGTCCAAGGGGAGATGGTGGGCTGGGGAGA |

TABLE 7
ECR7 581 bp; 88.1% ID

| | |
|---|---|
| chicken | ------------------------------------------------------------ |
| opossum | TCTCTTGATAAAACCTCAGATCTCCATTTTTCTTGAGCTAGTGTAGACAAAGCTGGTTAG |
| rat | TTCC------AAGGATTAGAAGTTC-----CCTCTTGTT--------------------- |
| mouse | TCCC------AAGGATAAGAAATTC---TCCCTCTTGTT--------------------- |
| dog | TTCA------AAGGGTTAGAACTCA---TTTCTCGACAT--------------------- |
| human | TTCA------AAGGGTTAGATCTCA---TTTCTCTGCCT--------------------- |
| chicken | ------------------------------------------------------------ |
| opossum | TAAGTTTTCACCTCTATCTA--GGTTCTCAGAAGTGTGCTGGCCTC----------CCCC |
| rat | ----TCTTCACTTGGGCCCA--GAG-CTCAGAATTGGGTGGACTCC------TCCACCTC |
| mouse | ----CCTTCACTTGGGCCCACAGAG-CTCAGAGTTGAGTGGATCCC------TCCACCTC |
| dog | ----TTCCAGCGTGGGCCCA--GAG-CTCAGAACCTTGTGGGCTTCTTTCTCCCTGCTCC |
| human | ----TTCTAGCTTGGGCCCA--GGG-CTCAGAAATGTGTGGACTCC---CTCACAGCCCC |
| chicken | ------------------------------------------------------------ |
| opossum | CACCAAAAAAATCCCCCCATCCCAT---TTTCAGACATGTGACACCTGTATTATTGCTAA |
| rat | TCCCAGGGTCCCTGCCCCCTCCCAACTGCCACAGGCAGGTGACACCTGTATTATTGCTAA |
| mouse | GCCCAG--CCCCTGCCCCCTCCCAACTGCCACAGGCAGGTGACACCTGTATTATTGCTAA |
| dog | TCCCA-CATCCCCGCCCCCTCCCAG------------GGTGACACCTGTATTATTGCTAA |
| human | TCCCAGCATCCCTGCCCCCTCCCAACTGCCTTGGGCAGGTGACACCTGTATTATTGCTAA |
| chicken | --------------------CAATGGAGTCCATCACCCAATG---GCTTCTGGAAGGCGA |
| opossum | GGGTTAGAAAGCTCACGAATCAATAAAACCCATTAATGAGTGTTGGTACCTCGAAGGCTA |
| rat | GGGTTAAAAAGCCTTCAAATCAATAAAACCCGTTAATGAGTGTTGGTACCTCGAAGGCTA |
| mouse | GGGTTAAAAAGCCTCCAAATCAATAAAACCCGTTAATGAGTGTTGGTACCTCGAAGGCTA |
| dog | GGGTTAAAAAGCCCCCGAATCAATAAAACCCATTAATGAGTGTTGGTACCTCGAAGGCTA |
| human | GGGTTAAAAAGCCCCCAAATCAATAAAACCCATTAATGAGTGTTGGTACCTCGAAGGCTA |
| chicken | TGGGTAAATCCCTTCAATC-----AACTCCACGCCACAATGCATCTCTTTTCTTCTTTTT |
| opossum | CAGATAAATCCGTTCTACTCAGTGAGTTCAATCCCATAAAACAGCTCTC----------- |
| rat | CAGATAAATCCCCTCTATCCCGTGAGTTCAGTCCCATAAAACAGCGCTC----------- |
| mouse | CAGATAAATCCCCTCTATCCTGAGAGTTCAGTCCCATAAAACAGGGCTC----------- |
| dog | CAGATAAACCCCTTCTACTCAGTGAGTTCAATCCCATAAAACAGCTCTC----------- |
| human | CAGATAAATCCCTTCTACTCAGTGAGTTCAATCCCATAAAACAGCTCTC----------- |
| chicken | TCCCCTTTAATTATGGAGTTGGTTTAACAGCAAATGAGAAGAAATGTTTAAAAGGTGACT |
| opossum | -TCCTTTCCATTCGAGCCTTCATTTGATAGAAAATGTGGAGAAATTCTAAAAAGGTGACT |
| rat | -CCCTCTCCATTGCGCGATCCATCTGACAGAAAATGTGGAGAAATTTTTAAAAGGTGACT |
| mouse | -CCCTCTCCATCCCAGCATCCATCTGACAGAAAATGTGAAGAAATTTTTAAAAGGTGACT |
| dog | -CCCTTTCGATCCTGGCATTCATTTGATAGAAAATGTGGAGAAATTTTTAAAAGGTGACT |
| human | -CCCTTTCAATCCTAGCATTCATTTGATAGAAAATGTGGAGAAATTTTAAAAAGGTGACT |
| chicken | TATTAATTGCCTGTAAATTAAAAAGCAGACGGATGCTTTATTGCAGTTGAGAGAAGTTAG |
| opossum | TACTAATTGCCTGTAAAATAAAAGGCAGATGGAAGCTTTATTACAGTTGAAGGAAGTCGG |
| rat | TACTAATTGCCTGTAAAATAAAAGGCAGATGGAAGCTTTATTACAGTCGAAGGAAGTCAG |
| mouse | TACTAATTGCCTGTAAAATAAAAGGCAGATGGAAGCTTTATTACAGTCGAAGGAAGTCAG |
| dog | TACTAATTGCCTGTAAAATAAAAGGCAGATGGAAGCTTTATTACAGTTGAAGGAAGTCGG |
| human | TACTAATTGCCTGTAAAATAAAAGGCAGATGGAAGCTTTATTACAGTTGAAGGAAGTCGG |
| chicken | TAATAGTAAGGTATAATGTCAAATGGCAATTGACTTCTTTAGGGTTCTCTTTTCTTTTCT |
| opossum | TAATATTAAGGTAAAATGTCAAATAACAATTGATTT--------------------TCTC |
| rat | GAATATTAAGGTAAAACGTCAAATAACAATTGATTT--------------------TCCT |
| mouse | GAATATTAAGGTAAAATGTCAAATAACAATTGATTT--------------------TCCT |
| dog | GAATATTAAGGTAAAATGTCAAATAACAATTGATTT--------------------TCCT |
| human | GAATATTAAGGTAAAATGTCAAATAACAATTGATTT--------------------TCCT |
| chicken | AACACATAAAGGAGTGATTTATCGCTTCCTGCAGACTG--AGGGATAAATCATCCTGGAA |
| opossum | CAGACATAAAGATGTGATTTATGGCTTCCTAGTTACTACAAACGAGAAATTATT-TGAAG |
| rat | TAGACATAAAGGGACGATTTATGGCTTCCTAGTTAGTACAAATGAGAAATTATT-TGAAG |
| mouse | TAGACATAAAGGGACGATTTATGGCTTCCTAGTTAGTACAAATGAGAAATTATT-TGAAG |
| dog | CAGACATAAAGGGGCGATTTATGGCTTCCTAGTTACTACAAATGAGAAATTATT-TGAAG |
| human | TAGACATAAAGGGGCGATTTATGGCTTCCTAGTTACTACAAACGAGAAATTATT-TGAAG |
| chicken | GTCTGAAAA--CTGGGGCTAAATAAAGAT------------------------------- |
| opossum | TTCTAAAAAGTATGAGGTGAAATAAAGATTAAATAGAAGATGAAATCATAGAAC------ |
| rat | TTCTAAAAAGTATGAGGAGAAATAAAGATTAAATAGAAGATGAAATCATAGAGGTTTTTT |
| mouse | TTCTAAAAAGTATGAGGAGAAATAAAGATTAAATAGAAGATGAAATCATAGAGTTTTTTT |
| dog | TTCTAAAAAGTATGAGAAGAAATAAAGATTAAATAGAAGATGAAATCATAGGGA------ |
| human | TTCTGAAAAGTATGAGGAGAAATAAAGATTAAATAGAAGATGAAATCATAGGGA------ |
| chicken | ------------------------------------------------------------ |
| opossum | ------TTTCTCTGGGAGGTGACTTAAATGCCCCTGGGAGTGAGGATTCATTCAGTCA-G |
| rat | TTCCCCCTCCCCTGGGAGGTGACTTCAGTGTCCCCAGAGACTAGAATTCATGTAGTTG-G |
| mouse | TT----TTTTTCTGGGAGGTGACTTCAGTGTCCCCAGAGACTAGAATTCATGTGACTGAG |
| dog | ------TTTCTCTGGGAGGTGACCTCAATGCCCCTGGGGGCTTGAATTCATGTGGCGG-- |
| human | ------TTTCTCTGGGAGGTGACTTCAGTGCCCCTGGGGACTAGAATTCATGTGGCCA- |
| chicken | ------------------------------------------------------------ |
| opossum | GTCCTGAG-----------------ACAGCTGAGGCAGGGGGATGACTAGGACTAGATTT |
| rat | GGTTGGGG---------------GGTAAGCCAAGCCAG--------ATGGGGCTTGGCTT |
| mouse | GGGTGGGGGAGGTTGTGGGGAGAGGTAAGTTAAGACAG--------ATGGGGCTTGGCTT |
| dog | -----AGG--------------------GCCTAGGCAG--------CTGGGGCTTGGTGT |
| human | -----GTG--------------------GCCTAGCCAG--------CTGGGGCT-----T |
| chicken | --------------------------------------- |
| opossum | AGACATGTCC----------CCTGGGGGCTTTGCAGTAG |
| rat | AGCCA-GTCAAGATTCA---CCTTGGGGTATCCAAGGAG |
| mouse | GGCCA-GTCAAAATTCA---CCCTGGGGTATCCAAGGAT |
| dog | GGCAGCTTCAAGATTTAGAGGCGAGGTGTCTCTAAGGGG |
| human | GGCAGTTTCAAGATTTAGAGGCAAGGTGTCTCTGAGGAG |

TABLE 8
ECR8 276 bp; 75% ID

| | |
|---|---|
| rat | GGCTATTTCTAGGCAAAACCGCCTGATGAGATGAAATAGCTCAATTGCTGACCATTAAGT |
| mouse | GGCCATTTCCAGGCAAAAGCACCCGATGAGATGAAATAGCTCAATTGCTGACCATTAAGT |
| dog | AGCTATTTCTAGGCAGAACCATAAGATGAGATAAAATAGCTCAACTCCTGACCATTAAGT |
| human | GGCTATTTCTAGGCAAAACTGTCTGATAAGATAAAATAGCTCAACTCCTGACCATTAAGT |
| rat | CACGAAGGCCATGGCCGTTGTAAATCTCCATCTCGCCATAG--CCTTGGCCTGCAA-GGA |
| mouse | CATGAAGGCCATGGCCGTTGTAAATCTCCAACTCAGCACAG--CTTTGGCCTGCAA-GGA |
| dog | CATGAAGGCCATGGCCATCGTAAATCTCAGTCTCTGCGGCCCTCTCTGTCCTGCGTGGAA |
| human | CGTGAAGGCCATGGCCATCGTAAATCTCA-TCTTTCCGGCC--CTCTGGCCTGCAT-GCA |
| rat | GTGCTGGGTACTGCCA----GGACCAGCCACCTTTGTGGGAAGAGC |
| mouse | GTGCTCGGCACTGCCA----GGACCAGCCACCTCTGTGGGAAGAGC |
| dog | GCAC--AGCCCAGCCA----GTGCCAGCTGCTTTGAT-----GGGC |
| human | GTGC--AGCCCAGCCAGTCGGTGGCAGCCACCTTGGTAGGAAGGGC |

TABLE 9
ECR9 300 bp, 70% ID

| | |
|---|---|
| opossum | CCCACTGTGTTTATGACTGAA--AATGCTGCAGCTTTTTAAAAAGTGTGTTAATTTCTTA |
| rat | C--GTGGGGTTTATGGTTTCATGAGTGTCGGAGTGAT-AAAAA--TGTGGTTATTTCC-- |
| mouse | C--GTGGGGTTTATGGTTTCATGAGTGTCAGAGTGATAAAAAA--TGTTGTTATTTCC-A |
| dog | C-CATGAGGTTTATGGTTTCATGAATGTCGAAGCATTTTAAAA--TGTGGTAATTTCG-A |
| human | C-TGTGAGGTTTATAGTTTCATGACTGTCAGAGCTTTTTAAAA--TGTGGTAATTTTA-A |
| opossum | GAGTTCTGCCCCAGCAATTCCCAGGGTTTCTTTTTCTTTAATTGAATAAATAAATAATAC |
| rat | ------AGCC--------TCCCAGAGTCTGTCTTGCTCTAATTGAAGAAATAAACAATCT |
| mouse | GCCTGTAGCC--------TCCCAGAGTCTGTCTTGTTCTAATTGAAGAAATAAACAATCT |
| dog | GTGTGCAGCC--------TCCCAGGGCCTCTCTCCTTTTAATTGAAGAAATAAACAATCC |
| human | GTGTGCAGCC--------TCCCAGGGTCTTTCTTCTTTTAATTGAAGAAATAAACCATCT |
| opossum | A---TGAGGCCTACT-GGCCAAGGAGAAAATCAGGTGCTGAGCTTCCT--G |
| rat | ACCCTAAGGCATGGTTAGCCTAGGACAGAAGTGTGTGGGAGATCTGCTGGG |
| mouse | ACCCTAAGGCACGGTTAGCCTAGGACAGAAGCGTGTGCGATATCTGCT--G |
| dog | CCCCTAAGGCATGCTCGGCCACGGAGAAAGGCAGGTGCAGTGTTCA-G--G |
| human | CCCCTAAGGCATGCTTGGCGAAGGAGAAAGGCAGGTGCAAGGCTCACA--G |

TABLE 10
ECR10 287 bp, 76% ID (human to mouse)

| | |
|---|---|
| opossum | ---GCCTGTGTTTTCCAAACCAT-TTGCCTGGGAGCCAACAGTGGACCTATTT------- |
| rat | GG-CCCC---CC--------CTC-TTTCTTTCCAGGTAGCAGGACCATTCTGT------- |
| mouse | GC-CCCC---CTTTCCAA--CTC-TTTCATTCCAAGCAGCAGGGCCATTATGT------- |
| dog | GT-GCAC---TTTTCCAG--CAC-TTGCCTATGAGGCAGCAGTGCCATTATTTTTTTGGT |
| human | GCCTCCC---TTTTCCAG--CCCCTTGCTTTCGAGGCAGCAGTGCCATTATTT------- |
| opossum | -GGGAAAGTGGCCTAACAAGATAGGACAGGAACTGGCAACCCAGGGATTTATGTGGTGTG |
| rat | -GGGGGAAACAGCTAAGCAGATAGGACA-------GCAAGCTGGCGATTTATGTGGTGTG |
| mouse | -GGAGGAAACAGCTAAGCAGATAGGACA-------GCAAGCCCGCGATTTATGTGGTGTG |
| dog | GGGGGGGACCAGCTAACCAGATAGGACA-------GCAAACTGGGGATTTATGTGGTGTG |
| human | -GGGGAAACCAGCTAACCAGATAGGACA-------GCAAACCGGGGATTTATGTGGTGTG |
| opossum | GGAATGGCTCACATTTCCGTCACTGTTTACACGGCAGTA---TTTTTAAAAAAGAAATAA |
| rat | GGAACGGCTCCGGTTTCCCTCACTGTTTACCCAGCAGTATTTTTTTTAAAACAGAAATCA |
| mouse | GGAACGGCTCCGCTTTCCCTCACTGTTTACCCAGCAGTA--TATTTTAAAACAGAAATCA |
| dog | GGAAGAGCTCGGATTTCCCTCACTGTTTATCCAGCAGTA--TTTTTTAAAACAGAAATCA |
| human | GGAACAGCTCAGGTTTCCCTCCCTGTTTACCCAGCAGTA--TTTTTTAAAACAGAAATCA |
| opossum | ATGCTAGTGAAAACCACAGCTGTGAATTACTTATAAGGGGCTATGAAAGGGGGTGGGGAG |
| rat | GCATGCA-GAGAACCGCAGCTGTGAATTAT-------GGGCTCT------GGCTGGGAGG |
| mouse | GCATGTG-GAGAACCGCAGCTGTGAATTAT-------GGGCTCT------GGCTGGGAGG |
| dog | GCGCGAG-GGTAACCACAGCTGTGAGTTAC-------TGGCTCT------GGCTGTGAGG |
| human | GCGTGTG-GGTAACCGCAGCTGTGAGTTAC-------TAGCTCT------GGCTGTGAGG |
| opossum | GTAGGAGTATTCCCTATAGAGCAGAGTTCC-------------------------CTTTT |
| rat | GTC----------------ACAAGGGTCCC-------------------------TCTTT |
| mouse | GTC----------------ATGAGGGCACC-------------------------TCTTT |
| dog | GCT-------------------------GTNNNNNNNNNNNNNNNNNNNNNNNNNTCCTT |
| human | GCTGG-------------GGTGGGGGGAGT-------------------------CTCTT |
| opossum | CACCAATCTGAATGCC |
| rat | CA-------GAGTGCT |
| mouse | CA-------GAGTGCT |
| dog | CA-------TAGTGCT |
| human | CA-------GAGCCCT |

TABLE 11
ECPA 666 bp, 75%

| | |
|---|---|
| opossum | ------------------------------------------------------------ |
| rat | TCTTTTTCCTGAAGGGAGGGCCAGATGACTCC-TGAAGAGCTTTTGTTATCCCAGGTATG |
| mouse | TCTTTT-CCTGAAGGGAGGGCTAGATGACTCCCTGAAGAGCCTTCCCTATCCCAGGTATG |
| dog | TATTTT-AGCAAAGTCTGGACTAAATGATGCCTTG--GGGCCCTCATCGCCCCAGGTTTC |
| human | TCTTTC-AGTAAAGTCCAGACCAAATGATTCTTCA--GGGTCCTTGTTCCCCCAGGTTTC |
| opossum | ------------------------------------------------------------ |
| rat | GATGACATT-TTAAAGAGTCACCCTG---ACACCATCATCAAACGTT-GGCAGTCTCCTT |
| mouse | GATGACATT-TTAAAGAGTCACCCGGCGCACACCATCATCAAAAGTT-GGCAACTTCGTT |
| dog | GAGGACATTCTTCCTGGTTCCCT---------CCATCACAACAGGCTCTGGAGTCTGTTT |
| human | AATGACACT-TTAAAGGTGTT-------------GTCGTAAAAGGCTGGGGAGTCTGTTT |
| opossum | ------------------------------------------------------------ |
| rat | TCAC-CTCCAGGTCTCCTTTCCGAAG---AAAAGC-ACTGGGCTGGCTGGCAGGGGGTGT |
| mouse | TTAC-CTCCGGGTCTCCTTTCCGAAAGGAAAAGGC-ACCGGG---GCTGGCAGAGAGCAT |
| dog | CCAT-CTCCAGCTCTCTCCTGAGAAG---AGGGGCCACTCGG---GCTCTCTTGAAACAA |
| human | CTATGCCCCCAATCTCATTTAAGAAG---GGGGGC-ATCCAG---GCCATCATGGAACAG |
| opossum | ----CCCAGAAAACCCTCCTTTCTCTCCAACTTGTCCAGGTTTGCTAGCAACATTTTTAG |
| rat | GTAACCTAGACCATCATCCCTGGGTTAGTGCTGGCCAACGGT--CCAGCCACATTTTTAG |
| mouse | GTAACTTAGACCGCCATCCCTGGTTTCGTGCCGGCACACGGT--CCAGCCACATTTTTAG |
| dog | GTGACCCAAAAGATGCCCCTTGGTGC----CCTGCAAACTGC--CCAGCAACATTTTTAG |
| human | GTGACCTGGAAGATGTTCCTTGGTCCAGTTTCTGCAAACTGC--CCCGCAGCATTTTTAG |
| opossum | CAAA-CTTTCACTTGTAGATGCAACTACT----CCCAGCCAAATTGAC----TAGGGGGA |
| rat | AAAAATGTTCCCTTTTAGATTTGACTTGTCTTACTTAGCCAGATTGACAGGGTGGGGG-- |
| mouse | AAAAACGTTCCCTTTTAAATTTGACTTGTCTTACTTAGCCAGATTGACAGGGCGGGGG-- |
| dog | AAAA--GTCCCCTTTTAAATTCAATTTGT----CTTCGCCAAATCGAC----CAGGGC-- |
| human | AAAA-TGTTCCCTTTTAGATTCGATTTAT----CTTAGCCAAATTGAC----CAGGG--- |
| opossum | AAAATAAGTCCCTACAAATAGCAATCACTTTGGAATGAGGAGAGTAATTATCTTAAATTG |
| rat | CGGCTAGGTCCCTATGAATAGTGAGCACGCGTGAGTGAGGA--CTCATTATCTTAAATTG |
| mouse | TGGATAAGTCCCTATGAATAGTGAGCACACGTGAATGAGGAGACTCATTATCTTAAATTG |
| dog | AAAATAAGTCCCTACAAATAGCGATCACCAGCGAATAAGGAGAGTCATTATCTTAAATTG |
| human | AAAATAGGTGCCTACAAATAGCGATCACTGGCAAACAAGGAGAGTTATTATCTTAAATTA |
| opossum | AGGCTGGTTTCTGAGGGGAAAA--------- |
| rat | AGGCTGGTTTCTAAGAAAAAGAAAAGGGGAA |
| mouse | AGACTGGTTTCTAAGAAAAAGAGAAAGGGGA |
| dog | AGGCTGGTTTCT------------------- |
| human | AGGCTGGTTTCTAACAACAAAAAAAAACCCA |

TABLE 12
ECRB 320 bp, 77% ID

| | |
|---|---|
| opossum | CAGAGGATATCACACTGAAAGTTCATGCATTTTAAATTGATGTGAAACTTCATATTTTCT |
| rat | GGAGAGATCTCCCACCGGAAGTAGGCTTGCTCCAAATTGATGCCTGACTGGATGCCTTCT |
| mouse | AGAGAGATTTCCCGCTGGAAGTAGGTATGCTCCAAATTGATGCCTGACTGGATGCTTTCT |
| dog | GAGAGGATTTCACACTGGAGGTACATGCATTCCAAATTGATACCTAACTGTATATTTTTT |
| human | GAGAGGATTTTGCACTGGAAGTACATGCATTCAAAATGGATACCTAAGTGTATATTTTCT |
| opossum | AGTGCAAACTATATGTTGAGTCTAATTTTAACCTCAGAGAGCTTTATTATTCTTTTCCTT |
| rat | GCTGTAAACTATATGTCGACTCTAACTTTAGCCCGGGGGAGCATTCTTACTTG------T |
| mouse | GGTGTAAACTATATGTCGACTCTAACTTTAGCCCAAGCGAGCGCTCTTATTTG------T |
| dog | GGTGTAAACTATATGTCGGCTCTAACTTTAGCCTGGGGGAGCTTTGTTATTTGTTTCCCT |
| human | GGTGTAAACTATATGTCGACTCTAACTTTAGCCCGGGGGAGCTTTATTATTTGTCTCCCT |
| opossum | TTAGTGAAAGCTATAATATAAGTAGGAAAAAAAACTT-GACTTGTTCTCTCCATTTCCTG |
| rat | TTCATGAAAGCTATAATACAGGACG---AGACAACTC-GCCTCATACATTCCGATTTCTA |
| mouse | TTCATGAAAGCTATAATACAGGACG---AGACAACTC-ACCTCATACATTCCGATTTCTA |
| dog | TTCATGAAAGCTATAATAGAGGAAG---TGAAAACCTTGCCTTGTACATTCCAATTCCTA |
| human | TTCATGAAAGCTATAATAGAGGAAG---AGAAAACCCTGCCTCGCACATTCCGATTCCTA |
| opossum | AATATACAATTTTACAATTTTCTGAGATATTATTTAGACTTATTTTTACTTTTCAGCCTT |
| rat | AATACACAA-TTTACAATTTTCTGGGATATTATTTAAGTTTATTTTTGTGATGGAGTCTC |
| mouse | AATACACAA-TTTACAATTTTCTGGGATATTATTTAAGTTTATTTTGGTGATGGAGTCTC |
| dog | AATACACAA-TTTACAATTTTCGGGGATATTATTTAAGTTTATTTTGATTCTGGAGTCTC |
| human | AATACATAA-TTTATAATTTTCTGGGATATTATTTAAGTTTATTTTAGTTCTGGATACAC |
| opossum | TCCATCTCCACATTTTTTTCCCTTTAAGTTCCGGGTGGGATTTGCAAAAACATGAAATG- |
| rat | GCCATGCCCGTTGAGCGCT-TATTTAAGTTCTGGGTGGCCACTGGAAAAGCCTGGGATGC |
| mouse | GCCATGCCCGTTGAGCGTT-TATTTAAGTTCTGGGTGGTCACTGGAAAAGCCTGGGTTGC |
| dog | ACCATCCCCATGGGTAGCT-TATTTAGGTTCCGGGTGGACTCTAGAAAGGCCTGGAATGT |
| human | ACCATCCCCGTGGGGTGCT-TATTTAAGTATCGGGTGGGCTCTGGAAAGGCCTGGAATGC |

TABLE 13
ECRC 246 bp, 71% ID

| | |
|---|---|
| chicken | ------------------------------------------------------------ |
| opossum | ACTTTTCTTCCCTGAAACTTAAGAGGTGGAGACACC-------------CCTCTTACACA |
| rat | ----------------------------------------------GGA-TTCCCACCGG |
| mouse | ----------------------------------------------AAA-TTCCCACCAG |
| dog | ACAGCCTTTTGCTGGGATTTGACAGGGAAAAACACAATGGTTGATGGTT-CCCCTACAAG |
| human | ACAGTCCTTTGCTGAGATTTGACAAGGGAAA-CACAGTCATAGATGAAA-CCCCCTACAG |
| chicken | ------------------------------------------------------------ |
| opossum | C--TGCT-------TCTG-CTGAAGCTCAGGAAGGGAGATAAGACTGATCTTTG-AACTC |
| rat | CATGGCTAAACAGCCTTC-CCAAAATC----------CGTGGGCATGACGTCTG------ |
| mouse | CGTGGCTAAACAGCCTTC-CCAAAGTC----------CGTGGGCGTGGCGTCTGAGACAG |
| dog | C--TGCTGCAAAGCCCTGACAGAAA------------AACTAGATTGGCCACTG-AACAG |
| human | C--TGCTGCAAAGCCCTT-CAGAAA------------CATGAGATTGGCCTCTG-AACCA |
| chicken | ---------------GGACCCTGCTGGCGACTGACA---------GCAGCAGAGCCACGG |
| opossum | TAGAAAA-CAATCTGGGACT--------GACTGACA---------TCTGTTGTCTAGTAA |
| rat | ---GGCA-TGGTGTGGGGCT--------GCCGGGCACAGCCTCGTGCGATCCGTGCGTGG |
| mouse | AAGGGTA-TGATGTGGGGCT--------GCCAGGCATAGCCAGGTGTGATCTGTGCGTGG |
| dog | GAGGAAA-CAACGTGGGGCT--------GCCCAACA---------GCGGCCACCCCACGA |
| human | GAGGAAAGTAACGTGGGGCT--------GTACAACA---------GCAGCCAGCCTGTGA |
| chicken | GACTGCTGGAGAAAACTTTAATTTATTTGTACAGCATGCATATGGCAGG--TCCC-CA-- |
| opossum | GATTGATGGGTAAAATTTTAATTTCTTTGGGCGGTTTGTATTTGGAGGGTCTCCC-TGAC |
| rat | GAGTGACAGCCAGGATTTTAATTTGCCCATGCACCGTGCTTAGAGCTGACTTTCC-TG-- |
| mouse | GATTGATAGCCAGGATTTTAATTCTCCCAGGCACCGTGCTTAGAGCTGGATTTCC-TG-- |
| dog | GATTAATGGGTAAAACTTTAATTTATCCAGGCACCTTGCTTTCAGAGAGCCTCCCCTG-- |
| human | GACTGATGGATAAAGCTTTAATTTATCGGTGCACCTTGTTTGGAGAAAGCTTCCC-TG- |
| chicken | ------CC-------------------------------TCCTGTTTCGGTAATGATAAA |
| opossum | TCCGGCCCCTATCCCTTACCCCCACCAGACCCCAACC--TCTGATTTTGCTAATGATAAA |
| rat | ------GCCTCTC-------------------CAACCCCTCCTGGTTTGCTAATG----- |
| mouse | ------GCCTCTCAGC--ACCTCAC----CTCCAACCCTTCCTGGTTTGCTAATGGCAAA |
| dog | ------CCCTCTTGGCTGCCCCTAA----CCCAAACCCCTCCTGTTTTGCTAATGATAAA |
| human | ------CCCTCTTGGCTGCCCCCAC----CTCCAACCCCTCCTGTTTTGCTAATGATAAA |
| chicken | TGACAAGATGTCGACAGTTATTAATTAAAGCACCCGGTGTAGCAA--CGCCAACCAGAAG |
| opossum | TGACAAGATGTGGACAGTTATTAATTAAACTGTCCATAGCGATAAGGTCTGAGAATGTTA |
| rat | --ACAAGCAATGGACATTTATTAATTAAACCGCCCACAGAGGCCAGTGGTAAAACCGAAG |
| mouse | TGACAAGCAATGGACATTTATTAATTAAACTGCCCACAGAGGCCAGTGGTAAAACCGAGG |
| dog | TGACAAGTAGTGGACATG-ATTAATTAAACCGCCCCCAGTAGCCAGTGGTAAAACCCATG |
| human | TGACAAGCAGTGGACATTTATTAATTAAACCACCCGCTGTAGCCATTGGTAAAACCCAAG |
| chicken | G------GCTGGGGAAGCAAAATGTGCAG-----CATTCC-----TTCCTCCC------- |
| opossum | TCCA---GCAGAAGAAA-----TGTGTGGCATTCCTTCCCACCCACTCCCTCCTCCCTTC |
| rat | GCCACATGCAGCGGAGG-----------------CTTTCAAAGCATTCCTACTGATCAT- |
| mouse | GCCGCATGCAGTGGAGG-----------------CTTTCAAAGCACTCCTACTGTTTGT- |
| dog | GCCACAAGCAGAGGAGA-----------------CATTCAAAGCATTCCCACTGCCCCT- |
| human | GCCACATGCAGGGGAAG-----------------CATTCAAAGCATTCCTGCTGCCCCT- |
| chicken | ----------------ACCCTGAGAATGG-------GGCCC------------------- |
| opossum | ACTTCCTCCCCATACCATCCCATGAGCAGGA-----GGCCC------------------- |
| rat | ----------------GCCTGGGGCCCAGCCCAGAGAGCCA------------------- |
| mouse | ----------------GCCTGG--------------AGCCA------------------- |
| dog | ----------------GCCCAGGGCACAGGCCCCAGGCTCCTTCCTGCTCTGGCTAGAAC |
| human | ----------------GCCCAGGGCACAGGCCCCAGGGTTA-TCCTTTGCTGGAGAGAGC |
| chicken | ---CTCCACCGTTCTCGGGCACAGCAGAGATTAGGGGATGGCAGAGGTATTCCTGCACGA |
| opossum | ---CTTTCAGATCCCTGGCC-CAGCAGAGGCCGGACTCACCCAGCAATATCCTCCAACAC |
| rat | ---CCAGGGCAGCCTTGGGT-TAGTGCAA---GGGTCTTCCCAGGAC------------- |
| mouse | ---CCAGGGTAGCCTTGGCC-TAGCACAA---GGGTCTTCCCAGGAC------------- |
| dog | AGGCCCCAGTGGCCACGGCC-TCGCACAA--AGGGCCTTCCTAGGAA------------- |
| human | AGGCCACGGCGGCCACAGCC-TCACATGA--TGGGCCTTCCTAGGAT------------- |
| chicken | GCCAGCTCCCTAGTAACTGCCTGAGCACGGGGCCTGAAACATTGAAATCCTCTCTCA-TG |
| opossum | CTTGGCTACCTAATAACGGTCTGATTCAAG-GGCTGAGAAATGGAAATCTCTCCTGGGGC |
| rat | -----CTGTCTACTAC---TCTGAAT------TCTGACTAATGAAAACCATCTCTGC-AG |
| mouse | -------------------TCTGAAT------TCTGACAAATGAAAACCACCTCTCC-AG |
| dog | -----CTG-----------TCTGGGCTCAG-CTCTGTCAAATGGAAATCACCAATACGGG |
| human | -----CTG-----------TCTGGGTTCTG--GCTGATAAATGGAAATCACCTCCATGGG |
| chicken | CCATGTGCAGT-----AATTAAACTTC-GGCATTCCTTCTGC--AGGAAAT--------- |
| opossum | CTG--CACAGT-----AATTAACCTCAGAGCATTCTTGAGGC-AGGGAAAT--------- |
| rat | CCA--TGGGAT-----AATTAAACTGCCTGCATTTCTTTAAAAAAAAAAAAAAAAGTCAA |
| mouse | CCA--TGGGATGGGATAATTAAACTCCCTG-------------AAAAAAAAAAAAATCAA |
| dog | CCA--CACAGT-----AATTAAACTCCTGGCATTCTTTTGAC--AAAAAAT--------- |
| human | CCA--CACAGT-----AATTAAACTCCTGGCATTCTTTTGACAAAAAAAAA--------- |
| chicken | -----GTTTCTCCTGTGTTGTTCCCACAGAT--TGTGCTACAACAACAATTTTAGCCCCG |
| opossum | -----GTTTCTCTTGGGACATTCCCACAGGCCATG---TGTAATACCAGGCTTAGCCCCA |
| rat | AGATCGTTCCTCTAGGGACATTCCCACAAGC--TG---CAGAAGGACT---TGGGAGTTT |
| mouse | AGATCGTTCCTCTAGGGACATTCCCACGAGC--TG---CAGAAGGACTGGCTGGGATCTT |
| dog | -----GTTCCTCTTGGGACATTCCCACAGGC--TG---TTGAAGGACTGTCGGGGCCCTA |
| human | -----GTTCCTCATGGGACATTCCCACAAGC--TG---CTGAAG----GTCTGGGCCCTG |
| chicken | TGAACTATTAACCAATTATAAAGGGATTTTCTCAGTCCGCTTTAAC---TGAGAATCA-- |
| opossum | TGAG-GCTAGGCTGATTATGAAGGGATTTTCACAGTTGGCTCTAAC---TGTGGATCATG |
| rat | TGAGCTCTCAGCT-AGGACTGAGGGATTTT-GAAGTTGGCCTTGAC---AGGAGATTT-- |
| mouse | TGAGCTCTCAGCT-AGGACTGAGGGATTTT-GAAGTTGGCCTTGACTGGAGGAGATTG-- |
| dog | TAAGCTCTCAGCTGATTACCAAGGGATTTTTGAAGTTAGCCTTGAC---TGTGGATCA-- |
| human | CAAGCTCCCAGCTGATTACCAAGGAGTTTTCGAAGTTGGCCTTGAC---TGAGGATCA- |
| chicken | -------------------------------------------------------- |
| opossum | AAGATGGAGGAGA------------------------------------------- |
| rat | GAGGGGGAGGAGAGTC---TGGGTG--GGTG--GGTAGGAAATGCCTTGTAATTAA |
| mouse | GAGGGGGAGGAGAGTT---GGGATG--GGTG--GGTAGGAAATGCCTTGTAATTAA |
| dog | AAAGGGGATGGGAATTCACGGGGTGCTGGTGGAGATGGGAAGTGCCTTGTAACAGA |
| human | AAGGAGGATGGGAGTTCAGGGAATGAGGGTGGGGGTGGGAAATGCCTTAGAATTAA |

TABLE 14
ECRD 348 bp, 78% ID

| | |
|---|---|
| opossum | ------------------------------------------------------------ |
| rat | TTCCTTCTGTCTCT------GTCTGACTCTCAGACTATTAATACAAGCCT---------- |
| mouse | TTCCTTCTGTCTCTCTGTCTGCTTGACTCTCAGCCTATTAATACAAGCCCTGTGATTAAT |
| dog | TTCCCTCTGCCCCTCTGTCTGACTC---CCAAGACTATTAATACAAACCC---------- |
| human | TTCCTTCTATCCCTCTGTCTGTCTTACTCTCAGACTATTAATACAAGCCC---------- |
| opossum | ------------------------------------------------------------ |
| rat | --------CATGTCTGGCTGCGCCCCAAGCACACGTGCTCC-----CTGCAATGGAGTGC |
| mouse | ACAAGCCCCGTGTCTGGCTGTACCCCAAGCACACGTGCTCC-----CTGCCATAGAGTAC |
| dog | --------C-AGTCTGGCTGTGCCACCAGAACATGTGCTCC-----CCACAGCAGAATGC |
| human | --------TGAGTCTGGCTGTACCCCCAGAACATGTGCCCCGCCCCCTACAACAAAATGC |
| opossum | ------------------------------------CCGATTAGCTTCAGGCTCACAAAT |
| rat | CACCCCTCCCAGCTCAGTCCTTTGTTTGTTC--TTTCTGATGGGCGCCAGCCTCATAGAC |
| mouse | CACCCCTCCCAGATCAGCCTGTTGTTTGTTC--TTTCTGATGGGCGCCAGCCTTGTAGAC |
| dog | CACCCCTCCCAGCTGAGCCTGTTGTCTGTTCCGTTTCTGATCGGCGCCAGGCTCATAGAC |
| human | TGCCCCTCCCAGCTAGGTCTGTTGTTTGTTCCTTTTCTGATTGGCGCCAGGCTTATAGAC |
| opossum | CCCATATGGTTAGTATATGACATTCCATAAATAGCCCAAAACACGCCCTACAATTTAGCC |
| rat | CCCATAGAGGTAGAATATAACTTTCCATAAATAAACTCTAACCTGACCTACAATTTAGAC |
| mouse | CCCATGGAGGTAGAATATAACTTTCCATAAATAAACTCTAACCTGACCTACAATTTGGAC |
| dog | CCCATGTAGGTAGAATATAACTTTCCATAAATAACCCCTAGCCTGACCTACAATTTAGGC |
| human | CCCATGTAGGTAGAATATAACTTTCCATAAATAACCTCTAACCCGACCTACAATTTAGCC |
| opossum | TGTGG----GTTTTCCCCCTCATGCTAACAGGA-------------CAGGCCCTCCTGGG |
| rat | CTTGGGCTCCCCCCCCCCTTTGTGGTAATGGGATCATAGCCCAAGCTAGTCTCTCCTGCA |
| mouse | CTTGGNNNNNNTTCCCTCTTTGTGGTAATGGGATCACAGCCCAACCTAGTCTCTCCTGCA |
| dog | TTTGGGTTTTTTT--CCCCTCGTGGTAATGGGATTATAGCCTGGGCTGATCCCTCCTGGG |
| human | TTCAGGTTTTTTT-CCCCCTCGTGGTAATGGGATTGCAGCCTGGGCTGATCCATCCTGTA |
| opossum | TCCCCAGCCTCTATGAAAGTATCCCCTTTGCTGGCCTCTTTGTAGGGCATTGTCTACCTT |
| rat | TCTGCCCATCCCA-GAAAGCAAGCCCTAGGTGTGCTT-GGGTTTAGGCATCACTTGGACT |
| mouse | TCTGTCTGTCCCA-GAAAGCAAGTCCTAGGTGTGTTT-GGGTTTGGGCATCACTTGGACT |
| dog | TCTTGGGGTCCCA-GAAAGCAGACCCTAGGTGTATC---GGGCTGGGCATTGCTTGGAAT |
| human | TCTTCAGGTCCCA-GAAAGCAGACCCTAGGT-----------TTGGACATTGCTTGGAAT |
| opossum | CTTTGAT--C-CTCAC |
| rat | CTCTGAT--CCCACAT |
| mouse | CTCTGATGCCCCTCAT |
| dog | TGCCGGT-TCCCCCAT |
| human | TCCTGGT-ACCCCCAT |

TABLE 15
ECRE 600 bp, 77% ID (human to mouse)

| | |
|---|---|
| opossum | ------------------------------------------------------------ |
| rat | TGTGA----TGGAAGGAGCCCACAGGAGGGGTGGGCTAAGGGGAAGATGGGGC-TGTCAG |
| mouse | TGAGA----TGGAAGGAGCCCACAGGAGGGGTGGGCTGAAGGGAAGATGGGGC-TGTCAG |
| dog | TGGGGGCTCTAGAAGGA-TCCACAGGA------GGCTGGGAGGAAGCCCTGGCCTGTCAG |
| human | TGGGGGTTCTGGAAGGAATCCGTGGGA------GGCTGGGAGGAAGATCTGGCTTGTCAG |
| opossum | ------------------------------------------------------------ |
| rat | C-TCCTTAGGAAGCCCTG----GCAGTA------GGCAA--GCTGTAACAAGATCCCTGT |
| mouse | C-TCCTTAGGAAACCCTT----GGGGCCTGACCTGGCAA--ACTGTAACCAGATCCCTGT |
| dog | CTTCCCCAGGCAAACCTTCCCGGGGGCCTGGC--CCCAGCGGCTGTAACCGGACTCCTGC |
| human | CTTCCCTAGGAAAACCTTCCCCTGGGC-TGGC--CGCAG--GCTGTAACCGGATTCCTGC |
| opossum | --------------------------------------------------CCC----C-T |
| rat | TGGGCCATCCTTCTCACATCTGGCTCAGGGTC--CCCATCCCAGGGAGGC-CC----C-G |
| mouse | TGGGCTGTCCTCCTCACATCTGGCTCAGGGTC--CCCATTGCAGGGAGGC-CC----CCT |
| dog | T---CCGGCCTC--CGCAGCTGGCCCTGGGGAGGCCCAGCGCGGGGAGGC-CCAGC-C-G |
| human | T---CCA-CCTC--TGCATCTGGCCCAGGGAC--CTCATGGCAGGGAGGC-CCAGCGC-C |
| opossum | TGGGGCTTGGCAGCAGGGCTATGAAATGGGATGTGAG---CAGTGGGGTATA-TAGA-GA |
| rat | TGGCCCTTTGCCCTGGG---AAGGGGTGGGCACTGGGTCACGGTGGGGGGGGGTGGGNGA |
| mouse | TGGCCCTTTGCCCTGGG---AAGGGGTGGGCACCGGGTCACGGTGGGGTGGG-TGGG-GA |
| dog | CGGTCCCTGGCCCCTGG---AAGGGGCGGGCC-----------------------GA-GG |
| human | TGGCCCTTTGCCCCTGG---ACGGGGTGGGCCCTGGGTCATGGTGGGGTGGG-TGGG-GA |
| opossum | AGTGGTAGGTGGTTAGGG----------AGCAGTGCTCTTGGTGGTGAGAACATGCTGG- |
| rat | GGTCANAAAGGGCATGGGGCGGGG-CACAGCAGGGTGCTATGTGCTGGGAACACA--GG- |
| mouse | GGTCAGAAAGGGCATGAGGCGGGGGCACAGCGGGGTGCTATGTGCTGGGAACATA--GG- |
| dog | GGTCGGGGGG--CAGCGGGAGGCGCGGCGG-GGGGTGCGGTGCGCT-------------- |
| human | GGTCAGGAGGGCCATGGGGAGGGGGCGCGGTGGGGTGCTTTGCCCTGAGAACACA--GG- |
| opossum | -CCCC--GCAGTCCC------CCCCGAC-------------------------------- |
| rat | GCCTG--GCA-----------CCCCAGT-------------------------------- |
| mouse | -CCTG--GCA-----------CCCCGGT-------------------------------- |
| dog | -CCTC-----------------CTCGGGCCGNNNNNNNNNNNNNNNNNNNNNNNNNNNNN |
| human | -CCTCTGGCA-----------CCCCGGA-------------------------------- |
| chicken | ---------------ACTCTCT-GCAGCTGCTGCTGTCACTCAGCGGCCCAGCGGGG--- |
| opossum | ---------------ACCCTCA-GCAGCTGCCTGTGTCTGTCAGCCGCCCCTCGGGGG-- |
| rat | ---------------GCCCCA--GCAGCTGCTGGCGTCTGTCAACCCCCC-TGGGGGCAC |
| mouse | ---------------GCCCCA--GCAGCTGCTGGCGTCTGTCAGCCCCCCCTAGGGGCAC |
| dog | NNNNNNNNNNNNNNGGCGCCCGCGCAGCTGCCCGCGTCTGTCA----------------- |
| human | ---------------GCCCCCG-GCAGCTGCTGGCGTCTGTCAGCCACCTTGCGGGGCGC |
| chicken | -----GCTTGTT----------------------------------TGCCCTCTGCATCT |
| opossum | -----GGTTGTT----------------------------------TGCCCTCTACATCT |
| rat | CACAAGGCCA------------------------------------CCACCTCTACATCT |
| mouse | CACAAGGCCG------------------------------------CCACCTCTACATCT |
| dog | -----------------NNNNNNNNNNNNNNNNNNNNNNNNNNNNNTGGCCTCTACATCT |
| human | -----GGCCGGGGGCTGC----------------------------TGGCCCCTACATCT |
| chicken | TCCTGACA--CTTCATTCCTGAAG-GAAGCAAAAACAACA---GCCCTCCCCTC-TTGCC |
| opossum | TCCTGACAGTTGGCTCTTCTGAAACCAGAAAAAAACAACA---A-TTCTTCCTTGTCACT |
| rat | TCCTGACAGGCCCCTCTTCTGAGGCCAGGAAAAAACAACA---G---CCCCCTC-CTACA |
| mouse | TCCTGACAGGTCCCTCTTCTGAGGCCAGGAAAAAACAACA---G---CCCCCTC-CCACA |
| dog | TCCTGACAGGCCCCTCTTCGGAGGCCAGGAAAAAACAACA---G-TCCCTCCCC-TCGCG |
| human | TCCTGACAGGCCCCTCTTCTGAGGCCAGGAAAAAACAACAACAG-TTCCTCCCC-TCACG |
| chicken | CAAGCCCATTTGTTGGACG-AGGCAGC-------CACCTTTAACCTCTGCAGAGTCAACA |
| opossum | GAAACCCATTTGTTAGATG-CTGCTGGGGACTGGCACCTTTAACCTTCTTAAAGTCAACA |
| rat | GCAACCCATTTGTCAGCTGAAGGCTGT--CCCTGCACCTTTAACCTCCTCAAAGTCAGCA |
| mouse | GCAACCCATTTGTCAGCTGAAGGCCGG--CCCTGCACCTTTAACCTTCTCAAAGTCAGCA |
| dog | GCAGCCCATTTGTTAGATGAAGGCCGGGCACCGGCACCTTTAACCTCCTCAAAGTCAGCA |
| human | GCAACCCATTTGTTAGATGAAGGCCGGGCACCAGCACCTTTAACCTCCTCAAAGTCAGCG |
| chicken | TCTCTT-CTGCCAGACCCCAGGCTGGGCGG-----GAAACATAGGATGGATGG-GTGCAA |
| opossum | TTTCCCTTGTCA---CTAT--GCAGGTCTGGATAAGAGGCC-AAGATGGATGGAAAACAA |
| rat | GTTCCC-TGTCAAGGCCCT--GTGGGGCCAG----GAGACA-AAGATGGATGG-AGGTGA |
| mouse | GTTCCC-TGTCAAGGCCCT--GTGGGGCCAA----GAGACA-AAGATGGATGG-AGGTGG |
| dog | TTTCCC-CCTCCCGGCCCC--GC-GGGCCC-----CAGACA-GAGATGGATGG-AGGCGG |
| human | TTTCCC-TGTCAAGGCCCC--ACAGGGCCA-----GAGACA-GAGATGGATGG-AAGGAG |
| chicken | TAGGTTTCAAA-------------------------------- |
| opossum | TTGTTTGCAAAA-GAAACTTCTTTAGTGAA--CTCAA------ |
| rat | CTGTTTGCAGGG-GAAAGCTCTGGGCAGCA--GCCAAGGGGAG |
| mouse | CTGTTTGTAGAG-GAAAGCTCTTGGCTGCA--GCCAAGGGGAG |
| dog | CTGTTGGTGGAAGAAAGGCCCGTGGCTGCGCGGCCAAGGGGAG |
| human | CTGTGTGTCGAA-AAAGCCCTGTGGCCTCATG----AGGAGAG |

TABLE 16
Sost promoter 341 bp, 84% ID

| | |
|---|---|
| opossum | AGTCCTT-----GGAGCACTTTCTCCTTTTTTAAATCCCC-CTTTGCCGCTTCACCAGCC |
| rat | AGTCCCCAGCC-CAGATACGGTCGCC-TTTTAAAAGCCCC-CTCTACCTCAT-GCCAGCC |
| mouse | AGTGTCCAGCC-TAGATACGGTTGCC-TTTTTAAAGCCCC-CTCTGCCTCAT-GCCAGCC |
| dog | GGTCCCCAGCCTGGGACCTGTTTGCC-TTTTTAAAGCCCC-CTCCGCCTGAT-GCCAACC |
| human | GGTCTCCAGCC-GAGACACGGTCGCC-TTTTTAAAGCCCCTCTCTGCTTCAT-GCCAGCC |
| opossum | AATGGGGTTACAGAC-AGAGAAAGGCCAGCCCCACATTTC-------------ACTTGGG |
| rat | AATAAGG--ACAGGCTGGAACAGAGCTGGTTCCATGTTTCCTCACCCTCCTCCCCATGGG |
| mouse | AATAAGG--ACGGGCTGGAGCAGGGCTGGTCCCATGTTTCCTCACCCTCCTCCTCACGGG |
| dog | AATGAGG--ACAGGCTGGGGCAGGGCTGGTCCCATGTTTC--------TCTCAGCCCAGG |
| human | AATGAGG--ACAGGCTGGGGCAGGGCTGGTCCCATGTTTC--------CCTCAGCCCCGG |
| opossum | ----GGGAGGGGTGGTAGGAAATCGAGAGGGAAGGCAAAGGGGTGTGTTCAGAGCCAACC |
| rat | GGTGGGGAGAAG---------------------------GGGGTGTGCCCAGTGTCAGCC |
| mouse | GGTGGGGTGGGG----------------GGGGTGGAGAAGGGGTGTGCTTAGCGTCAGCC |
| dog | ----GGGAGGGA-------------------------ACGGGGTGTGCTCATGGCAAGCC |
| human | ---AGGGAGGGA-------------------------AAGGGGTGTGCTCAGAGCAAACC |
| opossum | CACTTC-ACCACTTTTCTTCCCAGGCTGTCTAAACTTCCAAATAGCTCCAGTCTCTTCCC |
| rat | CTCCCC-AAGACTTCTCA-----AGCTCAGCAAACTTCCAAATTGCTGCTGGCA-CTCCC |
| mouse | CT-CCC-AAGACTTCTCA-----AGCTCAGTAAACTTCCAAATTGCTGCTGGCACCTCCC |
| dog | CTCCCCGAAGACTTCTCA-----GGCACAGCAAACTCCCAAATTGCTGCTGGTG-CTCCC |
| human | CTCCCCAAAGACTTCTCCTCT--AGCTCAGCAAACTTCCAAATTGCTGCTGGCA-CTCCC |
| opossum | AGGTAGTTTGGAATGTCTAGGTTCATGCCTGGCAGATCCATAAG-GAAGAG-----GGGT |
| rat | AGGTGACCCGAGGAATG-GGGTGGGGGTGGGGGGGGTTGGTGAG-GCAGGGACACAGCCA |
| mouse | AGGTGACCCGAGGAGTGTGTGTTGGGGGGGGGGGCGTTGGTGAG-GCAGGGACACAGCCA |
| dog | AGGTGACCTAGAGGGAG-------------GGGGAGT--GTGTGTGTGAGG--CAAGCCT |
| human | AGGTGACCCAGAGAGAG-------------GGGGCGT--GTGAG-GCAAGGCCCAAGCCT |
| opossum | CCTTT-TCAGCGTC-------------------CAGCTCT--TTTTCGGTTATTCAAAAG |
| rat | GCTCTCCCAGCACCTCCCACAGGCAGA------CGGCTGTGGTTTTCAGATA-TCAAAAT |
| mouse | GCTCTCCCAGTACCTCCCACGGGCAGGGCAGTCAGGCTGTGGTTTTCAGATA-TCAAAAT |
| dog | GCCCT-CCAGCACCTCCCACGTGCAGG------CGGCTGTGGTTTTTAGATA-TCAAAAT |
| human | GCTCT-CCGGCACCTCCCACGTGCTGG------CGGCTGTGGTTTTCAGATA-TCAAAAT |
| opossum | CTGCTTTCCTCCTCAGACGGATGACGTCTCTGGACCCCTTTATCTGTCTC-AAAACAAGC |
| rat | GAGCT-CGGCTTGTAATTGTCTGGC-TCCCTGGGCCCCTCAGGCATTCTC-AAAACAAAC |
| mouse | GAGCT-TGGCTTTTAATTGTCTGGC-TCCCTGGGCCCCTCAGGCATTCTC-AAAACAAAC |
| dog | GAGCTCCAGCTCGTAATC-TTTACC-TCCTTGGGCCCCTTGGGCTTTCTCAAAAACAAAC |
| human | GAGCTCCGGCTTTTAATTGTCTGTC-TCCCTGGG-CCCTCGGGCATTCTC-AAAACAAAC |
| opossum | TGGATCCCAGTTTAACAAGCAAACTTCC------CAAGTCTCCTTCAGGAAG-------- |
| rat | TGCGGGCTAGCTTAACAAAGGAAACAAG------TGGGGCTTCTACAGAAGGCAGAATCC |
| mouse | TGTGGGCCAGCTTAACAAAGGAAACAAG------TGGGGCTTCTACAGAAGGCGGAATCC |
| dog | TTCGGACCAGCTTCACGAAGAAAACGAG-AGCTCTGAG---CCCACAG--GGCTGAGCCC |
| human | TGTGGACCCGCTCAACAAAGAAAATGTGCAGTTCTGAGTGCTTCGCGG--GGCTGAGCCC |
| opossum | ------------------------------------------------------------ |
| rat | GAGGGAGGGTTCACACCTGAGGTGCAAAAAAAGGAGGG---GCTGGCTGGCGGAAGC-TT |
| mouse | GAGGGAGGGTTCACACCTGAGGTGCAAAAGGAGGAGGG---GCTGGC----GGAAGC-TG |
| dog | GGAGAAAGGTTCACACCTGAGGTGCAGGAAAAGAAAGGAGAGCAAGC----GGAAGCTTT |
| human | GGAGAAGGATTCACACCTGAGGTGCAAGAAGAGAAAGGAAGGCCAGA----GGAATCTTT |
| opossum | ------------------------------------------------------------ |
| rat | CACTG-AGACCGAGTGTTGGAGGACGCCCCAGCCCTG----------GAGGAAGGCTGGG |
| mouse | CACTG-AGACAGAAGGTTGGAGGACTCCCCAGCCCTG----------GAGGAAGACTGGG |
| dog | CACTGCAGATGAA-----GGAGGAGA-CCCACCCCGCTCCTGGCCCCGAAGGAGGCTGGG |
| human | CATTGAAGACAGATTGTTTGAGGACA-AGCAGCTC--------------------CTGTG |
| opossum | ------------------------------------------------------------ |
| rat | AAGAAGGCTGTGGTCCACCTTGCCT----TGGTATGCTAACTGGGCAATGAATCAGGGTC |
| mouse | AAGGAGGCTGTGGTCCGCCTAGCCCGCTGTGGTATGCTAACTGGGTAATGAATCAGTGTC |
| dog | AAGAGGGCTGCAGCCCAGGTCCCCT----TGGTATTTTCACTGGGCAATGAATCAGCACG |
| human | AAGAG----GCTGCCCAGATCCTC-----TGGTATTTTAACTGGGCAATGAATCAGCATG |
| opossum | ---------------------------CTTTGCCGCCTGCCTCGACTTGCAAAGTCAACC |
| rat | -ATCCACCTGCAGAGCTTCAACAAGG-CTCCGCCACCTGCCTTGACTTGTAAG--TGGCC |
| mouse | -GTCCACCTGCAGAGCTTCTGCAAGG-CTCTGCCACCTGCCTCGACTTGTAAG--TGGCC |
| dog | CCCCCACCTGCAGAGCCTCTAGGAGGCCTCTGCCACCTGCCTTGACTTGTAAG--CGGCC |
| human | -CCCCACCTGCAGAGCCTCTGCAAGTCCTTTGCCACCTGCCTCAACTTGCAAG--TCGCT |
| opossum | T-GTCCAT-GGTGCCCCTCCGCCTCGGTCCCTCTTTCTACAGATCAATCATTGAAGGTGC |
| rat | T-GTCC------------------------TCCTTTTCATC-TTCTGTCACCC---ACAC |
| mouse | G-GTCCATCTGTGGCGATCTGCCTGGGTCCTCCTTTTCATC-TTCTGTCACCC---ACAC |
| dog | GTGTCCATCTGCAGCCGTCTGTC-----CCTCCTATCCACC-ATCCATCACTC--CACAC |
| human | GTGTCCATCTGCAGCCGACTGT------CCTCCTTTCCATC-ATCCATCATCC--GACAC |
| opossum | TTGAACGCTGCCGCCCTGACAGTTTTACAAGACAGGCAGAAGCCCCCTCAGGAAGGAGTT |
| rat | ATCAGTGCAGCTGCTCTGACAGCTTTACAAGGCAGACCGAAGTCCCCTCAGGAAGGG--G |
| mouse | ATCAATGCAGCTGCTCTGACAGCTTTACAAGGCAGACAGAAGTCCCCTCAGGAAGGG--G |
| dog | ATGAATGCAGCTGCTCTGACAGTTTTACAAGCCAGACAGAAGCCCCTGCAGGAAGGGCCC |
| human | ATGAATGCAGCTGCTCTGACAGTTTTACAAGCCAGACAGAAGCCCCCTCAGGAAGGGCCC |
| opossum | TCTCTCATACCTGTTTCCCGACTTAGTTACCTGCAGGGAAGGTGAAAACAGCTGTTGCTT |
| rat | GCTCTGATACCTGCTTCCTAAGTCAGTTTCCTTCGGGGCAGACAAAAACAGCTGTGGCCA |
| mouse | ACTCTGATACCTGCTTCCTAAGTCAGTTTCCTTCGGGGCAGACAAAACCAGCTGTGGCCA |
| dog | TCTCTGATACCTGTTTCCTAAGTCAGTTTCCTTCGGGGCAGACGAAAACAGCTGTGGCCA |
| human | TCTCTGATACCTGCTTCCTAAGTCAGTTTCCTTCAGGGCAGATGAAAACAGCTGTGGCCA |
| opossum | TTATTGGGGG-------------------------------------------------- |
| rat | TTGTTGGGGGTGATCTATCTCTCCTACCAGAGCTGT--GGTGTTGTTAGAAGCTGGCCCT |
| mouse | TTGTTGGGGGTGATCTATCTCTCCTACCAGAGCTGT--GAAGTTGTTAGAAGCTGGCCCT |
| dog | TTGTTGGAGGTGATCTATCTCTCCCAACAGAGCCCCTGGGTGTTGTGAGAAGCTGGCCCT |
| human | TTGTTGGGGGTGATCTATCTCTCCCAGCAGAGCCGGT-AGTGTTGTGAGAAGCTGGCCCT |
| rat | --------------TTTGTCTGTGA-CCTCCAGTCTCCTTTC-CCCCTGCTCTGGAATGT |
| mouse | --------------TTTGTCTGTGA-CCTCCAGTCTCCTTTT-CCCCTGCTCTGGAATGT |
| dog | CTGTGCCCCCTCCCCCAGTCCCTGACCCTCCAGCCTCCTTTTCCCCTTGCTCTGGAATGT |
| human | CCGCGCCACCTCCCCTTGTCTATGACCCTCCAGCCTCCTTTT-CCCTTGCTCTGGAATGT |
| rat | GGACTGTTAACCCTGTCTGGGCCAGAAAGCTGTCCTGTTGCTCCAGACTTGGGAAGAGGG |
| mouse | GGACTGTTAACCCTGTCTGGGCCAGAAAGCTTTCCTATTGCTCCAGACTTGGG-AGAGGG |
| dog | AGGCCGTTAACCCGATCGTGGCCGGACAATTGCCTTCTCGCCCTGGATTTGGG-AGAAGG |
| human | GGACTGTTAACCCTCTCATGACCAGACGACTGCCTTCTTGCAATGGACTTGGG-AGAGAG |
| rat | CAGCTCTTGGACCC-GGATTCTGAGAGCATGCAGAATGGTT |
| mouse | CAGCTCCTGGGCCCGGGATTCTGTGAGCAC-CATAATGGTT |
| dog | CAATTCCGAGG-CT-CCATCCCTGGAGCTGGCGCGATGGGC |
| human | CAGTTCTGATGCCT-GGGTTCCTTGAGCAGGTAGGATGGAT |

What is claimed is:

1. A method of modulating proliferation of a cell of a bone lineage, the method comprising:
contacting the cell with a composition that modulates the function of a SOST regulatory element, wherein the regulatory element is selected from the group consisting of: ERC1, ERC2, ERC3, ERC4, ERC5, ERC6, ERC7, ERC8, ERC9, ERC10, ERCA, ERCB, ERCC, ERCD, ERCE, and combinations thereof.

2. The method of claim 1, wherein the enhancer comprises a sequence selected from the group consisting of: SEQ ID NOS: 1, 2, 3, 4, 5, 6, 7, 8, 9, 10, 11, 12, 13, 14, and 15.

3. The method of claim 1, wherein the SOST-regulatory element is an enhancer.

4. The method of claim 3, wherein the enhancer is ERC5.

5. The method of claim 4, where the ERC5 comprises the sequence set forth in SEQ ID NO:5.

6. The method of claim 1, wherein the composition is selected from the group consisting of: a small molecule, an antibody, and an aptamer.

7. The method of claim 1, wherein the cell is in a vertebrate.

8. The method of claim 7, wherein the vertebrate is a rodent.

9. The method of claim 7, wherein the vertebrate is a human.

10. The method of claim 7, wherein the vertebrate has been diagnosed with a disease or disorder associated with aberrant bone density.

11. The method of claim 10, wherein the bone density of the mammal is increased following contact with the composition that modulates the SOST regulatory element.

12. The method of claim 10, wherein the disease or disorder is selected from the group consisting of: osteopetrosis, ostepenia, osteosclerosis, craniotubular hypertoses, Van Buchem's disease, and osteoporosis.

13. The method of claim 1, wherein the composition inhibits the function of the SOST regulatory element.

14. A transgenic non-human animal having cells comprising a chromosomally incorporated transgene comprising a recombinant polynucleotide encoding sclerostin (SOST) and a recombinant polynucleotide encoding MEOX1 operably linked to a regulatory region comprising a sequence set forth in any one of SEQ ID NOS: 1-15 and 17-59, wherein the animal exhibits altered bone mineral density, limb deformities, and SOST is expressed embryonically and in the adult bone, liver, brain, lung, heart and kidney tissues.

15. The animal of claim 14, wherein the animal is a mouse.

16. The animal of claim 15, wherein all of the cells in the mouse comprise the chromosomally incorporated transgene.

17. A transgenic non-human animal having cells comprising a chromosomally incorporated transgene comprising a recombinant polynucleotide encoding sclerostin (SOST) and a recombinant polynucleotide encoding MEOX1 operably linked to a regulatory region, wherein the 52 Kb Van Buchem deletion region has been deleted from the regulatory region, wherein the animal exhibits altered bone mineral density, limb deformities, and SOST is expressed embryonically in the heart and kidney tissues.

18. The animal of claim 17, wherein the animal is a mouse.

19. The animal of claim 18, wherein all of the cells in the mouse comprise the chromosomally incorporated transgene.

20. An isolated polynucleotide for modulating SOST expression, said nucleotide having 95% identity to at least one sequence selected from the group consisting of SEQ ID NOS: 1-15 and 17-59.

21. An expression vector comprising the isolated polynucleotide of claim 20 operably linked to a gene selected from the group consisting of: Lac-Z, a fluorescent protein, cre-recombinase, and human SOST.

22. A host cell comprising the expression vector of claim 21.

23. A transgenic non-human animal having cells comprising the expression vector of claim 21.

24. A method to determine the genetic status of an individual, the method comprising: detecting a variation in the sequence of at least one SOST regulatory element wherein the regulatory element is selected from the group consisting of: ERC1, ERC2, ERC3, ERC4, ERC5, ERC6, ERC7, ERC8, ERC9, ERC10, ERCA, ERCB, ERCC, ERCD, ERCE, and combinations thereof.

25. The method of claim 24, wherein the wild type SOST regulatory element comprises a sequence set forth in any one of SEQ ID NOS: 1-15.
